(19) United States
(12) Patent Application Publication
(10) Pub. No.: US 2015/0218172 A1
(43) Pub. Date: Aug. 6, 2015

(54) Pyrrolo[2,3-D]Pyrimidine Tropomyosin-Related Kinase Inhibitors

(71) Applicant: PFIZER LIMITED, Sandwich, Kent (GB)

(72) Inventors: Mark David Andrews, Sandwich (GB); Sharanjeet Kaur Bagal, Great Abington (GB); David Graham Brown, Sandwich (GB); Karl Richard Gibson, Sandwich (GB); Kiyoyuki Omoto, Great Abington (GB); Thomas Ryckmans, Sandwich (GB); Yogesh Sabnis, Sandwich (GB); Sarah Elizabeth Skerratt, Great Abington (GB); Paul Anthony Stupple, Sandwich (GB)

(73) Assignee: Pfizer Limited, Sandwich, Kent, UK (GB)

(21) Appl. No.: 14/428,997

(22) PCT Filed: Sep. 26, 2013
(86) PCT No.: PCT/IB2013/058890

§ 371 Date: Mar. 18, 2015

Related U.S. Application Data
(60) Provisional application No. 61/709,482, filed on Oct. 4, 2012.

(51) Int. Cl. C07D 487/04; A61K 31/519

(52) U.S. Cl. CPC C07D 487/04; A61K 31/519

(57) ABSTRACT

The present invention relates to compounds of Formula (I)

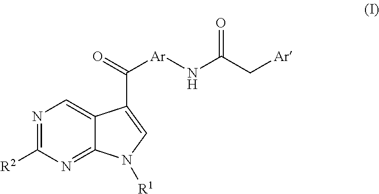

and their pharmaceutically acceptable salts, wherein the substituents are as described herein, and their use in medicine, in particular as Trk antagonists.

The invention described herein relates to certain pyrrolo[2,3-d]pyrimidine compounds and the pharmaceutically acceptable salts of such compounds. The invention also relates to the processes for the preparation of the compounds, compositions containing the compounds, and the uses of such compounds and salts in treating diseases or conditions associated with tropomyosin-related kinase (Trk), activity. More specifically the invention relates to the compounds and their salts useful as inhibitors of Trk.

### BACKGROUND

Tropomyosin-related kinases (Trks) are a family of receptor tyrosine kinases activated by neurotrophins. Trks play important roles in pain sensation as well as tumour cell growth and survival signaling. Thus, inhibitors of Trk receptor kinases might provide targeted treatments for conditions such as pain and cancer. Recent developments in this field have been reviewed by Wang et al in Expert Opin. Ther. Patents (2009) 19(3): 305-319 and an extract is reproduced below.

### “1.1 Trk Receptors

As one of the largest family of proteins encoded by the human genome, protein kinases are the central regulators of signal transduction as well as control of various complex cell processes. Receptor tyrosine kinases (RTKs) are a subfamily of protein kinases (up to 100 members) bound to the cell membrane that specifically act on the tyrosine residues of proteins. One small group within this subfamily is the Trk kinases, with three highly homologous isoforms: TrkA, TrkB, and TrkC. All three isoforms are activated by high affinity growth factors named neurotrophins (NT): i) nerve growth factor (NGF), which activates TrkA; ii) brain-derived neurotrophic factor (BDNF) and NT-4/5, which activate TrkB; and iii) NT-3, which activates TrkC. The binding of neurotrophins to the extracellular domain of Trks causes the Trk kinase to autophosphorylate at several intracellular tyrosine sites and triggers downstream signal transduction pathways. Trks and neurotrophins are well known for their effects on neuronal growth and survival.

### 1.2 Trks and Cancer

Originally isolated from neuronal tissues, Trks were thought to mainly affect the maintenance and survival of neuronal cells. However, in the past 20 years, increasing evidence has suggested that Trks play key roles in malignant transformation, chemotaxis, metastasis, and survival signaling in human tumors. The association between Trks and cancer focused on prostate cancer in earlier years and the topic has been reviewed. For example, it was reported that malignant prostate epithelial cells secrete a series of neurotrophins and at least one Trks. In pancreatic cancer, it was proposed that paracrine and/or autocrine neurotrophin-Trk interactions may influence the invasive behavior of the cancer. TrkB was also reported to be overexpressed in metastatic human pancreatic cancer cells. Recently, there have been a number of new findings in other cancer settings. For example, a translocation leads to expression of a fusion protein derived from the N-terminus of the ETV6 transcription factor and the C-terminal kinase domain of TrkC. The resulting ETV6-TrkC fusions are oncogenic in vitro and appear causative in secretory breast carcinoma and some acute myelogenous leukemias (AML). Constitutively active TrkA fusions occurred in a subset of papillary thyroid cancers and colon carcinomas. In neuroblastoma, TrkB expression was reported to be a strong predictor of aggressive tumor growth and poor prognosis, and TrkB overexpression was also associated with increased resistance to chemotherapy in neuroblastoma tumor cells in vitro. One report showed that a novel splice variant of TrkA called TrkAIII signaled in the absence of neurotrophins through the inositol phosphate-AKT pathway in a subset of neuroblastoma. Also, mutational analysis of the tyrosine kinome revealed that Trk mutations occurred in colorectal and lung cancers. In summary, Trks have been linked to a variety of human cancers, and discovering a Trk inhibitor and testing it clinically might provide further insight to the biological and medical hypothesis of treating cancer with targeted therapies.

### 1.3 Trks and Pain

Besides the newly developed association with cancer, Trks are also being recognized as an important mediator of pain sensation. Congenital insensitivity to pain with anhidrosis (CIPA) is a disorder of the peripheral nerves (and normally innervated sweat glands) that prevents the patient from either being able to adequately perceive painful stimuli or to sweat. TrkA defects have been shown to cause CIPA in various ethnic groups.

Currently, non-steroidal anti-inflammatory drugs (NSAIDs) and opiates have low efficacy and/or side effects (e.g., gastrointestinal/renal and psychotropic side effects, respectively) against neuropathic pain and therefore development of novel pain treatments is highly desired. It has been recognized that NGF levels are elevated in response to chronic pain, injury and inflammation and the administration of exogenous NGF increases pain hypersensitivity. In addition, inhibition of NGF function with either anti-NGF antibodies or non-selective small molecule Trk inhibitors has been shown to have effects on pain in animal models. It appears that a selective Trk inhibitor (inhibiting at least NGF's target, the TrkA receptor) might provide clinical benefit for the treatment of pain. Excellent earlier reviews have covered targeting NGF/BDNF for the treatment of pain so this review will only focus on small molecule Trk kinase inhibitors claimed against cancer and pain. However, it is notable that the NGF antibody tanezumab was very recently reported to show good efficacy in a Phase II trial against osteoarthritic knee pain.”

International Patent Application publication number WO2009/012283 refers to various fluorophenyl compounds as Trk inhibitors; International Patent Application publication numbers WO2009/152087, WO2008/080015 and WO2008/08001 and WO2009/152083 refer to various fused pyrroles as kinase modulators; International Patent Application publication numbers WO2009/143024 and WO2009/143018 refer to various pyrrolo[2,3-d]pyrimidines substituted as Trk inhibitors; International Patent Application publication numbers WO2004/056830 and WO2005/116035 describe various 4-amino-pyrrolo[2,3-d]pyrimidines as Trk inhibitors. International Patent Application publication number WO2011/133637 describes various pyrrolo[2,3-d]pyrimidines and pyrrolo[2,3-b]pyridines as inhibitors of various kinases. International Patent Application publication number WO2005/099709 describes bicyclic heterocycles as serine protease inhibitors. International Patent Application publication number WO2007/047207 describes bicyclic heterocycles as FLAP modulators.

U.S. provisional application 61/471,758 was filed 5 Apr. 2011. Convention applications U.S. Ser. No. 13/439,131 (filed 4 Apr. 2012) and PCT/IB2012/051363 (filed 22 Mar. 2012) claiming priority thereto. The whole contents of those application in their entirety are herewith included by reference thereto.

Thus Trk inhibitors have a wide variety of potential medical uses. There is a need to provide new Trk inhibitors that are good drug candidates. In particular, compounds should preferably bind potently to the Trk receptors in a selective manner compared to other receptors, whilst showing little affinity for other receptors, including other kinase and/or GPC receptors, and show functional activity as Trk receptor antagonists. They should be non-toxic and demonstrate few side-effects. Furthermore, the ideal drug candidate will exist in a physical form that is stable, non-hygroscopic and easily formulated. They should preferably be e.g. well absorbed from the gastrointestinal tract, and/or be injectable directly into the bloodstream, muscle, or subcutaneously, and/or be metabolically stable and possess favourable pharmacokinetic properties.

Among the aims of this invention are to provide orally-active, efficacious, compounds and salts which can be used as active drug substances, particularly Trk antagonists, i.e. that block the intracellular kinase activity of the Trk, e.g. TrkA (NGF) receptor. Other desirable features include good HLM/hepatocyte stability, oral bioavailability, metabolic stability, absorption, selectivity over other types of kinase, dofetilide selectivity. Preferable compounds and salts will show a lack of CYP inhibition/induction, and be CNS-sparing.

### SUMMARY

The present invention provides compounds of Formula I:

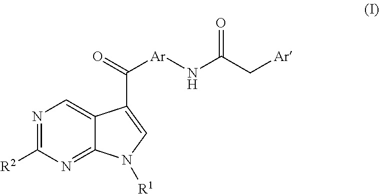

wherein
R¹ is C₂₋₄ alkyl optionally substituted by 1 or 2 OH, optionally wherein a methylene group is replaced by an oxetane group,
or R¹ is C₃₋₆ cycloalkyl optionally substituted by OH,
or R¹ is (C₃₋₆ cycloalkyl)C₁₋₃ alkyl optionally substituted by 1 or 2 OH;

R² is H, OH or NH₂;

Ar is a ring system selected from

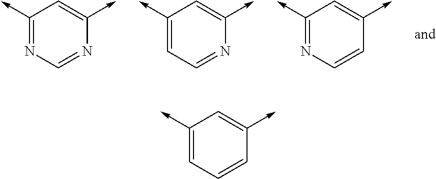

which ring system is optionally substituted on a carbon atom by C₁₋₃ alkyl, CN or C₁₋₃ alkoxy;
and Ar′ is a ring system selected from

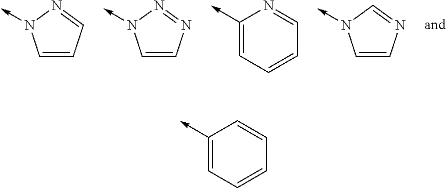

which ring system is optionally substituted on a carbon atom by 1 or 2 substituents independently selected from:
halo, ═O, CN, C₁₋₃ alkyl optionally substituted by one or more F or C₁₋₃ alkoxy, C₁₋₃ alkoxy optionally substituted by one or more F, C₃₋₆ cycloalkyl, C₃₋₆ cycloalkyloxy and SO₂(C₁₋₃ alkyl).
and pharmaceutically acceptable salts thereof.

The invention also comprises pharmaceutical compositions comprising a therapeutically effective amount of a compound of formula I as defined herein, or a pharmaceutically acceptable salt thereof, and a pharmaceutically acceptable carrier.

The invention is also directed to a method of treating a disease or condition indicated for treatment with a Trk antagonist, in a subject, by administering to a subject in need thereof a therapeutically effective amount of one or more of the compounds herein, or a pharmaceutically acceptable salt thereof.

Other aspects of the invention will be apparent from the remaining description and claims.

Preferably, the compounds of the present invention are potent antagonists at Trk receptors, and have a suitable PK profile to enable once daily dosing.

The compounds of the present invention are potentially useful in the treatment of a range of disorders where a Trk antagonist is indicated, particularly pain indications. Depending on the disease and condition of the patient, the term “treatment” as used herein may include one or more of curative, palliative and prophylactic treatment.

According to the invention a compound of the present invention may be useful to treat any physiological pain such as inflammatory pain, nociceptive pain, neuropathic pain, acute pain, chronic pain, musculo-skeletal pain, on-going pain, central pain, heart and vascular pain, head pain, orofacial pain. Other pain conditions which may be treated include intense acute pain and chronic pain conditions which may involve the same pain pathways driven by pathophysiological processes and as such cease to provide a protective mechanism and instead contribute to debilitating symptoms associated with a wide range of disease states.

Pain is a feature of many trauma and disease states. When a substantial injury, via disease or trauma, to body tissue occurs the characteristics of nociceptor activation are altered, this leads to hypersensitivity at the site of damage and in nearby normal tissue. In acute pain the sensitivity returns to normal once the injury has healed. However, in many chronic pain states, the hypersensitivity far outlasts the healing process and is normally due to nervous system injury due to maladaptation of the afferent fibres (Woolf & Salter 2000 Science 288: 1765-1768). Clinical pain is present when discomfort and abnormal sensitivity feature among the patient's symptoms. There are a number of typical pain subtypes: 1) spontaneous pain which may be dull, burning, or stabbing; 2) pain responses to noxious stimuli are exaggerated (hyperalgesia); 3) pain is produced by normally innocuous stimuli (allodynia) (Meyer et al., 1994 Textbook of Pain 13-44). Pain can be divided into a number of different areas because of differing pathophysiology, these include nociceptive, inflammatory, neuropathic pain among others. It should be noted that some types of pain have multiple aetiologies and thus can be classified in more than one area, e.g. Back pain, Cancer pain have both nociceptive and neuropathic components.

### Nociceptive Pain

Nociceptive pain is induced by tissue injury or by intense stimuli with the potential to cause injury. Pain afferents are activated by transduction of stimuli by nociceptors at the site of injury and sensitise the spinal cord at the level of their termination. This is then relayed up the spinal tracts to the brain where pain is perceived (Meyer et al., 1994 Textbook of Pain 13-44). The activation of nociceptors activates two types of afferent nerve fibres. Myelinated A-delta fibres transmit rapidly and are responsible for the sharp and stabbing pain sensations, whilst unmyelinated C fibres transmit at a slower rate and convey the dull or aching pain. Moderate to severe acute nociceptive pain is a prominent feature of, but is not limited to pain from strains/sprains, post-operative pain (pain following any type of surgical procedure), posttraumatic pain, burns, myocardial infarction, acute pancreatitis, and renal colic. Also cancer related acute pain syndromes commonly due to therapeutic interactions such as chemotherapy toxicity, immunotherapy, hormonal therapy and radiotherapy. Moderate to severe acute nociceptive pain is a prominent feature of, but is not limited to, cancer pain which may be tumour related pain, (e.g. bone pain, headache and facial pain, viscera pain) or associated with cancer therapy (e.g. postchemotherapy syndromes, chronic postsurgical pain syndromes, post radiation syndromes), back pain which may be due to herniated or ruptured intervertabral discs or abnormalities of the lumbar facet joints, sacroiliac joints, paraspinal muscles or the posterior longitudinal ligament.

### Neuropathic Pain

According to the invention a compound of the present invention can potentially be used to treat neuropathic pain and the symptoms of neuropathic pain including hyperalgesia, allodynia and ongoing pain. Neuropathic pain is defined as pain initiated or caused by a primary lesion or dysfunction in the nervous system (IASP definition). Nerve damage can be caused by trauma and disease and thus the term ‘neuropathic pain’ encompasses many disorders with diverse aetiologies. These include but are not limited to, Diabetic neuropathy, Post herpetic neuralgia, Back pain, Cancer neuropathy, HIV neuropathy, Phantom limb pain, Carpal Tunnel Syndrome, chronic alcoholism, hypothyroidism, trigeminal neuralgia, uremia, or vitamin deficiencies. Neuropathic pain is pathological as it has no protective role. It is often present well after the original cause has dissipated, commonly lasting for years, significantly decreasing a patients quality of life (Woolf and Mannion 1999 Lancet 353: 1959-1964). The symptoms of neuropathic pain are difficult to treat, as they are often heterogeneous even between patients with the same disease (Woolf & Decosterd 1999 Pain Supp. 6: S141-S147; Woolf and Mannion 1999 Lancet 353: 1959-1964). They include spontaneous pain, which can be continuous, or paroxysmal and abnormal evoked pain, such as hyperalgesia (increased sensitivity to a noxious stimulus) and allodynia (sensitivity to a normally innocuous stimulus).

### Intense Acute Pain and Chronic Pain

Intense acute pain and chronic pain may involve the same pathways driven by pathophysiological processes and as such cease to provide a protective mechanism and instead contribute to debilitating symptoms associated with a wide range of disease states. Pain is a feature of many trauma and disease states. When a substantial injury, via disease or trauma, to body tissue occurs the characteristics of nociceptor activation are altered. There is sensitisation in the periphery, locally around the injury and centrally where the nociceptors terminate. This leads to hypersensitivity at the site of damage and in nearby normal tissue. In acute pain these mechanisms can be useful and allow for the repair processes to take place and the hypersensitivity returns to normal once the injury has healed. However, in many chronic pain states, the hypersensitivity far outlasts the healing process and is normally due to nervous system injury. This injury often leads to maladaptation of the afferent fibres (Woolf & Salter 2000 Science 288: 1765-1768). Clinical pain is present when discomfort and abnormal sensitivity feature among the patient's symptoms. Patients tend to be quite heterogeneous and may present with various pain symptoms. There are a number of typical pain subtypes: 1) spontaneous pain which may be dull, burning, or stabbing; 2) exaggerated pain responses to noxious stimuli (hyperalgesia); 3) pain is produced by normally innocuous stimuli (allodynia) (Meyer et al., 1994 Textbook of Pain 13-44). Although patients with back pain, arthritis pain, CNS trauma, or neuropathic pain may have similar symptoms, the underlying mechanisms are different and, therefore, may require different treatment strategies.

### Chronic Pain

Chronic pain comprises one or more of, chronic nociceptive pain, chronic neuropathic pain, chronic inflammatory pain, breakthrough pain, persistent pain hyperalgesia, allodynia, central sensitisation, peripheral sensitisation, disinhibition and augmented facilitation.

Chronic pain includes cancer pain, e.g. cancer pain arising from malignancy, adenocarcinoma in glandular tissue, blastoma in embryonic tissue of organs, carcinoma in epithelial tissue, leukemia in tissues that form blood cells, lymphoma in lymphatic tissue, myeloma in bone marrow, sarcoma in connective or supportive tissue, adrenal cancer, AIDS-related lymphoma, anemia, bladder cancer, bone cancer, brain cancer, breast cancer, carcinoid tumour s, cervical cancer, chemotherapy, colon cancer, cytopenia, endometrial cancer, esophageal cancer, gastric cancer, head cancer, neck cancer, hepatobiliary cancer, kidney cancer, leukemia, liver cancer, lung cancer, lymphoma, Hodgkin's disease, lymphoma, non-Hodgkin's, nervous system tumours, oral cancer, ovarian cancer, pancreatic cancer, prostate cancer, rectal cancer, skin cancer, stomach cancer, testicular cancer, thyroid cancer, urethral cancer, bone cancer, sarcomas cancer of the connective tissue, cancer of bone tissue, cancer of blood-forming cells, cancer of bone marrow, multiple myeloma, leukaemia, primary or secondary bone cancer, tumours that metastasize to the bone, tumours infiltrating the nerve and hollow viscus, tumours near neural structures. Cancer pain also comprises visceral pain, e.g. visceral pain which arises from pancreatic cancer and/or metastases in the abdomen, somatic pain, e.g. somatic pain due to one or more of bone cancer, metastasis in the bone, postsurgical pain, sarcomas cancer of the connective tissue, cancer of bone tissue, cancer of blood-forming cells of the bone marrow, multiple myeloma, leukaemia, primary or secondary bone cancer.

### Inflammatory Pain

Inflammatory conditions include acute inflammation, persistent acute inflammation, chronic inflammation, and combined acute and chronic inflammation.

Inflammatory pain includes acute inflammatory pain and/or chronic inflammatory pain wherein the chronic inflammatory pain can be pain involving both peripheral and central sensitisation and/or mixed etiology pain involving both inflammatory pain and neuropathic pain or nociceptive pain components. Inflammatory pain also comprises hyperalgesia, e.g. primary and/or secondary hyperalgesia. Additionally or alternatively the inflammatory pain can include allodynia. Inflammatory pain also comprises pain that persists beyond resolution of an underlying disorder or inflammatory condition or healing of an injury.

Inflammatory pain is pain resulting an inflammatory condition. e.g. in response to acute tissue injury due to trauma, disease e.g. an inflammatory disease, immune reaction, the presence of foreign substances, chemicals or infective particles for example micro-organisms. Inflammatory conditions can be either acute or chronic inflammation or both.

Inflammatory pain can result from an inflammatory condition due to an inflammatory disease such as inflammatory joint diseases, inflammatory connective tissue diseases, inflammatory autoimmune diseases, inflammatory myopathies, inflammatory digestive system diseases, inflammatory air way diseases, cellular immune inflammation diseases, hypersensitivities and allergies, vasular inflammation diseases, non-immune inflammatory disease, synovitis, villonodular synovitis, arthralgias, ankylosing spondylitis, spondyloarthritis, spondyloarthropathy, gout, Pagets disease, periarticular disorders such as bursitis, rheumatoid disease, rheumatoid arthritis and osteoarthritis, rheumatoid arthritis or osteoarthritis. Rheumatoid arthritis in particular, represents ongoing inflammation associated with severe pain. Arthritic pain is a form of inflammatory pain and arises from inflammation in a joint which causes both peripheral sensitization and central sensitization. Under inflammatory conditions the nociceptive system is activated by normally innocuous and nonpainful mechanical stimuli. Additionally when the joint is at rest pain is present and appears as spontaneous pain and hyperalgesia (augmented pain response on noxious stimulation and pain on normally nonpainful stimulation). Inflammatory processes in peripheral tissues lead to central sensitization in the spinal cord, which contributes to hyperalgesia and allodynia typically associated with inflammatory pain. Other types of inflammatory pain include inflammatory bowel diseases (IBD).

### Other Types of Pain

Other types of pain include but are not limited to:
Musculo-skeletal disorders including but not limited to myalgia, fibromyalgia, spondylitis, sero-negative (non-rheumatoid) arthropathies, non-articular rheumatism, dystrophinopathy, Glycogenolysis, polymyositis, pyomyositis;
Central pain or ‘thalamic pain’ as defined by pain caused by lesion or dysfunction of the nervous system including but not limited to central post-stroke pain, multiple sclerosis, spinal cord injury, Parkinson's disease and epilepsy;
Heart and vascular pain including but not limited to angina, myocardical infarction, mitral stenosis, pericarditis, Raynaud's phenomenon, scleredoma, scleredoma, skeletal muscle ischemia;
Visceral pain, and gastrointestinal disorders. The viscera encompasses the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders include the functional bowel disorders (FBD) and the inflammatory bowel diseases (IBD). These GI disorders include a wide range of disease states that are currently only moderately controlled, including—for FBD, gastro-esophageal reflux, dyspepsia, the irritable bowel syndrome (IBS) and functional abdominal pain syndrome (FAPS), and—for IBD, Crohn's disease, ileitis, and ulcerative colitis, which all regularly produce visceral pain. Other types of visceral pain include the pain associated with dysmenorrhea, pelvic pain, cystitis and pancreatitis;

Head pain including but not limited to migraine, migraine with aura, migraine without aura cluster headache, tension-type headache. Orofacial pain including but not limited to dental pain, temporomandibular myofascial pain, tinnitus, hot flushes, restless leg syndrome and blocking development of abuse potential. Further pain conditions may include, back pain (e.g. chronic lower back pain), cancer pain, complex regional syndrome, HIV-related neuropathic pain, post-operative induced neuropathic pain, post-stroke pain, spinal cord injury pain, traumatic nerve injury pain, diabetic peripheral neuropathy, moderate/severe interstitial cystitis pain, irritable bowel syndrome pain, moderate/severe endometriosis pain, moderate/severe pelvic pain, moderate/severe prostatitis pain, moderate/severe osteoarthritis pain, post-herpetic neuralgia, rheumatoid arthritis pain, dysmenorrhea pain, pre-emptive post-operative pain, trigeminal neuralgia, bursitis, dental pain, fibromyalgia or myofacial pain, menstrual pain, migraine, neuropathic pain (including painful diabetic neuropathy), pain associated with post-herpetic neuralgia, post-operative pain, referred pain, trigeminal neuralgia, visceral pain (including interstitial cystitis and IBS) and pain associated with AIDS, allodynia, burns, cancer, hyperalgesia, hypersensitisation, spinal trauma and/or degeneration and stroke.

### DETAILED DESCRIPTION

Embodiment 1 of the invention is a compound of Formula I:

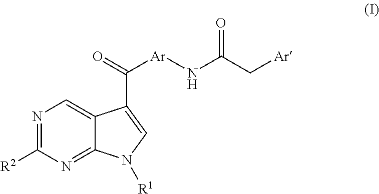

or a pharmaceutically acceptable salt thereof, wherein
R¹ is C₂₋₄ alkyl optionally substituted by 1 or 2 OH, optionally wherein a methylene group is replaced by an oxetane group,
or R¹ is C₃₋₆ cycloalkyl optionally substituted by OH,
or R¹ is (C₃₋₆ cycloalkyl)C₁₋₃ alkyl optionally substituted by 1 or 2 OH;

### R² is H, OH or NH₂;

Ar is a ring system selected from

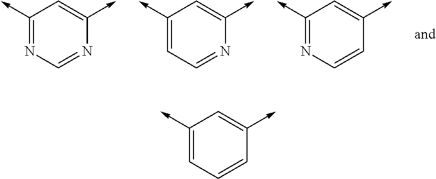

which ring system is optionally substituted on a carbon atom by C₁₋₃ alkyl, CN or C₁₋₃ alkoxy;
and Ar′ is a ring system selected from

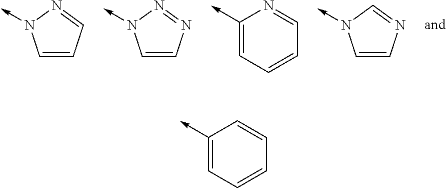

which ring system is optionally substituted on a carbon atom by 1 or 2 substituents independently selected from:
halo, ═O, CN, C₁₋₃ alkyl optionally substituted by one or more F or C₁₋₃ alkoxy, C₁₋₃ alkoxy optionally substituted by one or more F, C₃₋₆ cycloalkyl, C₃₋₆ cycloalkyloxy and SO₂(C₁₋₃ alkyl).

### Embodiment 2

A compound or salt according to embodiment 1 wherein R¹ is selected from:

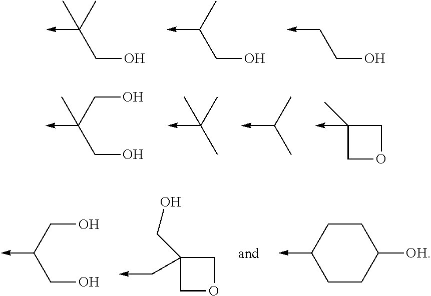

### Embodiment 3

A compound or salt according to embodiment 1 or 2 wherein Ar′ is a ring system selected from

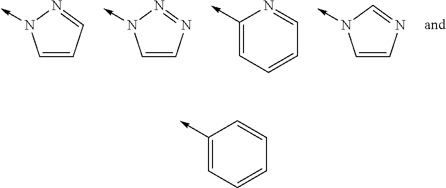

which ring system is substituted on a carbon atom by 1 or 2 substituents independently selected from:
F, Cl, ═O, CN, CH₃, CF₃, OCF₃, cyclopropyl, cyclopropyloxy and SO₂CH₃.

### Embodiment 4

A compound or salt according to embodiment 1, 2 or 3 wherein R¹ is selected from

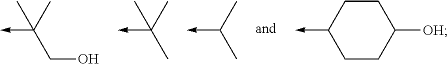

### R² is H or NH₂;

Ar is selected from

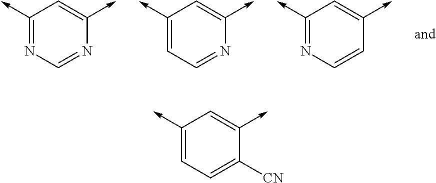

and Ar′ is a ring system selected from

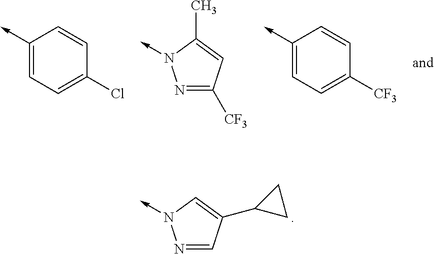

### Embodiment 5

A compound selected from the compounds of Examples 1, 7, 36, 44, 47, 48, 65, 147, or a pharmaceutically acceptable salt thereof.

### Embodiment 6

A pharmaceutical composition comprising a compound of the formula (I) or a pharmaceutically acceptable salt thereof, as defined in any one of the preceding embodiments 1 to 5, and a pharmaceutically acceptable carrier.

### Embodiment 7

A compound of the formula (I) or a pharmaceutically acceptable salt thereof, as defined in any one of embodiments 1 to 5, for use as a medicament.

### Embodiment 8

A compound of formula (I) or a pharmaceutically acceptable salt thereof, as defined in any one of embodiments 1 to 5 for use in the treatment of a disease for which a Trk receptor antagonist is indicated.

### Embodiment 9

A compound of formula (I) or a pharmaceutically acceptable salt thereof, as defined in any one of embodiments 1 to 5 for use in the treatment of pain or cancer.

### Embodiment 10

The use of a compound of the formula (I) or a pharmaceutically acceptable salt or composition thereof, as defined in any one of embodiments 1 to 5, for the manufacture of a medicament to treat a disease for which a Trk receptor antagonist is indicated

### Embodiment 11

The use of a compound of the formula (I) or a pharmaceutically acceptable salt or composition thereof, as defined in any one of embodiments 1 to 5, for the manufacture of a medicament to treat pain or cancer.

### Embodiment 12

A method of treatment of a mammal, to treat a disease for which an Trk receptor antagonist is indicated, comprising treating said mammal with an effective amount of a compound of the formula (I) or a pharmaceutically acceptable salt thereof, as defined in any one of embodiments 1 to 5.

### Embodiment 13

A method of treatment of pain or cancer in a mammal, comprising treating said mammal with an effective amount of a compound of the formula (I) or a pharmaceutically acceptable salt thereof, as defined in any one of embodiments 1 to 5.

### Embodiment 14

A compound or salt according to any one of embodiments 1 to 5 for use in a medical treatment in combination with a further drug substance.

### Further Embodiments Include:

A compound or salt according to any one of embodiments wherein R¹ is selected from

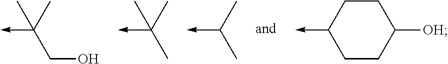

A compound or salt according to any one of embodiments wherein Ar is selected from

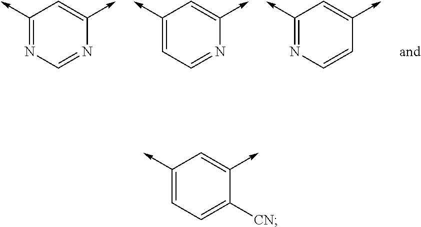

A compound or salt according to any one of embodiments wherein Ar′ is a ring system selected from

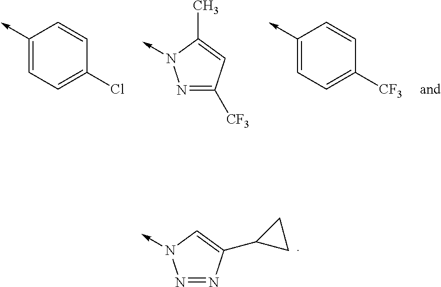

A compound or salt according to any one of embodiments wherein Ar has the value of Ar in any of the Examples;

A compound or salt according to any one of embodiments wherein Ar′ has the value of Ar′ in any of the Examples;

A compound or salt according to any one of embodiments wherein R¹ has the value of R¹ in any of the Examples;

A compound or salt according to any one of embodiments wherein R² has the value of R² in any of the Examples;

A compound selected from any of the Examples herein described, or a pharmaceutically acceptable salt thereof;

Any novel genus of intermediates described in the Schemes below;

Any novel specific intermediate described in the Preparations below;

Any novel process described herein.

“Halogen” means a fluoro, chloro, bromo or iodo group.

“Alkyl” groups, containing the requisite number of carbon atoms, can be unbranched or branched. Examples of alkyl include methyl, ethyl, n-propyl, i-propyl, n-butyl, i-butyl, sec-butyl and t-butyl.

“Pharmaceutically acceptable salts” of the compounds of formula I include the acid addition and base addition salts (including disalts, hemisalts, etc.) thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base addition salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002).

The compounds of the invention include compounds of formula I and salts thereof as hereinbefore defined, polymorphs, and isomers thereof (including optical, geometric and tautomeric isomers) as hereinafter defined and isotopically-labelled compounds of formula I.

Unless otherwise specified, compounds of formula (I) containing one or more asymmetric carbon atoms can exist as two or more stereoisomers. Where a compound of formula (I) contains for example, a keto or guanidine group or an aromatic moiety, tautomeric isomerism (‘tautomerism’) can occur. It follows that a single compound may exhibit more than one type of isomerism.

Included within the scope of the claimed compounds of the present invention are all stereoisomers, geometric isomers and tautomeric forms of the compounds of formula (I), including compounds exhibiting more than one type of isomerism, and mixtures of one or more thereof. Also included are acid addition or base addition salts wherein the counterion is optically active, for example, D-lactate or L-lysine, or racemic, for example, DL-tartrate or DL-arginine.

Examples of types of potential tautomerisms shown by the compounds of the invention include hydroxypyridine [symbol] pyridone; amide [symbol] hydroxyl-imine and keto [symbol] enol tautomersims:

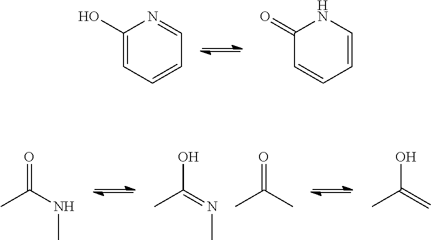

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or other derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Mixtures of stereoisomers may be separated by conventional techniques known to those skilled in the art. [see, for example, “Stereochemistry of Organic Compounds” by E L Eliel (Wiley, New York, 1994).]

The present invention includes all pharmaceutically acceptable isotopically-labelled compounds of formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds of formula (I) can generally be prepared by conventional techniques known to those skilled in the art or by processes analogous to those described in the accompanying Examples and Preparations using an appropriate isotopically-labelled reagents in place of the non-labelled reagent previously employed.

The routes below, including those mentioned in the Examples and Preparations, illustrate methods of synthesising compounds of formula (I). The skilled person will appreciate that the compounds of the invention, and intermediates thereto, could be made by methods other than those specifically described herein, for example by adaptation of the methods described herein, for example by methods known in the art. Suitable guides to synthesis, functional group interconversions, use of protecting groups, etc., are for example: “Comprehensive Organic Transformations” by R C Larock, VCH Publishers Inc. (1989); Advanced Organic Chemistry” by J. March, Wiley Interscience (1985); “Designing Organic Synthesis” by S Warren, Wiley Interscience (1978); “Organic Synthesis—The Disconnection Approach” by S Warren, Wiley Interscience (1982); “Guidebook to Organic Synthesis” by R K Mackie and D M Smith, Longman (1982); “Protective Groups in Organic Synthesis” by T W Greene and PGM Wuts, John Wiley and Sons, Inc. (1999); and “Protecting Groups” by P J, Kocienski, Georg Thieme Verlag (1994); and any updated versions of said standard works.

In addition, the skilled person will appreciate that it may be necessary or desirable at any stage in the synthesis of compounds of the invention to protect one or more sensitive groups, so as to prevent undesirable side reactions. In particular, it may be necessary or desirable to protect amino or carboxylic acid groups. The protecting groups used in the preparation of the compounds of the invention may be used in conventional manner. See, for example, those described in ‘Greene's Protective Groups in Organic Synthesis’ by Theodora W Greene and Peter G M Wuts, third edition, (John Wiley and Sons, 1999), in particular chapters 7 (“Protection for the Amino Group”) and 5 (“Protection for the Carboxyl Group”), incorporated herein by reference, which also describes methods for the removal of such groups.

In the general synthetic methods below, unless otherwise specified, the substituents are as defined above with reference to the compounds of formula (I) above.

Where ratios of solvents are given, the ratios are by volume.

According to a first process, compounds of formula (I) may be prepared by the process illustrated in Scheme 1.

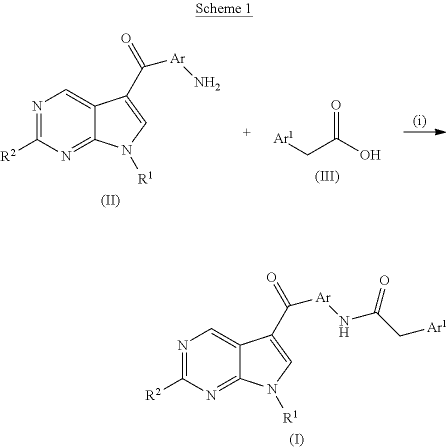

Compounds of formula (I) may be prepared from compounds of formula (II) according to process step (i), an amide bond formation step, if necessary adding a suitable base (such as DIPEA) and/or additive (such as DMAP).

Typical conditions employed involve stirring the amine of general formula (II) and the acid of general formula (III) together with a suitable coupling reagent such as HATU or 1-propylphosphonic acid cyclic anhydride, if necessary adding a suitable base such as NMM, DIPEA or TEA in a suitable solvent such as pyridine, THF or DMA at a temperature from room temperature up to 50° C. A suitable alternative is to use an additive (such as 4-dimethylaminopyridine) as well as a base. Any suitable solvent may be used in place of those mentioned above. At least one equivalent of the acid (III) and at least one equivalent of the coupling reagent should be used and an excess of one or both may be used if desired.

Where R¹ contains a suitable hydroxyl protecting group in intermediate (II), removal of the protecting group (PG) can be done in situ or as an additional step, adding a suitable acid and organic solvent to the crude residue after the amide bond formation has taken place. Common protecting groups to use include TBDMS or TMS, which are readily removed by treatment with an acid such as aqueous hydrogen chloride or hydrogen chloride in an organic solvent such as THF or dioxane or by treatment with a fluoride source such as tetrabutylammonium fluoride in an organic solvent such as THF, and THP and dimethylacetal. Preferred conditions comprise HCl in 1,4-dioxane at room temperature.

Intermediates of general formula (III) are either commercially available or will be well-known to those skilled in the art with reference to literature precedents and/or the preparations herein.

Compounds of general formula (II) are described in Schemes 6, 7 and 8.

According to a second process, where Ar is pyrimidine, compounds of formula (I) may be prepared by the process illustrated in Scheme 2.

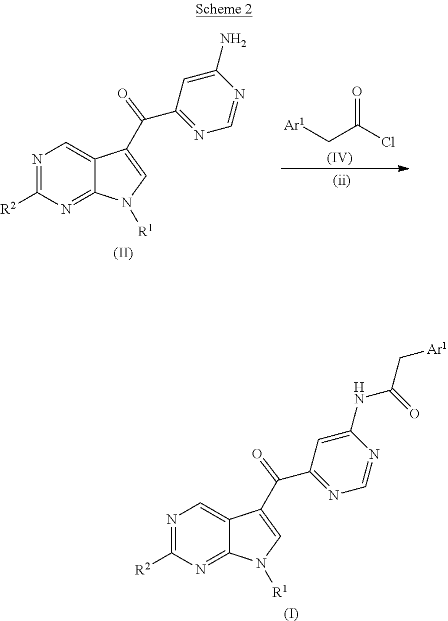

Compounds of formula (I) may be prepared from compounds of formula (II) and (IV) according to process step (ii), an amide bond forming step in the presence of a strong organometallic base, such as NaHMDS, and an acid chloride. Preferred conditions comprise NaHMDS in THF at from 0° C. to room temperature.

Intermediates of general formula (IV) are either commercially available or will be well-known to those skilled in the art with reference to literature precedents and/or the preparations herein.

Compounds of general formula (II) are described in Schemes 6 and 7.

According to a third process, when Ar¹ is CN-substituted pyridyl, compounds of formula (I) may be prepared by the process illustrated in Scheme 3.

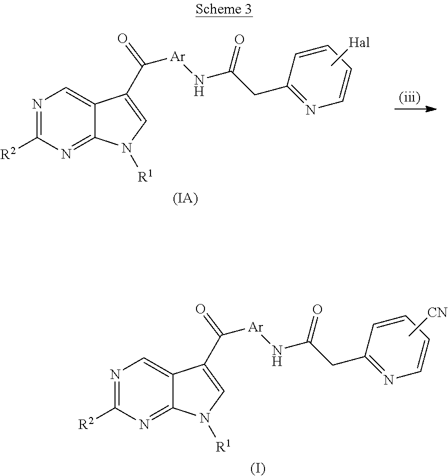

Compounds of formula (I) may be prepared from compounds of formula (IA) according to process step (iii), a palladium catalysed cyanation step. Typical conditions comprise zinc cyanide with tris(dibenzylideneacetone)dipalladium (0) and DPPF in DMF at 100° C. under microwave irradiation.

Compounds of formula (IA) may be prepared as described in Scheme 1.

According to a fourth process, when Ar is alkoxypyridyl, compounds of formula (I) may be prepared by the process illustrated in Scheme 4.

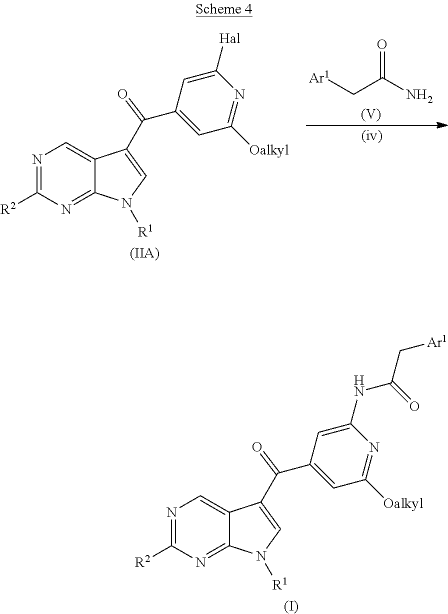

Wherein Hal is Cl, Br or I;

Compounds of formula (I) may be prepared from compounds of formula (IIA) and amides of formula (V) according to process step (iv), a palladium catalysed Buchwald C-N cross coupling reaction. Preferred conditions comprise tris(dibenzylideneacetone)dipalladium (0) with xantphos and cesium carbonate in a suitable organic solvent such as DMF at 120° C.

Compounds of formula (IIA) may be prepared by analogy to the process as described in Schemes 6 and 7.

Compounds of general formula (V) are either commercially available or will be well-known to those skilled in the art with reference to literature precedents and/or the preparations herein.

According to a fifth process, compounds of formula (I) may be prepared by the process illustrated in Scheme 5.

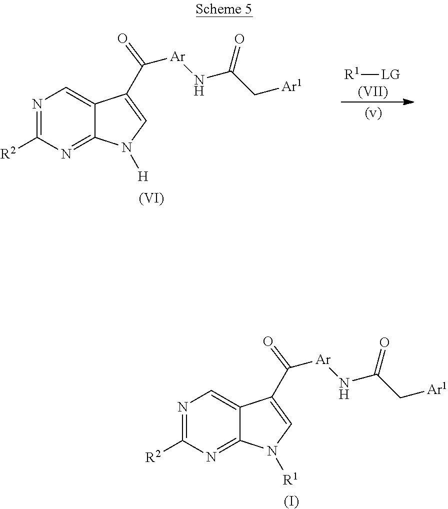

Wherein LG is a leaving group such as Cl, Br or I or an intramolecular leaving group (e.g. exemplified in Preparation 122);

Compounds of formula (I) may be prepared from compounds of formula (VI) and (VII) according to reaction step (v), an alkylation step in the presence of an inorganic base.

Typical conditions comprise potassium carbonate in DMF at room temperature.

Compounds of general formula (VI) may be prepared as described for analogous compounds of general formula (I) in Scheme 1.

Compounds of formula (VII) are either commercially available, or wherein LG is an intramolecular LG, are described by the preparations herein.

According to a sixth process, compounds of formula (II) may be prepared by the process illustrated in Scheme 6.

NB R^{2A} can take the same meaning as R² or can also be a precursor to R² such as a Cl in certain reactions.

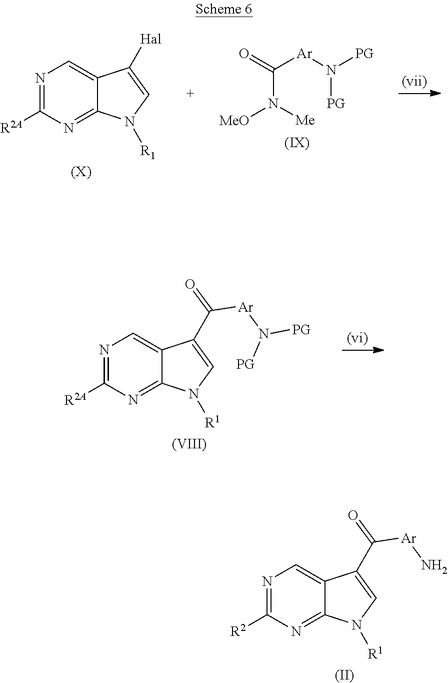

Wherein Hal is Cl, Br or I; PG is a suitable protecting group such as diphenylmethylene; Compounds of formula (II) may be prepared from compounds of formula (VIII) according to process step (vi), a deprotection step conveniently mediated under acidic conditions using acids such as HCl, TFA or citric acid. Wherein PG is diphenylmethylene, preferred conditions comprise a 1M aqueous solution of citric acid in THF at room temperature, neat TFA at room temperature or 1M HCl in THF at room temperature.

Wherein R^{2A} is CI, the intermediate (VIII) may be stirred with a stronger acid such as 6N HCl at 80° C. to afford the hydroxyl intermediate (VIII) wherein R^{2A} is OH at the same time as the amino deprotection.

Compounds of formula (VIII) may be prepared from compounds of formula (X) and (IX) according to process step (vii), a metallation of intermediate halide (X) (using a suitable organometallic reagent such as butyllithium or isopropylmagnesium chloride) and reacting with the Weinreb amide intermediate (IX) at a temperature from −78° C. up to room temperature in a suitable solvent such as THF.

Preferred conditions comprise nBuLi in THF at −78° C. or iPrMgCl in THF at 0° C.

Compounds of formula (X) are described in Schemes 9 and 10. Compounds of formula (IX) are either commercially available or will be well-known to those skilled in the art with reference to literature precedents and/or the preparations herein. Compounds of formula (IIA) described in Scheme 4 may be prepared according to an analogous process as described in Scheme 6.

According to a seventh process, compounds of formula (II) may be prepared by the process illustrated in Scheme 7.

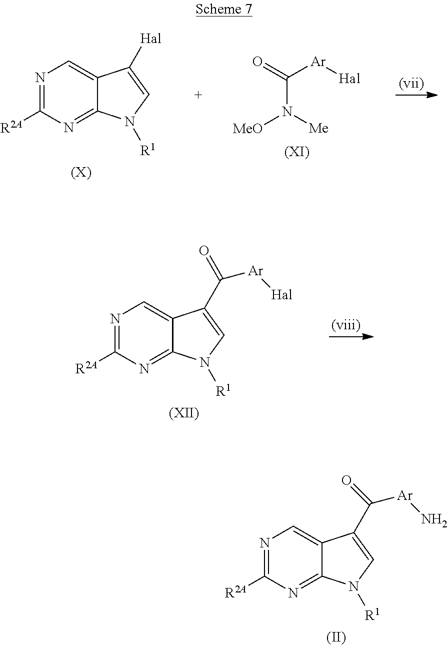

Compounds of formula (II) may be prepared from compounds of formula (XII) according to process step (viii), a direct amination of the halide using standard literature conditions. For example, amine (II) is typically prepared using ammonia with a suitable copper catalyst such as copper (II) sulphate or copper (I) oxide in a suitable solvent such as NMP in a sealed vessel at a temperature between room temperature and 140° C.

Compounds of formula (XII) may be prepared from compounds of formula (X) and (XI) according to analogous process step (vii) as described in Scheme 6.

Wherein R^{2A} is Cl, this too may undergo displacement to afford R² as NH₂ directly. Preferred conditions comprise 880 ammonia and formaldehyde in NMP at 140° C.

Compounds of formula (XI) are either commercially available or will be well-known to those skilled in the art with reference to literature precedents and/or the preparations herein.

Compounds of formula (X) are described in Schemes 9 and 10.

According to an eighth process, wherein Ar is CN-substituted phenyl, compounds of formula (II) may be prepared by the process illustrated in Scheme 8.

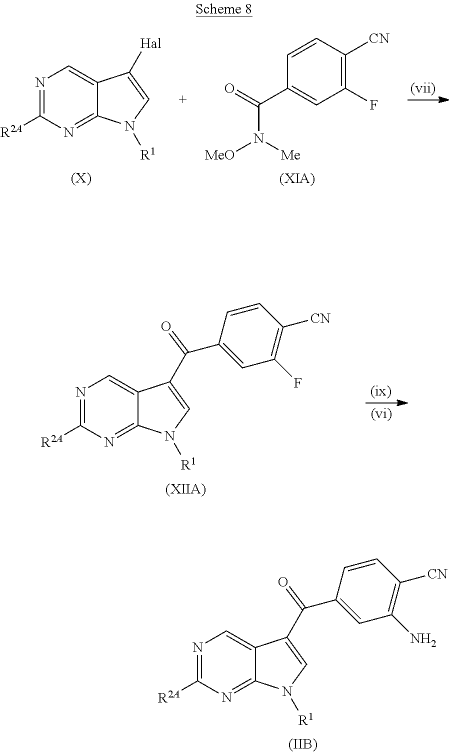

Compounds of formula (IIB) may be prepared from compounds of formula (XIIA) according to process step (ix), an SnAr displacement reaction with 4-methoxybenzylamine followed by an acid mediated deprotection step (vi). Preferred conditions comprise 4-methoxybenzylamine in DMSO at 100° C. followed by neat TFA. Wherein R^{2A} is CI, two equivalents of 4-methoxybenzylamine may be used followed by deprotection as described to afford (IIB) wherein R^{2A} is NH₂ in the same step.

Compounds of formula (XIIA) may be prepared according to reaction step (vii) as described in Scheme 6.

Compounds of formula (XIA) are either commercially available or will be well-known to those skilled in the art with reference to literature precedents and/or the preparations herein.

Compounds of formula (X) are described in Schemes 9 and 10.

According to a ninth process, compounds of formula (X) may be prepared by the process illustrated in Scheme 9.

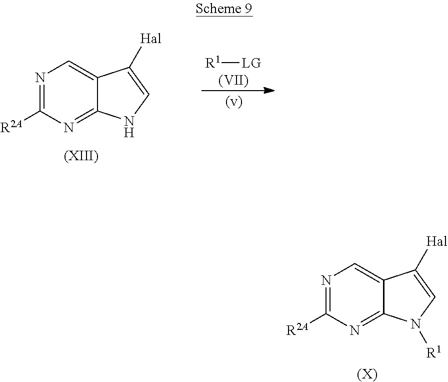

Wherein LG is halogen or tosylate, triflate or mesylate;

Intermediates of general formula (X) can be prepared by alkylation of the pyrrolopyrimidine intermediates (XIII), with compounds of formula (VII) using a suitable inorganic base such as cesium carbonate or potassium carbonate either with or without potassium iodide, in an organic solvent as described in Scheme 5.

Compounds of formula (XIII) are commercially available or well know to those skilled in the art with reference to literature procedures.

In those cases where R¹ contains one or more alcohols, a protected form of R¹ can be used as described in Scheme 1.

According to a tenth process, compounds of formula (X) may be prepared by the process illustrated in Scheme 10.

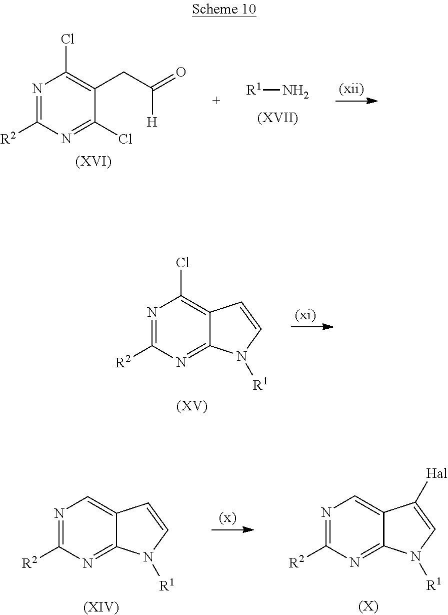

Compounds of formula (X) may be prepared from compounds of formula (XIV) according to process step (x) an electrophilic halogenation reaction. Typical conditions comprise NIS or NBS in acetonitrile at room temperature.

Compounds of formula (XIV) may be prepared from compounds of formula (XV), according to process step (xi), a dechlorination step. The intermediate chloride (XV) is reduced using standard literature conditions, for example hydrogenation using a suitable catalyst such as palladium on carbon and an additive such as ammonia in a suitable solvent such as ethanol.

Compounds of formula (XV) may be prepared from compounds of formula (XVI) and (XVII), according to reaction step (xii), a cyclisation step. Typical conditions employed involve stirring the amine of general formula (XVII) and the aldehyde (XVI) together, preferably in ethanol at a temperature from room temperature up to 80° C. Alternatively, a protected form of the aldehyde may be used, such as 4,6-dichloro-5-(2,2-diethoxyethyl)pyrimidine.

In those cases where R¹ contains one or more alcohols, a protected form of R¹ with a suitable hydroxyl protecting group (PG) can be used. Any suitable oxygen protecting group protection/deprotection system may be used (as described in “Protecting Groups in Organic Synthesis” 3^{rd} edition T. W. Greene and P. G. Wuts, Wiley-Interscience, 1999). Common oxygen protecting groups suitable for use herein include tert-butyldimethylsilyl (TBDMS) and tetrahydropyranyl (THP) and trimethylsilyl (TMS). The hydroxyls herein may be protected and re-protected as necessary according to standard literature conditions.

Intermediates of general formula (XVI) and (XVII) are either commercially available or will be well-known to those skilled in the art with reference to literature precedents and/or the preparations herein.

According to an eleventh process, compounds of formula (XA), (XB) and (XC) may be prepared by the process illustrated in Scheme 11.

NB R^{2B} is H or Cl.

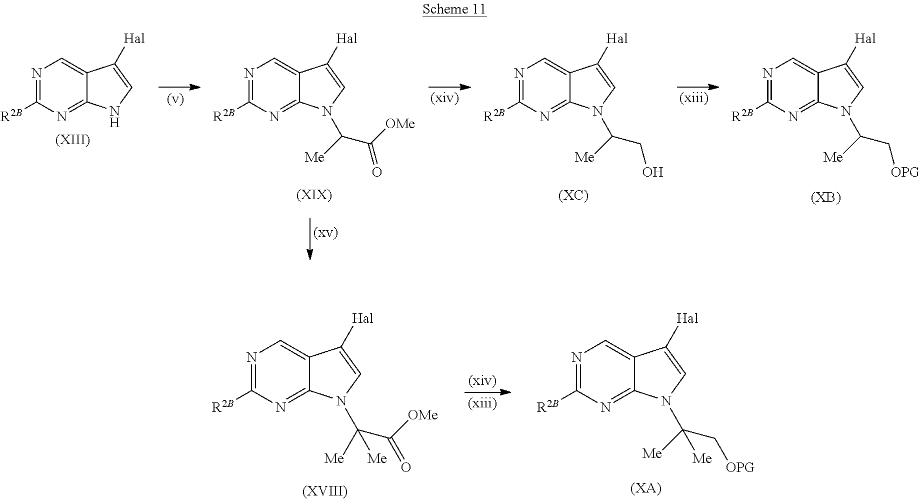

Compounds of formula (XB) may be prepared from compounds of formula (XC) using process step (xiii), a protection step. As previously mentioned in Scheme 1 the hydroxy group can be protected with a suitable oxygen protecting group (PG), where the preferred protecting groups are TBDMS, TMS and THP. Typical conditions comprise TBDMSCI in DCM at 0° C. with imidazole.

Compounds of formula (XC) may be prepared from compounds of formula (XIX) according to process step (xiv), a reduction step. Reduction of the ester intermediate (XIX) can be effected by using a suitable reducing reagent such as lithium borohydride, lithium alumninium hydride or diisobutylalumnium hydride in a suitable solvent such as ethanol or THF. Preferred conditions comprise lithium borohydride in THF at 0° C. or sodium borohydride in EtOH at room temperature.

Compounds of formula (XA) may be prepared from compounds of formula (XVIII) according to process steps (xiv) and (xiii) as described above.

Compounds of formula (XVIII) may be prepared from compounds of formula (XIX) according to process step (xv), a further alkylation step. Typical conditions comprise 1M potassium tertbutoxide in THF at room temperature with an suitable alkylating agent such as methyl iodide.

Compounds of formula (XIX) may be prepared from compounds of formula (XIII) according to analogous process step (v), an alkylation step as described in Scheme 5.

Compounds of formula (XIII) are commercially available or well know to those skilled in the art with reference to literature procedures.

According to an twelfth process, compounds of formula (XD), may be prepared by the process illustrated in Scheme 12.

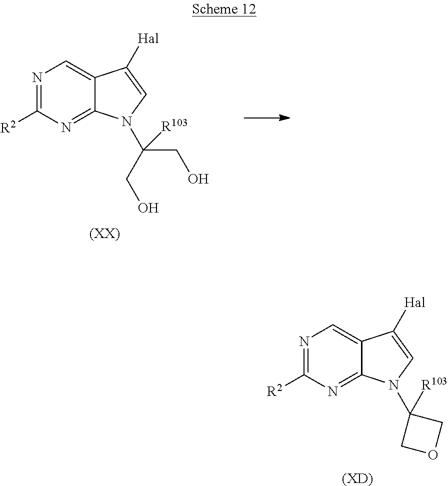

Wherein R¹⁰³ is Me

Compounds of formula (XD) can be prepared from compounds of formula (XX) through conversion of an alcohol into a suitable leaving group followed by cyclisation under basic conditions. Preferred conditions comprise tosyl chloride with n-butyl lithium in THF.

According to a further embodiment the present invention provides novel intermediate compounds.

Pharmaceutically acceptable salts of a compound of formula (I) may be readily prepared by mixing together solutions of the compound of formula (I) and the desired acid or base, as appropriate. The salt may precipitate from solution and be collected by filtration or may be recovered by evaporation of the solvent. The degree of ionisation in the salt may vary from completely ionised to almost non-ionised.

The compounds of the invention intended for pharmaceutical use may be administered alone or in combination with one or more other compounds of the invention or in combination with one or more other drug agent (or as any combination thereof). Generally, they will be administered as a formulation in association with one or more pharmaceutically acceptable excipients. The term “excipient” is used herein to describe any biologically inactive ingredient other than the compounds and salts of the invention. The choice of excipient will to a large extent depend on factors such as the particular mode of administration, the effect of the excipient on solubility and stability, and the nature of the dosage form. For example, a compound of the formula I, or a pharmaceutically acceptable salt or solvate thereof, as defined above, may be administered simultaneously (e.g. as a fixed dose combination), sequentially or separately in combination with one or more other drug agent.

Exemplary additional agents could be selected from one or more of:
a Nav1.7 channel modulator, such as a compound disclosed in WO 2009/012242 or WO2010/079443;
an alternative sodium channel modulator, such as a Nav1.3 modulator (e.g. as disclosed in WO2008/118758); or a Nav1.8 modulator (e.g. as disclosed in WO 2008/135826, more particularly N-[6-Amino-5-(2-chloro-5-methoxyphenyl)pyridin-2-yl]-1-methyl-1H-pyrazole-5-carboxamide);
an inhibitor of nerve growth factor signaling, such as: an agent that binds to NGF and inhibits NGF biological activity and/or downstream pathway(s) mediated by NGF signaling (e.g. tanezumab), a TrkA antagonist or a p75 antagoinsist;
a compound which increases the levels of endocannabinoid, such as a compound with fatty acid amid hydrolase inhibitory (FAAH) activity, in particular those disclosed in WO 2008/047229 (e.g. N-pyridazin-3-yl-4-(3-{[5-(trifluoromethyl)pyridine-2-yl]oxy}benzylidene)piperidene-1-carboxamide);
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butabital, mephobarbital, metharbital, methohexital, pentobarbital, phenobartital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorcyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphrenadine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl) quinazoline;
a tricyclic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3, NK-2 or NK-1 antagonist, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g oxybutynin, tolterodine, propiverine, tropsium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT_{1B/1D} agonist such as eletriptan, sumatriptan, naratriptan, zolmitriptan or rizatriptan;
a 5-HT_{2A} receptor antagonist such as R(+)-alpha-(2,3-dimethoxy-phenyl)-1-[2-(4-fluorophenylethyl)]-4-piperidinemethanol (MDL-100907);
a 5-HT₃ antagonist, such as ondansetron
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
Tramadol®;
a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-Nyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
an alpha-2-delta ligand such as gabapentin, pregabalin, 3-methylgabapentin, (1α,3α,5α)(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan®), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl)butyl]thio]-6-(trifluoromethyl)-3 pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxyl)pyridin-3-yl]carbonyl}amino)ethyl]benzoic acid;
a microsomal prostaglandin E synthase type 1 (mPGES-1) inhibitor;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,
   a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl), 1,4-benzoquinone (CV-6504).

Pharmaceutical compositions suitable for the delivery of compounds and salts of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

Compounds and salts of the invention intended for pharmaceutical use may be prepared and administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

### Oral Administration

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations, such as tablets, capsules containing particulates, liquids, or powders; lozenges (including liquid-filled), chews; multi- and nano-particulates; gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavoring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant. [Make sure these specific ranges are relevant]

Tablet blends may be compressed directly or by roller to form tablets. Tablet blends or portions of blends may alternatively be wet-, dry-, or melt-granulated, melt congealed, or extruded before tableting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

The foregoing formulations for the various types of administration discussed above may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO 00/35298.

### Parenteral Administration

The compounds and salts of the invention may be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) and salts used in the preparation of parenteral solutions may be increased by the use of appropriate formulation techniques, such as the incorporation of solubility-enhancing agents.

Formulations for parenteral administration may be formulated to be immediate and/or modified release. Thus, compounds and salts of the invention may be formulated as a solid, semi-solid, or thixotropic liquid for administration as an implanted depot providing modified release of the active compound. An example of such formulations include drug-coated stents.

### Topical Administration

The compounds and salts of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated [see, for example, Finnin and Morgan, J Pharm Sci, 88 (10), 955-958 (October 1999).] Other means of topical administration include delivery by electroporation, iontophoresis, phonophoresis, sonophoresis and microneedle or needle-free (e.g. Powderject™, Bioject™, etc.) injection.

### Inhaled/Intranasal Administration

The compounds and salts of the invention may also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

A pressurised container, pump, spray, atomizer, or nebuliser may contain a solution or suspension of the compound(s) or salt(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or HPMC), blisters and cartridges for use in an inhaler or insufflator may be formulated to contain a powder mix of the compound or salt of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound or salt of the invention per actuation and the actuation volume may vary from 1 μl to 100 μl. A typical formulation may comprise a compound of formula (I) or salt thereof, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by a prefilled capsule, blister or pocket or by a system that utilises a gravimetrically fed dosing chamber. Units in accordance with the invention are typically arranged to administer a metered dose or “puff” containing from 1 to 5000 μg of the compound or salt. The overall daily dose will typically be in the range 1 μg to 20 mg which may be administered in a single dose or, more usually, as divided doses throughout the day.

### Rectal/Intravaginal Administration

The compounds and salts of the invention may be administered rectally or vaginally, for example, in the form of a suppository, pessary, or enema. Cocoa butter is a traditional suppository base, but various well known alternatives may be used as appropriate.

### Ocular and Aural Administration

The compounds and salts of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid; a cellulosic polymer, for example, hydroxypropylmethylcellulose, hydroxyethylcellulose, or methyl cellulose; or a heteropolysaccharide polymer, for example, gelan gum, may be incorporated together with a preservative, such as benzalkonium chloride. Such formulations may also be delivered by iontophoresis.

### Other Technologies

The compounds and salts of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

For administration to human patients, the total daily dose of the compounds and salts of the invention is typically in the range 0.1 mg to 200 mg depending, of course, on the mode of administration, preferred in the range 1 mg to 100 mg and more preferred in the range 1 mg to 50 mg. The total daily dose may be administered in single or divided doses.

These dosages are based on an average human subject having a weight of about 65 kg to 70 kg. The physician will readily be able to determine doses for subjects whose weight falls outside this range, such as infants and the elderly.

For the above-mentioned therapeutic uses, the dosage administered will, of course, vary with the compound or salt employed, the mode of administration, the treatment desired and the disorder indicated. The total daily dosage of the compound of formula (I)/salt/solvate (active ingredient) will, generally, be in the range from 1 mg to 1 gram, preferably 1 mg to 250 mg, more preferably 10 mg to 100 mg. The total daily dose may be administered in single or divided doses. The present invention also encompasses sustained release compositions.

The pharmaceutical composition may, for example, be in a form suitable for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regiments for administration of the chemotherapeutic agent are well-known in the relevant art and would be understood to be encompassed by the skilled artisan once provided the teachings disclosed herein.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

For parenteral dosages, this may conveniently be prepared as a solution or as a dry powder requiring dissolution by a pharmacist, medical practitioner or the patient. It may be provided in a bottle or sterile syringe. For example it may be provided as a powder in a multicompartment syringe which allows the dry powder and solvent to be mixed just prior to administration (to aid long-term stability and storage). Syringes could be used which allow multiple doses to be administered from a single device.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations as discussed below. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

A composition of the present invention can be administered by a variety of methods known in the art. The route and/or mode of administration vary depending upon the desired results. The active compounds can be prepared with carriers that protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., Sustained and Controlled Release Drug Delivery Systems, J. R. Robinson, ed., Marcel Dekker, Inc., New York, (1978). Pharmaceutical compositions are preferably manufactured under GMP conditions.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

The precise dosage administered of each active ingredient will vary depending upon any number of factors, including but not limited to, the type of animal and type of disease state being treated, the age of the animal, and the route(s) of administration.

The following non-limiting Preparations and Examples illustrate the preparation of compounds and salts of the present invention.

### General Experimental

The Preparations and Examples that follow illustrate the invention but do not limit the invention in any way. All starting materials are available commercially or described in the literature. All temperature are in ° C. Flash column chromatography was carried out using Merck silica gel 60 (9385) or Redisep silica. NMR was carried out using a Varian Mercury 400 MHz NMR spectrometer or a Jeol ECX 400 MHz NMR. Where it is stated that compounds were prepared in the manner described for an earlier Preparation or Example, the skilled person will appreciate that reaction times, number of equivalents of reagents and reaction temperatures may have been modified for each specific reaction, and that it may nevertheless be necessary, or desirable, to employ different work-up or purification conditions.

Where singleton compounds have been analysed by LCMS, there are several methods used. These are illustrated below.

The invention is illustrated by the following non-limiting Examples in which the following abbreviations and definitions are used:

AcOH—acetic acid; APCI—atmospheric pressure chemical ionization; Arbocel is a filter agent; br s—broad singlet; BINAP—2,2′-bis(diphenylphosphino)-1,1′-binapthyl; nBuLi—n-Butyllithium; CDCl₃—deuterated chloroform; Cs₂CO₃ is caesium carbonate; CuI is copper (I) iodide; Cu(OAc)₂ is copper (II) acetate; δ—chemical shift; d—doublet; DAD—diode array detector; DCE—1,2-dichloroethane

DCM—dichloromethane; DEA—diethylamine; DIBAL—Diisobutylaluminium hydride; DIPEA—diisopropylethylamine; DMAP—4-dimethylaminopyridine; DME—dimethoxyethane; DMF—N,N-dimethylformamide; DMF-DMA—N,N-dimethylformamide-dimethylacetal; DMSO—dimethylsulphoxide

DPPF—1,1′-bis(diphenylphosphino)ferrocene; ELSD—evaporative light scattering detector; ESI—electrospray ionization; Et₂O—diethylether; EtOAc/EA—ethyl acetate; EtOH—ethanol; g—gram; HATU—2-(7-azabenzotriazol-1-yl)-1,1,3,3-tetramethyluronium hexafluorophosphate; HBTU is O-benzotriazol-1-yl-N,N,N′,N′-tetramethyluronium hexafluorophosphate; HCl is hydrochloric acid; HOBT is N-hydroxybenzotriazole hydrate; HPLC—high pressure liquid chromatography; IPA—isopropyl alcohol; K₂CO₃ is potassium carbonate; KHSO₄ is potassium hydrogen sulphate; KOAc is potassium acetate; KOH is potassium hydroxide; K₃PO₄ is potassium phosphate tribasic; KF—potassium fluoride; L is litre; LCMS—liquid chromatography mass spectrometry; LiHMDS—Lithium hexamethyldisilazide; m—multiplet; mg—milligram; mL—millilitre; M/Z—Mass Spectrum Peak; MeCN—acetonitrile; MeOH—methanol; 2-MeTHF—2-methyltetrahydrofuran; MgSO₄ is magnesium sulphate; MnO₂-manganese dioxide; NaClO₂—sodium chlorite; NaH—sodium hydride; NaHCO₃—sodium hydrogencarbonate; Na₂CO₃—sodium carbonate; NaH₂PO₄—sodium phosphate; NaHSO₃—sodium bisulphite; NaHSO₄— sodium hydrogensulphate; NaOH—sodium hydroxide; Na₂SO₄—sodium sulphate; NH₃—ammonia; NH₄Cl—ammonium chloride; NMM—N-MethylMorpholine; NMR—nuclear magnetic resonance; Pd/C—palladium on carbon; PdCl₂—palladium dichloride; Pd₂(dba)₃ is tris(dibenzylideneacetone)dipalladium(0); Pd(PPh₃)₄—palladium tetrakis(triphenylphosphine); Pd(OAc)₂—palladium acetate; PTSA—para-toluenesulfonic acid; Prep—preparation; Rₜ—retention time; q—quartet; s—singlet; TBDMS—tertbutyldimethylsilyl; TBME—tertbutyldimethylether; TCP—1-propylphosphonic acid cyclic anhydride; TEA—triethylamine; TFA—trifluoroacetic acid; THF—tetrahydrofuran; TLC—thin layer chromatography; (R, S)—racemic mixture; WSCDI—1-(3-dimethylaminopropyl)-3-ethylcarbodiimide hydrochloride.

“CommAv” means a commercially available intermediate/reagent.

For the avoidance of doubt, named compounds used herein have been named using IUAPC, Chemdraw and/or Name Pro ACD Labs Name Software v7.11™ or using other standard nomenclature. NMR spectra were measured in deuterated solvents and were consistent with the names/structures given below.

Where singleton compounds have been purified by High Performance Liquid Chromatography, unless otherwise stated, one of four methods were used, and these are shown below.

Waters Purification Systems with mass spec or UV detection—AP3

### Prep System 1 (AP3 Acid)

10 minute prep LC-MS gradient and instrument conditions

A: 0.1% formic acid in water

B: 0.1% formic acid in acetonitrile

Column: C18 phase Sunfire 100×19.0 mm

Gradient: 95-2% A over 7 min, 2 min hold, 1 min re-equilibration, 18 mL/min flow rate

Temperature: ambient

### Prep System 2 (AP3 Base)

10 minute prep LC-MS gradient and instrument conditions

A: 0.1% DEA in water

B: 0.1% DEA in acetonitrile

Column: C18 phase Xterra 100×19.0 mm

Gradient: 95-2% A over 7 min, 2 min hold, 1 min re-equilibration, 18 mL/min flow rate

Temperature: ambient

### LCMS QC Conditions for Library Protocol 1/2

3 minute LC-MS gradient and instrument conditions

A: 0.05% formic acid in water

B: Acetonitrile

Column: RESTEK C18 30×2.1 mm 3 micron particle size

Gradient: Initial: 98% A; 2% B; 0.75 mins 98% A, 2% B; 1 min 90% A, 10% B; 2 mins 2% A, 98% B; 2.25 mins 2% A, 98% B; 2.90 mins 98% A, 2% B; 3 mins 98% a, 2% B.

Flow rate: 1.50 mL/min

UV: 215 nm—ELSD—MS

Temperature: 50° C.

### LCMS QC Conditions for Library Protocol 3

### Method A: AB01

Column: Welch XB-C18 2.1×50 mm 5 μm, 50° C., mobile phase A: 0.0375% TFA in water; mobile phase B: 0.01875% TFA in acetonitrile. Initial gradient 1% B; 0.60 mins 5% B, 4.00 mins 100% B, 4.30 mins 1% B, 4.70 mins 1% B. Flow rate 0.8 mL/min.

### Method B: AB10

Column: Welch XB-C18 2.1×50 mm 5 μm, 50° C., mobile phase A: 0.0375% TFA in water; mobile phase B: 0.01875% TFA in acetonitrile. Initial gradient 10% B; 0.60 mins 10% B, 4.00 mins 100% B, 4.30 mins 10% B, 4.70 mins 10% B. Flow rate 0.8 mL/min

The invention is illustrated by the following non-limiting examples in which the following abbreviations and definitions are used:

The Preparations and Examples that follow illustrate the invention but do not limit the invention in any way. All starting materials are available commercially or described in the literature. All temperature are in ° C. Flash column chromatography was carried out using Merck silica gel 60 (9385) or Redisep silica. NMR was carried out using a Varian Mercury 300/400 MHz NMR spectrometer or a Jeol ECX 400 MHz NMR.

The mass spectra were obtained using:

Waters ZQ ESCI

Applied Biosystem's API-2000 5 min LC-MS

Waters Alliance 2795 with ZQ2000 (ESI)

Aglient 110 HPLC 5 min (System 5)

Waters ZQ ESCI 8 min LC-MS

Waters Alliance 2695 with ZQ2000 (ESI) 25 min

HP 1100 HPLC with Waters Micromass ZQ mass detector 12.5 min LC-MS

UPLC mass spectra were obtained using a Waters Acquity ZQD (ESI) 1.5 min LC-MS WATERS ACQUITY UPLC/WATERS 3100 MSD/PL-ELS 2100 ICE ELSD

Where singleton compounds have been analysed by LCMS, there are six methods used. These are illustrated below.

System 1

6 minute LC-MS gradient and instrument conditions

A: 0.1% formic acid in water

B: 0.1% formic acid in acetonitrile

Column: C18 phase Waters Sunfire 50×4.6 mm with 5 micron particle size

Gradient: 95-5% A over 3 min, 1 min hold, 2 min re-equilibration, 1.5 mL/min flow rate

UV: 210 nm—450 nm DAD

Temperature: 50° C.

System 2

2 minute LC-MS gradient and instrument conditions

A: 0.1% formic acid in water

B: 0.1% formic acid in acetonitrile

Column: C18 phase Phenomenex 20×4.0 mm with 3 micron particle size

Gradient: 70-2% A over 1.5 min, 0.3 min hold, 0.2 re-equilbration, 1.8 mL/min flow rate

UV: 210 nm—450 nm DAD

Temperature: 75° C.

System 3

5 minute LC-MS gradient and instrument conditions

A: 0.1% formic acid in water

B: 0.1% formic acid in acetonitrile

Column: C18 phase Waters Sunfire 50×4.6 mm with 5 micron particle size

Gradient: 95-5% A over 3 min, 1 min hold, 1 min re-equilibration, 1.5 mL/min flow rate

UV: 225 nm—ELSD—MS

Temperature: ambient

System 4

5 minute LC-MS gradient and instrument conditions

A: 0.1% ammonium hydroxide in water

B: 0.1% ammonium hydroxide in acetonitrile

Column: C18 phase XTerra 50×4.6 mm with 5 micron particle size

Gradient: 95-5% A over 3 min, 1 min hold, 1 min re-equilibration, 1.5 mL/min flow rate

UV: 225 nm—ELSD—MS

Temperature: ambient

System 5

5 minute LC-MS gradient and instrument conditions

A: 0.0375% TFA in water

B: 0.01875% TFA in acetonitrile

Column: C18 phase Welch XB 50×2.1 mm with 5 micron particle size

Gradient: 99-0% A over 4 min, 0.70 min re-equilibration, 0.8 mL/min flow rate

UV: 225 nm—ELSD—MS

Temperature: 50° C.

System 9

5 minute LC-MS gradient and instrument conditions

A: 0.05% formic acid in water

B: acetonitrile

Column: C18 phase XBridge 50×4.6 mm with 5 micron particle size

Gradient: 90-10% A over 3 min, 1 min hold, 1 min re-equilibration, 1.2 mL/min flow rate

UV: 200 nm—260 nm DAD

Temperature: 25° C.

System 10

5 minute LC-MS gradient and instrument conditions

A: 10 mM ammonium acetate in water

B: acetonitrile

Column: C18 phase Gemini NX 50×4.6 mm with 5 micron particle size

Gradient: 90-10% A over 3 min, 1 min hold, 1 min re-equilibration, 1.2 mL/min flow rate

UV: 200 nm—260 nm DAD

Temperature: 25° C.

Where singleton compounds have been purified by High Performance Liquid Chromatography, unless otherwise stated, one of four methods were used, and these are shown below.

Waters Purification Systems with mass spec or UV detection

Prep System 1

10 minute prep LC-MS gradient and instrument conditions

A: 0.1% formic acid in water

B: 0.1% formic acid in acetonitrile

Column: C18 phase Sunfire 100×19.0 mm

Gradient: 95-2% A over 7 min, 2 min hold, 1 min re-equilibration, 18 mL/min flow rate

Temperature: ambient

Prep System 2

10 minute prep LC-MS gradient and instrument conditions

A: 0.1% DEA in water

B: 0.1% DEA in acetonitrile

Column: C18 phase Xterra 100×19.0 mm

Gradient: 95-2% A over 7 min, 2 min hold, 1 min re-equilibration, 18 mL/min flow rate

Temperature: ambient

Prep System 3

7 minute prep LC-MS gradient and instrument conditions

A: 0.05% ammonia in water

B: acetonitrile

Column: C18 phase Xbridge 50×19.0 mm

Gradient: 90-20% A over 7 min, 20 mL/min flow rate

Temperature: ambient

Prep System 4

8 minute prep LC-MS gradient and instrument conditions

A: 0.1% TFA in water

B: acetonitrile

Column: C18 phase Sepax BR 100×21.2 mm

Gradient: 96-33% A over 8 min, 30 mL/min flow rate

Temperature: ambient

Method: 1

Mobile phase:—A: 5 mM NH₄OAc in H₂O; B: Acetonitrile

Column name:—X Bridge Prep C₁₈ 5μ OBD (19×250 mm)

Gradient: 90-10% A over 16 min, 4 min hold, 3 min re-equilibration, 14.0 mL/min flow rate

Temperature: ambient

Waters auto purification instrument with PDA

Method: 2

Mobile phase:—A: 0.05% HCOOH in H₂O; B: Acetonitrile

Column name:—X terra Prep RP18 10μ (19×250 mm)

Gradient: 90-10% A over 16 min, 4 min hold, 3 min re-equilibration. 14.0 mL/min flow rate

Temperature: ambient

Waters auto purification instrument with PDA

Method: 3

Mobile phase:—A: 0.1% NH₃ in H₂O; B: Acetonitrile

Column name:—Gemini-NX 5μ C18 110A (100×30 mm)

Gradient: 90-10% A over 10 min, 2 min hold, 1 min re-equilibration. 30.0 mL/min flow rate

Temperature: ambient

Waters auto purification instrument with PDA

### Example 1

### 2-(4-chlorophenyl)-N-(4-{[7-(trans-4-hydroxycyclohexyl)-7H-pyrrolo[2,3-d]pyrimidin-5-yl]carbonyl}pyridin-2-yl)acetamide

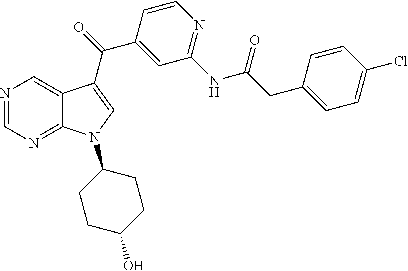 [SMILES: O=C(Cc1ccc(Cl)cc1)Nc1cc(C(=O)c2cn([C@H]3CC[C@H](O)CC3)c3ncncc23)ccn1]

Method 1—Step 1

A mixture of (2-aminopyridin-4-yl)[7-(trans-4-{[tert-butyl(dimethyl)silyl]oxy}cyclohexyl)-7H-pyrrolo[2,3-d]pyrimidin-5-yl]methanone (Preparation 7, 90 mg, 0.20 mmol), HATU (114 mg, 0.3 mmol) and 4-chlorophenylacetic acid (44 mg, 0.26 mmol) in pyridine (1 mL) was stirred at 50° C. for 18 hours. The reaction was cooled and partitioned between saturated aqueous NaHCO₃ solution (5 mL) and ethyl acetate (5 mL). The organic layer was collected, dried over MgSO₄ and concentrated in vacuo. The residue was purified using silica gel column chromatography eluting with a gradient of heptane:ethyl acetate 80:20 to 30:70 to afford the silyl protected amide precursor.

### Method 1—Step 2

The silyl protected amide precursor was dissolved in THF (3 mL) and 1N aqueous HCl (3 mL) was added to the solution. The reaction mixture was stirred at room temperature for 2 hours and 1 N NaOH (5 mL) solution was added to basify. The mixture was extracted with ethyl acetate three times (3×10 mL), the organic layer was collected, dried over MgSO₄ and concentrated in vacuo to obtain the crude residue. Ethyl acetate (5 mL) was added to the crude residue to precipitate a solid that was washed with additional ethyl acetate (5 mL) and dried in vacuo to give the desired compound as a white solid.

¹H NMR (400 MHz, DMSO-d₆): δ ppm 1.31-1.49 (m, 2H), 1.87-2.11 (m, 6H), 3.46-3.59 (m, 1H), 3.78 (s, 2H), 4.62-4.75 (m, 2H), 7.34-7.42 (m, 4H), 7.45 (dd, 1H), 8.35-8.39 (m, 1H), 8.42 (s, 1H), 8.54 (dd, 1H), 8.99 (s, 1H), 9.43 (s, 1H), 11.04 (s, 1H).

LCMS Rt=2.35 minutes MS m/z 490 [M³⁵Cl+H]⁺, 492 [M³⁷Cl+H]⁺

### Example 2

### 2-(4-cyanophenyl)-N-(4-{[7-(trans-4-hydroxycyclohexyl)-7H-pyrrolo[2,3-d]pyrimidin-5-yl]carbonyl}pyridine-2-yl)acetamide

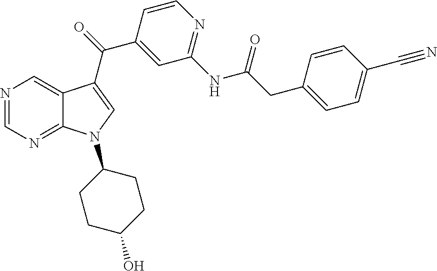 [SMILES: N#Cc1ccc(CC(=O)Nc2cc(C(=O)c3cn([C@H]4CC[C@H](O)CC4)c4ncncc34)ccn2)cc1]

Prepared according to Method 1 using 4-cyanophenylacetic acid.

LCMS Rt=2.08 minutes MS m/z 481 [M+H]⁺

### Example 3

### 2-(3,5-dimethyl-1H-pyrazol-1-yl)-N-(4-{[7-(trans-4-hydroxycyclohexyl)-7H-pyrrolo[2,3-d]pyrimidin-5-yl]carbonyl}pyridin-2-yl)acetamide

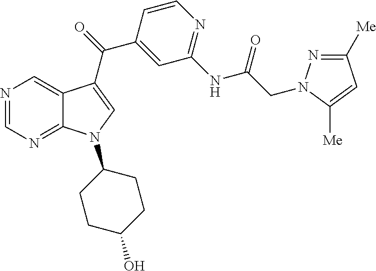 [SMILES: Cc1cc(C)n(CC(=O)Nc2cc(C(=O)c3cn([C@H]4CC[C@H](O)CC4)c4ncncc34)ccn2)n1]

Prepared according to Method 1 using (3,5-dimethyl-pyrazol-1-yl)acetic acid. After trituration the solid was dissolved in DMSO (1 mL) and purified using preparative HPLC.

LCMS Rt=2.57 minutes MS m/z 474 [M+H]⁺

### Example 4

### 2-(3,5-dimethyl-1H-pyrazol-1-yl)-N-(4-{[7-(cis-4-hydroxycyclohexyl)-7H-pyrrolo[2,3-d]pyrimidin-5-yl]carbonyl}pyridin-2-yl)acetamide

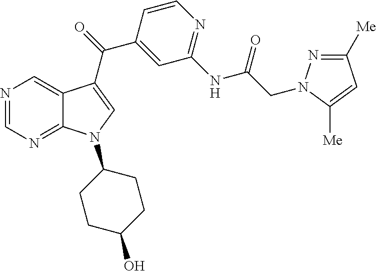 [SMILES: Cc1cc(C)n(CC(=O)Nc2cc(C(=O)c3cn([C@H]4CC[C@@H](O)CC4)c4ncncc34)ccn2)n1]

Prepared according to Method 1 using (3,5-dimethyl-pyrazol-1-yl)acetic acid and (2-aminopyridin-4-yl)[7-(cis-4-{[tert-butyl(dimethyl)silyl]oxy}cyclohexyl)-7H-pyrrolo[2,3-d]pyrimidin-5-yl]methanone (Preparation 9). After trituration the solid was dissolved in DMSO (1 mL) and purified using preparative HPLC.

LCMS Rt=2.65 minutes MS m/z 474 [M+H]⁺

### Example 5

### 2-(4-cyanophenyl)-N-(4-{[7-(cis-4-hydroxycyclohexyl)-7H-pyrrolo[2,3-d]pyrimidin-5-yl]carbonyl}pyridin-2-yl)acetamide

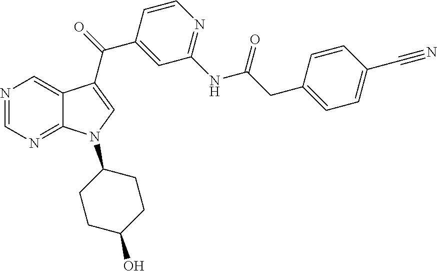 [SMILES: N#Cc1ccc(CC(=O)Nc2cc(C(=O)c3cn([C@H]4CC[C@@H](O)CC4)c4ncncc34)ccn2)cc1]

Prepared according to Method 1 using 4-cyanophenylacetic acid and (2-aminopyridin-4-yl)[7-(cis-4-{[tert-butyl(dimethyl)silyl]oxy}cyclohexyl)-7H-pyrrolo[2,3-d]pyrimidin-5-yl]methanone (Preparation 9).

LCMS Rt=2.17 minutes, MS m/z 481 [M+H]⁺

### Example 6

### 2-(4-chlorophenyl)-N-(4-{[7-(cis-4-hydroxycyclohexyl)-7H-pyrrolo[2,3-d]pyrimidin-5-yl]carbonyl}pyridin-2-yl)acetamide

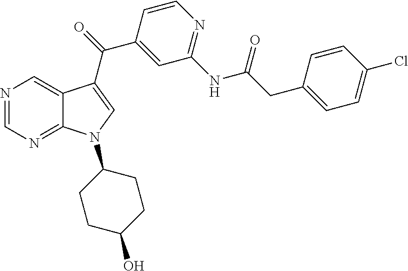 [SMILES: O=C(Cc1ccc(Cl)cc1)Nc1cc(C(=O)c2cn([C@H]3CC[C@@H](O)CC3)c3ncncc23)ccn1]

Prepared according to Method 1 using 4-chlorophenylacetic acid and (2-aminopyridin-4-yl)[7-(cis-4-{[tert-butyl(dimethyl)silyl]oxy}cyclohexyl)-7H-pyrrolo[2,3-d]pyrimidin-5-yl]methanone (Preparation 9).

LCMS Rt=2.46 minutes MS m/z 490 [M³⁵Cl+H]⁺, 492 M³⁷Cl+H]⁺

### Example 7

### N-{5-[2-Amino-7-(2-hydroxy-1,1-dimethyl-ethyl)-7H-pyrrolo[2,3-d]pyrimidine-5-carbonyl]-2-cyano-phenyl}-2-(4-trifluoromethyl-phenyl)-acetamide

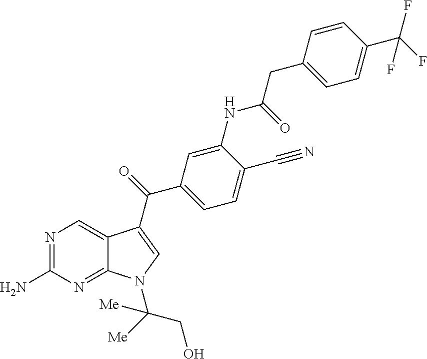 [SMILES: CC(C)(CO)n1cc(C(=O)c2ccc(C#N)c(NC(=O)Cc3ccc(C(F)(F)F)cc3)c2)c2cnc(N)nc21]

To a solution of N-(5-{2-amino-7-[2-(tert-butyl-dimethyl-silanyloxy)-1,1-dimethyl-ethyl]-7H-pyrrolo[2,3-d]pyrimidine-5-carbonyl}-2-cyano-phenyl)-2-(4-trifluoromethyl-phenyl)-acetamide (Preparation 1, 30 mg, 0.046 mmol) in THF (2 mL) was added TBAF (1 M solution in THF, 0.05 mL, 0.05 mmol) at 0° C. and the reaction was allowed to stir at room temperature for 2 hours. Additional TBAF (0.05 mL) was added and the reaction mixture was further stirred for 1 hour. The reaction was diluted with EtOAc and washed with saturated sodium bicarbonate solution, dried over sodium sulphate and evaporated in vacuo. The residue was purified by preparative TLC in 5% MeOH in DCM to afford the title compound as yellow solid in 49% yield, 12 mg.

¹H NMR (400 MHz, DMSO-d₆): δ ppm 1.62 (s, 6H), 3.88-3.91 (m, 4H), 4.98 (t, 1H), 6.53 (s, 2H), 7.60 (d, 2H), 7.66-7.71 (m, 4H), 7.98-8.00 (m, 2H), 8.95 (s, 1H), 10.68 (s, 1H).

LCMS: Rₜ=3.15 minutes MS m/z 537 [M+H]⁺

The following examples were prepared according to Method 2 (Example 36) followed by the method described for Example 7 using 2-Amino-4-{2-amino-7-[2-(tert-butyl-dimethyl-silanyloxy)-1,1-dimethyl-ethyl]-7H-pyrrolo[2,3-d]pyrimidine-5-carbonyl}-benzonitrile (Preparation 12) and the appropriate acetic acids. Purification was by preparative TLC eluting with 5% MeOH in DCM unless otherwise stated.

| Example | Name | Data |
|---|---|---|
| 8 | N-(5-{[2-amino-7-(1-hydroxy-2-methylpropan-2-yl)-7H- | MS m/z |
| | pyrrolo[2,3-d]pyrimidin-5-yl]carbonyl}-2-cyanophenyl)-2-(5- | 504 [M + H]⁺ |
| | chloropyridin-2-yl)acetamide | |
| 9 | N-(5-{[2-amino-7-(1-hydroxy-2-methylpropan-2-yl)-7H- | MS m/z |
| | pyrrolo[2,3-d]pyrimidin-5-yl]carbonyl}-2-cyanophenyl)-2-(4- | 503 [M + H]⁺ |
| | chlorophenyl)acetamide | |
| 10 | N-(5-{[2-amino-7-(1-hydroxy-2-methylpropan-2-yl)-7H- | MS m/z |
| | pyrrolo[2,3-d]pyrimidin-5-yl]carbonyl}-2-cyanophenyl)-2-[5- | 541 [M + H]⁺ |
| | methyl-3-(trifluoromethyl)-1H-pyrazol-1-yl]acetamide | |
| 11 | N-(5-{[2-amino-7-(1-hydroxy-2-methylpropan-2-yl)-7H- | MS m/z |
| | pyrrolo[2,3-d]pyrimidin-5-yl]carbonyl}-2-cyanophenyl)-2-[3- | 527 [M + H]⁺ |
| | (trifluoromethyl)-1H-pyrazol-1-yl]acetamide | |
| 12 | N-(5-{[2-amino-7-(1-hydroxy-2-methylpropan-2-yl)-7H- | MS m/z |
| | pyrrolo[2,3-d]pyrimidin-5-yl]carbonyl}-2-cyanophenyl)-2-[4- | 528 [M + H]⁺ |
| | (trifluoromethyl)-1H-1,2,3-triazol-1-yl]acetamide | |

The following examples were prepared according to Method 2 (Example 36) followed by method 1 Step 2 using 4M HCl in dioxan, using (4-aminopyridin-2-yl){7-[(1R)-1-methyl-2-(tetrahydro-2H-pyran-2-yloxy)ethyl]-7H-pyrrolo[2,3-d]pyrimidin-5-yl}methanone (Preparation 22) or (4-aminopyridin-2-yl){7-[(1S)-1-methyl-2-(tetrahydro-2H-pyran-2-yloxy)ethyl]-7H-pyrrolo[2,3-d]pyrimidin-5-yl}methanone (Preparation 23) and the appropriate acetic acids.

| Example | Name | Data |
|---|---|---|
| 13 | 2-(5-chloropyridin-2-yl)-N-[2-({7-[(2S)-1-hydroxypropan-2- | MS m/z |
| | yl]-7H-pyrrolo[2,3-d]pyrimidin-5-yl}carbonyl)pyridin-4- | 451 [M + H]⁺ |
| | yl]acetamide | |
| 14 | N-[2-({7-[(2S)-1-hydroxypropan-2-yl]-7H-pyrrolo[2,3- | MS m/z |
| | d]pyrimidin-5-yl}carbonyl)pyridin-4-yl]-2-[3-(trifluoromethyl)- | 474 [M + H]⁺ |
| | 1H-pyrazol-1-yl]acetamide | |
| 15 | 2-(4-cyanophenyl)-N-[2-({7-[(2S)-1-hydroxypropan-2-yl]-7H- | MS m/z |
| | pyrrolo[2,3-d]pyrimidin-5-yl}carbonyl)pyridin-4-yl]acetamide | 441 [M + H]⁺ |
| 16 | 2-(4-chlorophenyl)-N-[2-({7-[(2S)-1-hydroxypropan-2-yl]-7H- | MS m/z |
| | pyrrolo[2,3-d]pyrimidin-5-yl}carbonyl)pyridin-4-yl]acetamide | 450 [M + H]⁺ |
| 17 | N-[2-({7-[(2S)-1-hydroxypropan-2-yl]-7H-pyrrolo[2,3- | MS m/z |
| | d]pyrimidin-5-yl}carbonyl)pyridin-4-yl]-2-[4- | 484 [M + H]⁺ |
| | (trifluoromethyl)phenyl]acetamide | |
| 18 | 2-(5-fluoropyridin-2-yl)-N-[2-({7-[(2S)-1-hydroxypropan-2-yl]- | MS m/z |
| | 7H-pyrrolo[2,3-d]pyrimidin-5-yl}carbonyl)pyridin-4- | 435 [M + H]⁺ |
| | yl]acetamide | |
| 19 | N-[2-({7-[(2R)-1-hydroxypropan-2-yl]-7H-pyrrolo[2,3- | MS m/z |
| | d]pyrimidin-5-yl}carbonyl)pyridin-4-yl]-2-[3- | 484 [M + H]⁺ |
| | (trifluoromethyl)phenyl]acetamide | |
| 20 | N-[2-({7-[(2S)-1-hydroxypropan-2-yl]-7H-pyrrolo[2,3- | MS m/z |
| | d]pyrimidin-5-yl}carbonyl)pyridin-4-yl]-2-[5-methyl-3- | 488 [M + H]⁺ |
| | (trifluoromethyl)-1H-pyrazol-1-yl]acetamide | |
| 21 | N-[2-({7-[(2R)-1-hydroxypropan-2-yl]-7H-pyrrolo[2,3- | MS m/z |
| | d]pyrimidin-5-yl}carbonyl)pyridin-4-yl]-2-[5-methyl-3- | 488 [M + H]⁺ |
| | (trifluoromethyl)-1H-pyrazol-1-yl]acetamide | |
| 22 | N-[2-({7-[(2R)-1-hydroxypropan-2-yl]-7H-pyrrolo[2,3- | MS m/z |
| | d]pyrimidin-5-yl}carbonyl)pyridin-4-yl]-2-[4- | 484 [M + H]⁺ |
| | (trifluoromethyl)phenyl]acetamide | |
| 23 | 2-(4-cyanophenyl)-N-[2-({7-[(2R)-1-hydroxypropan-2-yl]-7H- | MS m/z |
| | pyrrolo[2,3-d]pyrimidin-5-yl}carbonyl)pyridin-4-yl]acetamide | 441 [M + H]⁺ |
| 24 | N-[2-({7-[(2S)-1-hydroxypropan-2-yl]-7H-pyrrolo[2,3- | MS m/z |
| | d]pyrimidin-5-yl}carbonyl)pyridin-4-yl]-2-[3- | 484 [M + H]⁺ |
| | (trifluoromethyl)phenyl]acetamide | |
| 25 | 2-(5-chloropyridin-2-yl)-N-[2-({7-[(2R)-1-hydroxypropan-2- | MS m/z |
| | yl]-7H-pyrrolo[2,3-d]pyrimidin-5-yl}carbonyl)pyridin-4- | 449 [M + H]⁻ |
| | yl]acetamide | |
| 26 | N-[2-({7-[(2R)-1-hydroxypropan-2-yl]-7H-pyrrolo[2,3- | MS m/z |
| | d]pyrimidin-5-yl}carbonyl)pyridin-4-yl]-2-[3-(trifluoromethyl)- | 474 [M + H]⁺ |
| | 1H-pyrazol-1-yl]acetamide | |
| 27 | 2-(5-fluoropyridin-2-yl)-N-[2-({7-[(2R)-1-hydroxypropan-2-yl]- | MS m/z |
| | 7H-pyrrolo[2,3-d]pyrimidin-5-yl}carbonyl)pyridin-4- | 435 [M + H]⁺ |
| | yl]acetamide | |
| 28 | 2-(4-chlorophenyl)-N-[2-({7-[(2R)-1-hydroxypropan-2-yl]-7H- | MS m/z |
| | pyrrolo[2,3-d]pyrimidin-5-yl}carbonyl)pyridin-4-yl]acetamide | 450 [M + H]⁺ |
| 29 | N-[2-({7-[(2S)-1-hydroxypropan-2-yl]-7H-pyrrolo[2,3- | MS m/z |
| | d]pyrimidin-5-yl}carbonyl)pyridin-4-yl]-2-[4-(propan-2-yl)-1H- | 449 [M + H]⁺ |
| | 1,2,3-triazol-1-yl]acetamide | |
| 30 | 2-(4-cyclopropyl-1H-1,2,3-triazol-1-yl)-N-[2-({7-[(2S)-1- | MS m/z |
| | hydroxypropan-2-yl]-7H-pyrrolo[2,3-d]pyrimidin-5- | 447 [M + H]⁺ |
| | yl}carbonyl)pyridin-4-yl]acetamide | |
| 31 | N-[2-({7-[(2S)-1-hydroxypropan-2-yl]-7H-pyrrolo[2,3- | MS m/z |
| | d]pyrimidin-5-yl}carbonyl)pyridin-4-yl]-2-[4-(trifluoromethyl)- | 475 [M + H]⁺ |
| | 1H-1,2,3-triazol-1-yl]acetamide | |

### Example 32

### 2-(4-cyanophenyl)-N-(4-{[7-(1-hydroxypropan-2-yl)-7H-pyrrolo[2,3-d]pyrimidin-5-yl]carbonyl}pyridin-2-yl)acetamide

The title compound was prepared according to Method 1 (Example 1, Step 1) using (Preparation 8) and 4-cyanophenylacetic acid. The residue was purified using silica gel column chromatography eluting with 50-100% EtOAc in heptane followed by 5% MeOH in EtOAc.

LCMS Rt=2.38 minutes MS m/z 441 [M+H]⁺

The racemic mixture was separated into the two enantiomers through a Chiralpak IA column, 20 minute run.

Example 33=Peak 2 Rt=14.64 minutes

Example 34=Peak 1 Rt=11.46 minutes

### Example 35

### N-(2-cyano-5-{[7-(propan-2-yl)-7H-pyrrolo[2,3-d]pyrimidin-5-yl]carbonyl}phenyl)-2-[5-methyl-3-(trifluoromethyl)-1H-pyrazol-1-yl]acetamide

The title compound was prepared according to Method 2 (Example 36) using 2-amino-4-[(7-isopropyl-7H-pyrrolo[2,3-d]pyrimidin-5-yl)carbonyl]benzonitrile (Preparation 11) and [5-methyl-3-(trifluoromethyl)-1H-pyrazol-1-yl]acetic acid with recrystallisation from DCM and heptanes.

LCMS Rt=3.21 minutes MS m/z 496 [M+H]⁺

### Example 36

### N-[2-(7-tert-Butyl-7H-pyrrolo[2,3-d]pyrimidine-5-carbonyl)-pyridin-4-yl]-2-(4-cyclopropyl-[1,2,3]triazol-1-yl)-acetamide

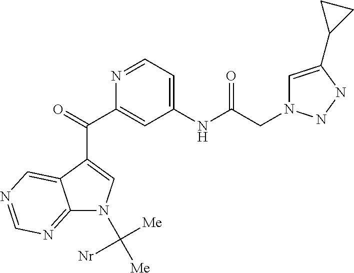

### Method 2

(4-Cyclopropyl-[1,2,3]triazol-1-yl)-acetic acid (Preparation 121, 85 mg, 0.508 mmol) and (4-Amino-pyridin-2-yl)-(7-tert-butyl-7H-pyrrolo[2,3-d]pyrimidin-5-yl)-methanone (Preparation 29, 100 mg, 0.338 mmol) were dissolved in THF (5 mL), followed by the addition of Et₃N (0.165 mL, 1.18 mmol) and 1-propylphosphonic acid cyclic anhydride (50% solution in EtOAc, 0.507 mL, 0.845 mmol) at room temperature. The resultant mixture was stirred at room temperature for 16 hours and then diluted with ethyl acetate (20 mL). The organic layer was washed with aqueous saturated NaHCO₃ solution, water, brine, dried over sodium sulphate and evaporated to dryness in vacuo. The crude residue was purified using preparative TLC eluting with 3% MeOH in DCM to afford the title compound as brown solid in 25% yield, 37 mg.

¹H NMR (400 MHz, DMSO-d₆): δ ppm 0.72-0.73 (m, 2H), 0.90-0.93 (m, 2H), 1.82 (s, 9H), 1.95-1.98 (m, 1H), 5.29 (s, 2H), 7.82 (d, 1H), 7.86 (s, 1H), 8.23 (s, 1H), 8.65 (d, 1H), 8.97 (s, 1H), 9.20 (s, 1H), 9.59 (s, 1H), 11.16 (br, 1H).

LCMS Rt=3.17 minutes MS m/z 445 [M+H]⁺

The following examples were prepared according to Method 2 (Example 36) at reflux using (4-amino-pyridin-2-yl)-(7-tert-butyl-7H-pyrrolo[2,3-d]pyrimidin-5-yl)-methanone (Preparation 29) and the appropriate acetic acids. Purification was by preparative HPLC unless otherwise stated.

| Example | Name/Structure | Data |
|---|---|---|
| 37 | N-{2-[(7-tert-butyl-7H-pyrrolo[2,3-d]pyrimidin-5- | MS mz/ |
| | yl)carbonyl]pyridin-4-yl}-2-(5-fluoropyridin-2-yl)acetamide | 433 [M + H]⁺ |
| 38 | N-{2-[(7-tert-butyl-7H-pyrrolo[2,3-d]pyrimidin-5- | MS mz/ |
| | yl)carbonyl]pyridin-4-yl}-2-[3- | 492 [M + H]⁺ |
| | (methylsulfonyl)phenyl]acetamide | |
| 39 | N-{2-[(7-tert-butyl-7H-pyrrolo[2,3-d]pyrimidin-5- | MS mz/ |
| | yl)carbonyl]pyridin-4-yl}-2-(5-chloropyridin-2-yl)acetamide | 449 [M + H]⁺ |
| 40 | N-{2-[(7-tert-butyl-7H-pyrrolo[2,3-d]pyrimidin-5- | MS mz/ |
| | yl)carbonyl]pyridin-4-yl}-2-(3-cyclopropyl-1H-pyrazol-1- | 444 [M + H]⁺ |
| | yl)acetamide | |
| 41 | N-{2-[(7-tert-butyl-7H-pyrrolo[2,3-d]pyrimidin-5- | MS mz/ |
| | yl)carbonyl]pyridin-4-yl}-2-[3-(trifluoromethyl)-1H-pyrazol-1- | 472 [M + H]⁺ |
| | yl]acetamide | |
| 42 | N-{2-[(7-tert-butyl-7H-pyrrolo[2,3-d]pyrimidin-5- | MS mz/ |
| | yl)carbonyl]pyridin-4-yl}-2-(4-chlorophenyl)acetamide | 448 [M + H]⁺ |
| 43 | N-{2-[(7-tert-butyl-7H-pyrrolo[2,3-d]pyrimidin-5- | MS mz/ |
| | yl)carbonyl]pyridin-4-yl}-2-(4-fluorophenyl)acetamide | 432 [M + H]⁺ |

### Example 44

### N-[4-(2-Amino-7-isopropyl-7H-pyrrolo[2,3-d]pyrimidine-5-carbonyl)-pyridin-2-yl]-2-(4-chloro-phenyl)-acetamide

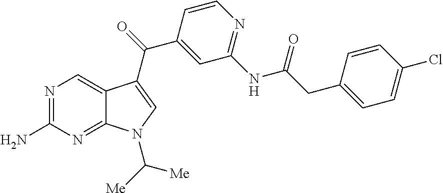 [SMILES: CC(C)n1cc(C(=O)c2ccnc(NC(=O)Cc3ccc(Cl)cc3)c2)c2cnc(N)nc21]

(4-Chloro-phenyl)-acetic acid (14 mg, 0.082 mmol) and (2-amino-7-isopropyl-7H-pyrrolo[2,3-d]pyrimidin-5-yl)(2-aminopyridin-4-yl)methanone (Preparation 31, 25 mg, 0.082 mmol) were dissolved in THF (2 mL) followed by the addition of Et₃N (0.041 mL, 0.294 mmol) and T₃P (50% solution in EtOAc, 0.15 mL, 0.252 mmol) at room temperature. The resultant mixture was stirred at room temperature for 16 hours and then diluted with ethyl acetate. The organic layer was washed with aqueous NaHCO₃, water, brine, dried over sodium sulphate and evaporated to dryness in vacuo. The residue was purified using preparative TLC eluting with 3% MeOH in DCM to afford the title compound as pale yellow solid in 28% yield, 10 mg.

¹H NMR (400 MHz, DMSO-d₆): δ ppm 1.43 (d, 6H), 3.76 (s, 2H), 4.80-4.82 (m, 1H), 6.63 (s, 2H), 7.37 (s, 5H), 7.90 (s, 1H), 8.32 (s, 1H), 8.51 (d, 1H), 8.91 (s, 1H), 11.07 (s, 1H).

LCMS Rt=2.98 minutes MS m/z 449 [M+H]⁺

### Example 45

### N-(4-{[2-amino-7-(propan-2-yl)-7H-pyrrolo[2,3-d]pyrimidin-5-yl]carbonyl}pyridin-2-yl)-2-(4-cyanophenyl)acetamide

Prepared according to Example 44 using 4-cyanophenylacetic acid.

LCMS (System 9) Rt=2.75 minutes MS m/z 440 [M+H]⁺

### Example 46

### N-(4-{[2-amino-7-(propan-2-yl)-7H-pyrrolo[2,3-d]pyrimidin-5-yl]carbonyl}pyridin-2-yl)-2-[5-methyl-3-(trifluoromethyl)-1H-pyrazol-1-yl]acetamide

Prepared according to Example 44 using 5-methyl-3-(trifluoromethyl)-1H-pyrazol-1-yl]acetic acid.

LCMS (System 9): Rt=2.79 minutes MS m/z 487 [M+H]⁺

### Example 47

### 2-(4-Chloro-phenyl)-N-[2-(7-isopropyl-7H-pyrrolo[2,3-d]pyrimidine-5-carbonyl)-pyridin-4-yl]-acetamide

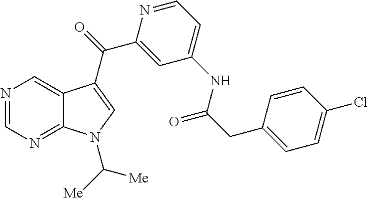 [SMILES: CC(C)n1cc(C(=O)c2cc(NC(=O)Cc3ccc(Cl)cc3)ccn2)c2cncnc21]

(4-Chloro-phenyl)-acetic acid (54.63 mg, 0.32 mmol) and (4-aminopyridin-2-yl)(7-isopropyl-7H-pyrrolo[2,3-d]pyrimidin-5-yl)methanone (Preparation 17, 90 mg, 0.32 mmol) were dissolved in THF (7 mL) followed by the addition of Et₃N (0.16 mL, 1.12 mmol) and T₃P (50% solution in EtOAc, 0.48 mL, 0.80 mmol) at room temperature. The resultant mixture was stirred at room temperature for 16 hours and then diluted with ethyl acetate. The organic layer was washed with aqueous NaHCO₃, water, brine, dried over sodium sulphate and evaporated to dryness in vacuo. The residue was purified using preparative TLC eluting with 3% MeOH in DCM to afford the title compound as pale yellow solid in 22% yield, 30 mg.

¹H NMR (400 MHz, DMSO-d₆): δ ppm 1.58 (d, 6H), 3.76 (s, 2H), 5.06-5.11 (m, 1H), 7.36-7.42 (m, 4H), 7.88 (d, 1H), 8.27 (s, 1H), 8.67 (d, 1H), 8.96 (s, 1H), 9.18 (s, 1H), 9.55 (s, 1H), 10.82 (s, 1H). LCMS Rt=3.27 minutes MS m/z 434 [M+H]⁺

### Example 48

### N-{2-[2-Amino-7-(2-hydroxy-1,1-dimethyl-ethyl)-7H-pyrrolo[2,3-d]pyrimidine-5-carbonyl}-pyridin-4-yl]-2-(4-chloro-phenyl)-acetamide

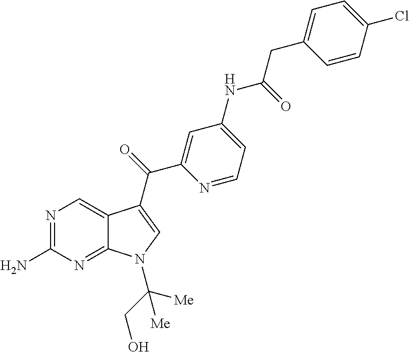 [SMILES: CC(C)(CO)n1cc(C(=O)c2cc(NC(=O)Cc3ccc(Cl)cc3)ccn2)c2cnc(N)nc21]

{2-Amino-7-[1,1-dimethyl-2-(tetrahydro-2H-pyran-2-yloxy)ethyl]-7H-pyrrolo[2,3-d]pyrimidin-5-yl}(4-aminopyridin-2-yl)methanone (Preparation 16, 70 mg, 0.17 mmol) and (4-chloro-phenyl)-acetic acid (29 mg, 0.17 mmol) were dissolved in THF (2 mL) followed by the addition of Et₃N (0.084 mL, 0.60 mmol) and 1-propylphosphonic acid cyclic anhydride (50% solution in EtOAc, 0.30 mL, 0.51 mmol). The mixture was stirred at room temperature for 16 hours and then evaporated in vacuo. The residue was dissolved in 10% HCl in dioxane (10 mL/mmol) and stirred at room temperature for 4 hours. The mixture was evaporated to dryness in vacuo, basified with saturated NaHCO₃ solution and extracted with 10% IPA-DCM solution (3 times). The combined organic layers were then washed with brine, dried over sodium sulphate and evaporated in vacuo. The residue was purified using preparative TLC eluting with 6% MeOH in DCM to afford the title compound as pale yellow solid in 33% yield, 27 mg.

¹H NMR (400 MHz, DMSO-d₆): δ ppm 1.66 (s, 6H), 3.75 (s, 2H), 3.92 (d, 2H), 5.03 (t, 1H), 6.48 (s, 2H), 7.35-7.41 (dd, 4H), 7.84 (dd, 1H), 8.18 (s, 1H), 8.60 (d, 1H), 8.73 (s, 1H), 9.06 (s, 1H), 10.80 (s, 1H).

LCMS Rt=3.08 minutes MS m/z 479 [M+H]⁺

The following examples were prepared according to the method described for Example 48 using {2-amino-7-[1,1-dimethyl-2-(tetrahydro-2H-pyran-2-yloxy)ethyl]-7H-pyrrolo[2,3-d]pyrimidin-5-yl}(4-aminopyridin-2-yl)methanone (Preparation 16) and the appropriate acetic acids.

| Example | Name | Data |
|---|---|---|
| 49 | N-(2-{[2-amino-7-(1-hydroxy-2-methylpropan-2-yl)-7H- | MS m/z 513 |
| | pyrrolo[2,3-d]pyrimidin-5-yl]carbonyl}pyridin-4-yl)-2-[3- | [M + H]⁺ |
| | (trifluoromethyl)phenyl]acetamide | |
| 50 | N-(2-{[2-amino-7-(1-hydroxy-2-methylpropan-2-yl)-7H- | MS m/z 513 |
| | pyrrolo[2,3-d]pyrimidin-5-yl]carbonyl}pyridin-4-yl)-2-[4- | [M + H]⁺ |
| | (trifluoromethyl)phenyl]acetamide | |
| 51 | N-(2-{[2-amino-7-(1-hydroxy-2-methylpropan-2-yl)-7H- | MS m/z 470 |
| | pyrrolo[2,3-d]pyrimidin-5-yl]carbonyl}pyridin-4-yl)-2-(4- | [M + H]⁺ |
| | cyanophenyl)acetamide | |
| 52 | N-(2-{[2-amino-7-(1-hydroxy-2-methylpropan-2-yl)-7H- | MS m/z 480 |
| | pyrrolo[2,3-d]pyrimidin-5-yl]carbonyl}pyridin-4-yl)-2-(5- | [M + H]⁺ |
| | chloropyridin-2-yl)acetamide | |
| 53 | N-(2-{[2-amino-7-(1-hydroxy-2-methylpropan-2-yl)-7H- | MS m/z 504 |
| | pyrrolo[2,3-d]pyrimidin-5-yl]carbonyl}pyridin-4-yl)-2-[4- | [M + H]⁺ |
| | (trifluoromethyl)-1H-1,2,3-triazol-1-yl]acetamide | |

The following examples were prepared according to the method described for Example 48 using (4-Aminopyridin-2-yl)[7-(2-{[tert-butyl(dimethyl)silyl]oxy}-1,1-dimethylethyl)-7H-pyrrolo[2,3-d]pyrimidin-5-yl]methanone (Preparation 35) and the appropriate acetic acids.

| Example | Name | Data |
|---|---|---|
| 54 | N-(2-{[7-(1-hydroxy-2-methylpropan-2-yl)-7H-pyrrolo[2,3- | MS m/z 489 |
| | d]pyrimidin-5-yl]carbonyl}pyridin-4-yl)-2-[4- | [M + H]⁺ |
| | (trifluoromethyl)-1H-1,2,3-triazol-1-yl]acetamide | |
| 55 | N-(2-{[7-(1-hydroxy-2-methylpropan-2-yl)-7H-pyrrolo[2,3- | MS m/z 498 |
| | d]pyrimidin-5-yl]carbonyl}pyridin-4-yl)-2-[4- | [M + H]⁺ |
| | (trifluoromethyl)phenyl]acetamide | |
| 56 | N-(2-{[7-(1-hydroxy-2-methylpropan-2-yl)-7H-pyrrolo[2,3- | m/z 498 |
| | d]pyrimidin-5-yl]carbonyl}pyridin-4-yl)-2-[3- | [M + H]⁺ |
| | (trifluoromethyl)phenyl]acetamide | |
| 57 | 2-(4-cyanophenyl)-N-(2-{[7-(1-hydroxy-2-methylpropan-2- | MS m/z 455 |
| | yl)-7H-pyrrolo[2,3-d]pyrimidin-5-yl]carbonyl}pyridin-4- | [M + H]⁺ |
| | yl)acetamide | |
| 58 | 2-(5-chloropyridin-2-yl)-N-(2-{[7-(1-hydroxy-2- | MS m/z 465 |
| | methylpropan-2-yl)-7H-pyrrolo[2,3-d]pyrimidin-5- | [M + H]⁺ |
| | yl]carbonyl}pyridin-4-yl)acetamide | |
| 59 | N-(2-{[7-(1-hydroxy-2-methylpropan-2-yl)-7H-pyrrolo[2,3- | MS m/z 488 |
| | d]pyrimidin-5-yl]carbonyl}pyridin-4-yl)-2-[3- | [M + H]⁺ |
| | (trifluoromethyl)-1H-pyrazol-1-yl]acetamide | |
| 60 | N-(2-{[7-(1-hydroxy-2-methylpropan-2-yl)-7H-pyrrolo[2,3- | MS m/z 502 |
| | d]pyrimidin-5-yl]carbonyl}pyridin-4-yl)-2-[5-methyl-3- | [M + H]⁺ |
| | (trifluoromethyl)-1H-pyrazol-1-yl]acetamide | |
| 61 | 2-(3-cyclopropyl-1H-pyrazol-1-yl)-N-(2-{[7-(1-hydroxy-2- | MS m/z 460 |
| | methylpropan-2-yl)-7H-pyrrolo[2,3-d]pyrimidin-5- | [M + H]⁺ |
| | yl]carbonyl}pyridin-4-yl)acetamide | |

### Example 62

### 2-(5-Cyanopyridin-2-yl)-N-{4-[(7-isopropyl-7H-pyrrolo[2,3-d]pyrimidin-5-yl)carbonyl}pyridin-2-yl]acetamide

Zinc cyanide (18.3 mg, 0.15 mmol) was added to 2-(5-bromopyridin-2-yl)-N-{4-[(7-isopropyl-7H-pyrrolo[2,3-d]pyrimidin-5-yl)carbonyl]pyridin-2-yl}acetamide (Preparation 181, 50 mg, 0.10 mmol) in DMF (1.5 mL) and the mixture was degassed with argon for 10 minutes. Then tris(dibenzylideneacetone)dipalladium (4 mg, 0.002 mmol) and 1,1′-bis(diphenylphosphino)ferrocene (5 mg, 0.008 mmol) were added and the mixture was heated at 100° C. for 30 minutes under microwave irradiation. The reaction was diluted with EtOAc (3 mL) and washed with water (2 mL), brine (2 mL) and dried over sodium sulphate. The filtrate was evaporated in vacuo and purified by preparative TLC eluting with 5% MeOH in DCM to afford the title compound was a white solid in 47% yield, 20 mg.

LCMS (System 9): Rt=2.85 minutes MS m/z 426 [M+H]⁺

### Example 63

### N-(2-{[2-amino-7-(propan-2-yl)-7H-pyrrolo[2,3-d]pyrimidin-5-yl]carbonyl}pyridin-4-yl)-2-(5-cyanopyridin-2-yl)acetamide

The title compound was prepared according to Example 62 using N-(2-{[2-amino-7-(propan-2-yl)-7H-pyrrolo[2,3-d]pyrimidin-5-yl]carbonyl}pyridin-4-yl)-2-(5-bromopyridin-2-yl)acetamide (Example 64).

LCMS (System 10): Rt=2.69 minutes MS m/z 441 [M+H]⁺

### Example 64

### N-(2-{[2-amino-7-(propan-2-yl)-7H-pyrrolo[2,3-d]pyrimidin-5-yl]carbonyl}pyridin-4-yl)-2-(5-bromopyridin-2-yl)acetamide

The title compound was prepared according to Method 3 (Example 66) using (2-amino-7-isopropyl-7H-pyrrolo[2,3-d]pyrimidin-5-yl)(4-aminopyridin-2-yl)methanone (Preparation 20) and (5-bromopyridin-2-yl)acetic acid (Preparation 133).

LCMS (System 10): Rt=3.15 min MS m/z 494 [M+H]⁺

### Example 65

### 2-(4-chlorophenyl)-N-(6-{[7-(propan-2-yl)-7H-pyrrolo[2,3-d]pyrimidin-5-yl]carbonyl}pyrimidin-4-yl)acetamide

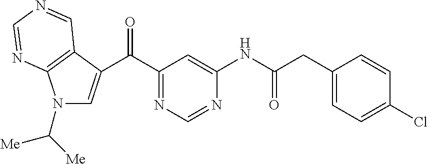 [SMILES: CC(C)n1cc(C(=O)c2cc(NC(=O)Cc3ccc(Cl)cc3)ncn2)c2cncnc21]

To (6-aminopyrimidin-4-yl)(7-isopropyl-7H-pyrrolo[2,3-d]pyrimidin-5-yl)methanone (Preparation 30, 40 mg, 0.14 mmol) in THF (3 mL) was added NaHMDS (0.592 mL, 0.6M, 0.355 mmol) at 0° C. and the reaction stirred for 10 minutes. (4-chlorophenyl)acetyl chloride (40.3 mg, 0.23 mmol) was added at 0° C. and the reaction stirred for 2 hours. A further 1.5 equivalents of (4-chlorophenyl)acetyl chloride (40.43 mg, 0.213 mmol) were added and the reaction continued stirring at room temperature for 16 hours before quenching with water. The reaction was extracted into DCM:MeOH 95:5 three times, the organic layers were combined, washed with brine and concentrated in vacuo. The residue was dissolved in DMSO (1 mL), filtered and purified using preparative HPLC to afford the title compound.

LCMS Rt=3.27 minutes MS m/z 435 [M+H]⁺

### Example 66

### 2-(3,4-Dichlorophenyl)-N-{4-[(7-isopropyl-7H-pyrrolo[2,3-d]pyrimidin-5-yl)carbonyl]pyridin-2-yl}acetamide

### Method 3

3,4-Dichlorophenylacetic acid (130 μmol) was added to (2-aminopyridin-4-yl)(7-isopropyl-7H-pyrrolo[2,3-d]pyrimidin-5-yl)methanone (Preparation 24, 500 μL, 0.2 M in THF) then 1-propylphosphonic acid cyclic anhydride (3 eq) and DIPEA (3.5 eq) was added. The mixture was stirred at 25° C. for 16 hours and then evaporated in vacuo and purified by preparative HPLC to afford the title compound in 15% yield, 10 mg.

LCMS (system 8): Rt=1.81 min MS m/z 468 [M+H]⁺

The following examples were prepared according to Methods 1, 2 or 3 using (2-aminopyridin-4-yl)(7-isopropyl-7H-pyrrolo[2,3-d]pyrimidin-5-yl)methanone (Preparation 24) and the appropriate acetic acids.

| Example | Name | Data |
|---|---|---|
| 67 | 2-(4-chlorophenyl)-N-(4-{[7- | MS m/z 434, 436 [M + H]⁺ |
| | (propan-2-yl)-7H-pyrrolo[2,3- | |
| | d]pyrimidin-5- | |
| | yl]carbonyl}pyridin-2- | |
| | yl)acetamide | |
| 68 | 2-(4-cyanophenyl)-N-(4-{[7- | MS m/z 425 [M + H]⁺ |
| | (propan-2-yl)-7H-pyrrolo[2,3- | |
| | d]pyrimidin-5- | |
| | yl]carbonyl}pyridin-2- | |
| | yl)acetamide | |
| 69 | 2-[5-methyl-3-(trifluoromethyl)- | MS m/z 472 [M + H]⁺ |
| | 1H-pyrazol-1-yl]-N-(2-{[7- | |
| | (propan-2-yl)-7H-pyrrolo[2,3- | |
| | d]pyrimidin-5- | |
| | yl]carbonyl}pyridin-4- | |
| | yl)acetamide | |
| 70 | 2-(3-methyl-1H-pyrazol-1-yl)- | MS m/z 404 [M + H]⁺ |
| | N-(4-{[7-(propan-2-yl)-7H- | |
| | pyrrolo[2,3-d]pyrimidin-5- | |
| | yl]carbonyl}pyridin-2- | |
| | yl)acetamide | |
| 71 | 2-[4-cyano-3- | ¹H NMR (400 MHz, DMSO-d₆): δ ppm |
| | (trifluoromethyl)phenyl]-N-(4- | 1.50 (d, 6H), 4.04 (s, 2H), 5.08 (m, 1H), |
| | {[7-(propan-2-yl)-7H- | 7.48 (d, 1H), 7.85 (d, 1H), 8.02 (s, 1H), |
| | pyrrolo[2,3-d]pyrimidin-5- | 8.14 (d, 1H), 8.38 (s, 1H), 8.45 (s, 1H), |
| | yl]carbonyl}pyridin-2- | 8.56 (d, 1H), 8.99 (s, 1H), 9.42 (s, 1H), |
| | yl)acetamide | 11.15 (s, 1H). |
| 72 | 2-[4-(cyclopropyloxy)phenyl]- | MS m/z 456 [M + H]⁺ |
| | N-(4-{[7-(propan-2-yl)-7H- | |
| | pyrrolo[2,3-d]pyrimidin-5- | |
| | yl]carbonyl}pyridin-2- | |
| | yl)acetamide | |
| 73 | 2-(5-fluoropyridin-2-yl)-N-(4- | ¹H NMR (400 MHz, DMSO-d₆): δ ppm |
| | {[7-(propan-2-yl)-7H- | 1.52 (d, 6H), 4.00 (s, 2H), 5.07 (m, 1H), |
| | pyrrolo[2,3-d]pyrimidin-5- | 7.47-7.50 (m, 2H), 7.70 (m, 1H), 8.39 (s, |
| | yl]carbonyl}pyridin-2- | 1H), 8.45 (s, 1H), 8.49 (d, 1H), 8.55 (d, |
| | yl)acetamide | 1H), 8.99 (s, 1H), 9.43 (s, 1H), 11.05 (s, |
| | | 1H). |
| 74 | 2-(5-chloropyridin-2-yl)-N-(4- | ¹H NMR (400 MHz, DMSO-d₆): δ ppm |
| | {[7-(propan-2-yl)-7H- | 1.53 (d, 6H), 4.00 (s, 2H), 5.07 (m, 1H), |
| | pyrrolo[2,3-d]pyrimidin-5- | 7.45 (s, 1H), 7.46 (m, 1H), 7.90 (dd, |
| | yl]carbonyl}pyridin-2- | 1H), 8.39 (s, 1H), 8.45 (s, 1H), |
| | yl)acetamide | 8.54-8.57 (m, 2H), 8.99 (s, 1H), 9.43 (s, 1H), |
| | | 11.07 (s, 1H). |

The following examples were prepared according to Methods 1, 2 or 3 using (2-amino-6-methylpyridin-4-yl)(7-isopropyl-7H-pyrrolo[2,3-d]pyrimidin-5-yl)methanone (Preparation 25) and the appropriate acetic acids, and purified using preparative HPLC.

| Example | Name | Data |
|---|---|---|
| 75 | N-(6-methyl-4-{[7-(propan-2-yl)-7H-pyrrolo[2,3-d]pyrimidin- | MS m/z 414 |
| | 5-yl]carbonyl}pyridin-2-yl)-2-phenylacetamide | [M + H]⁺ |
| 76 | 2-(4-cyanophenyl)-N-(6-methyl-4-{[7-(propan-2-yl)-7H- | MS m/z 439 |
| | pyrrolo[2,3-d]pyrimidin-5-yl]carbonyl}pyridin-2- | [M + H]⁺ |
| | yl)acetamide | |
| 77 | 2-(4-fluorophenyl)-N-(6-methyl-4-{[7-(propan-2-yl)-7H- | MS m/z 432 |
| | pyrrolo[2,3-d]pyrimidin-5-yl]carbonyl}pyridin-2- | [M + H]⁺ |
| | yl)acetamide | |
| 78 | 2-[4-cyano-3-(trifluoromethyl)phenyl]-N-(6-methyl-4-{[7- | MS m/z 507 |
| | (propan-2-yl)-7H-pyrrolo[2,3-d]pyrimidin-5- | [M + H]⁺ |
| | yl]carbonyl}pyridin-2-yl)acetamide | |

The following examples were prepared according to Methods 1, 2 or 3 using (4-Aminopyridin-2-yl)(7-isopropyl-7H-pyrrolo[2,3-d]pyrimidin-5-yl)methanone (Preparation 17) and the appropriate acetic acids, and purified using preparative HPLC.

| Example | Name | Data |
|---|---|---|
| 79 | N-(2-{[7-(propan-2-yl)-7H-pyrrolo[2,3- | MS m/z 459 [M + H]⁺ |
| | d]pyrimidin-5-yl]carbonyl}pyridin-4-yl)-2-[4- | |
| | (trifluoromethyl)-1H-1,2,3-triazol-1- | |
| | yl]acetamide | |
| 80 | N-(2-{[7-(propan-2-yl)-7H-pyrrolo[2,3- | MS m/z 425 [M + H]⁺ |
| | d]pyrimidin-5-yl]carbonyl}pyridin-4-yl)-2-[4- | |
| | (trifluoromethyl)-1H-1,2,3-triazol-1- | |
| | yl]acetamide | |
| 81 | 2-[3-(methylsulfonyl)phenyl]-N-(2-{[7- | MS m/z 478 [M + H]⁺ |
| | (propan-2-yl)-7H-pyrrolo[2,3-d]pyrimidin-5- | |
| | yl]carbonyl}pyridin-4-yl)acetamide | |
| 82 | 2-(4-cyclopropyl-1H-1,2,3-triazol-1-yl)-N- | MS m/z 431 [M + H]⁺ |
| | (2-{[7-(propan-2-yl)-7H-pyrrolo[2,3- | |
| | d]pyrimidin-5-yl]carbonyl}pyridin-4- | |
| | yl)acetamide | |
| 83 | 2-(3-cyclopropyl-1H-pyrazol-1-yl)-N-(2-{[7- | ¹H NMR (400 MHz, DMSO- |
| | (propan-2-yl)-7H-pyrrolo[2,3-d]pyrimidin-5- | d₆): δ ppm 0.60 (d, 2H), |
| | yl]carbonyl}pyridin-4-yl)acetamide | 0.82 (d, 2H), 1.54 (d, 6H), |
| | | 1.85 (m, 1H), 4.89 (m, 1H), |
| | | 4.96 (s, 1H), 5.96 (s, 1H), 7.60 (s, |
| | | 1H), 7.73-7.75 (d, 1H), |
| | | 7.83 (d, 1H), 8.21 (s, 1H), 8.38 (d, |
| | | 1H), 8.66 (d, 1H), 9.05 (s, |
| | | 1H), 9.54 (s, 1H), 10.90 (s, |
| | | 1H). |

The following examples were prepared according to Method 3 using (2-amino-7-isopropyl-7H-pyrrolo[2,3-d]pyrimidin-5-yl)(4-aminopyridin-2-yl)methanone (Preparation 20) and the appropriate acetic acids.

| Example | Name | Data |
|---|---|---|
| 84 | N-(2-{[2-amino-7-(propan-2-yl)-7H-pyrrolo[2,3- | MS m/z 487 |
| | d]pyrimidin-5-yl]carbonyl}pyridin-4-yl)-2-[5-methyl-3- | [M + H]⁺ |
| | (trifluoromethyl)-1H-pyrazol-1-yl]acetamide | |
| 85 | N-(2-{[2-amino-7-(propan-2-yl)-7H-pyrrolo[2,3- | MS m/z 473 |
| | d]pyrimidin-5-yl]carbonyl}pyridin-4-yl)-2-[3- | [M + H]⁺ |
| | (trifluoromethyl)-1H-pyrazol-1-yl]acetamide | |
| 86 | N-(2-{[2-amino-7-(propan-2-yl)-7H-pyrrolo[2,3- | MS m/z 450 |
| | d]pyrimidin-5-yl]carbonyl}pyridin-4-yl)-2-5- | [M + H]⁺ |
| | chloropyridin-2-yl)acetamide | |
| 87 | N-(2-{[2-amino-7-(propan-2-yl)-7H-pyrrolo[2,3- | ¹H NMR (400 MHz, |
| | d]pyrimidin-5-yl]carbonyl}pyridin-4-yl)-2-(4- | DMSO- |
| | chlorophenyl)acetamide | d₆): δ ppm |
| | | 1.48 (d, 6H), 3.75 (s, |
| | | 2H), 4.84 (m, |
| | | 1H), 6.57 (s, |
| | | 2H), 7.38 (m, |
| | | 3H), 7.84 (dd, |
| | | 1H), 8.18 (s, |
| | | 1H), 8.62 (d, |
| | | 1H), 8.69 (s, |
| | | 1H), 9.03 (s, |
| | | 1H), 10.80 (s, |
| | | 1H). |
| 88 | N-(2-{[2-amino-7-(propan-2-yl)-7H-pyrrolo[2,3- | MS m/z 493 |
| | d]pyrimidin-5-yl]carbonyl}pyridin-4-yl)-2-[3- | [M + H]⁺ |
| | (methylsulfon | |
| | yl)phenyl]acetamide | |
| 89 | N-(2-{[2-amino-7-(propan-2-yl)-7H-pyrrolo[2,3- | MS m/z 434 |
| | d]pyrimidin-5-yl]carbonyl}pyridin-4-yl)-2-(5- | [M + H]⁺ |
| | fluoropyridin-2-yl)acetamide | |
| 90 | N-(2-{[2-amino-7-(propan-2-yl)-7H-pyrrolo[2,3- | MS m/z |
| | d]pyrimidin-5-yl]carbonyl}pyridin-4-yl)-2-[4- | 474 [M + H]⁺ |
| | (trifluoromethyl)-1H-1,2,3-triazol-1-yl]acetamide | |
| 91 | N-(2-{[2-amino-7-(propan-2-yl)-7H-pyrrolo[2,3- | MS m/z |
| | d]pyrimidin-5-yl]carbonyl}pyridin-4-yl)-2-(4-cyclopropyl- | 446 [M + H]⁺ |
| | 1H-1,2,3-triazol-1-yl)acetamide | |

The following examples were prepared according to Method 1 (Example 1) using (4-aminopyridin-2-yl)[7-(2-{[tert-butyl(dimethyl)silyl]oxy}-1-{[(tert-butylsilyl)oxy]methyl}ethyl)-7H-pyrrolo[2,3-d]pyrimidin-5-yl]methanone (Preparation 32) and the appropriate acetic acids. The Examples were purified using preparative HPLC.

| Example | Name | Data |
|---|---|---|
| 92 | 2-(4-chlorophenyl)-N-(2-{[7-(1,3-dihydroxypropan-2-yl)-7H- | MS mz/ |
| | pyrrolo[2,3-d]pyrimidin-5-yl]carbonyl}pyridin-4- | 466 [M + H]⁺ |
| | yl)acetamide | |
| 93 | N-(2-{[7-(1,3-dihydroxypropan-2-yl)-7H-pyrrolo[2,3- | MS m/z |
| | d]pyrimidin-5-yl]carbonyl}pyridin-4-yl)-2-[4- | 500 [M + H]⁺ |
| | (trifluoromethyl)phenyl]acetamide | |
| 94 | N-(2-{[7-(1,3-dihydroxypropan-2-yl)-7H-pyrrolo[2,3- | MS m/z |
| | d]pyrimidin-5-yl]carbonyl}pyridin-4-yl)-2-[3-fluoro-4- | 518 [M + H]⁺ |
| | (trifluoromethyl)phenyl]acetamide | |
| 95 | 2-(2,4-dichlorophenyl)-N-(2-{[7-(1,3-dihydroxypropan-2-yl)- | MS m/z |
| | 7H-pyrrolo[2,3-d]pyrimidin-5-yl]carbonyl}pyridin-4- | 500 [M + H]⁺ |
| | yl)acetamide | |
| 96 | N-(2-{[7-(1,3-dihydroxypropan-2-yl)-7H-pyrrolo[2,3- | MS m/z |
| | d]pyrimidin-5-yl]carbonyl}pyridin-4-yl)-2-[3- | 500 [M + H]⁺ |
| | (trifluoromethyl)phenyl]acetamide | |
| 97 | N-(2-{[7-(1,3-dihydroxypropan-2-yl)-7H-pyrrolo[2,3- | MS m/z |
| | d]pyrimidin-5-yl]carbonyl}pyridin-4-yl)-2-[3- | 516 [M + H]⁺ |
| | (trifluoromethoxy)phenyl]acetamide | |
| 98 | N-(2-{[7-(1,3-dihydroxypropan-2-yl)-7H-pyrrolo[2,3- | MS m/z |
| | d]pyrimidin-5-yl]carbonyl}pyridin-4-yl)-2-[4- | 516 [M + H]⁺ |
| | (trifluoromethoxy)phenyl]acetamide | |
| 99 | 2-(3,4-dichlorophenyl)-N-(2-{[7-(1,3-dihydroxypropan-2-yl)- | MS m/z |
| | 7H-pyrrolo[2,3-d]pyrimidin-5-yl]carbonyl}pyridin-4- | 500 [M + H]⁺ |
| | yl)acetamide | |
| 100 | 2-(5-chloropyridin-2-yl)-N-(2-{[7-(1,3-dihydroxypropan-2- | MS m/z |
| | yl)-7H-pyrrolo[2,3-d]pyrimidin-5-yl]carbonyl}pyridin-4- | 467 [M + H]⁺ |
| | yl)acetamide | |
| 101 | N-(2-{[7-(1,3-dihydroxypropan-2-yl)-7H-pyrrolo[2,3- | MS m/z |
| | d]pyrimidin-5-yl]carbonyl}pyridin-4-yl)-2-[3- | 490 [M + H]⁺ |
| | (trifluoromethyl)-1H-pyrazol-1-yl]acetamide | |

The following examples were prepared according to Method 1 (Example 1) using (4-aminopyridin-2-yl)[7-(2-{[tert-butyl(dimethyl)silyl]oxy}-1-{[(tert-butylsilyl)oxy]methyl}-1-methylethyl)-7H-pyrrolo[2,3-d]pyrimidin-5-yl]methanone (Preparation 33) and the appropriate acetic acids. The Examples were purified using preparative HPLC.

| Example | Name | Data |
|---|---|---|
| 102 | 2-(4-chlorophenyl)-N-(2-{[7-(1,3-dihydroxy-2- | MS m/z |
| | methylpropan-2-yl)-7H-pyrrolo[2,3-d]pyrimidin-5- | 480 [M + H]⁺ |
| | yl]carbonyl}pyridin-4-yl)acetamide | |
| 103 | N-(2-{[7-(1,3-dihydroxy-2-methylpropan-2-yl)-7H- | MS m/z |
| | pyrrolo[2,3-d]pyrimidin-5-yl]carbonyl}pyridin-4-yl)-2-[4- | 514 [M + H]⁺ |
| | (trifluoromethyl)phenyl]acetamide | |
| 104 | N-(2-{[7-(1,3-dihydroxy-2-methylpropan-2-yl)-7H- | MS m/z |
| | pyrrolo[2,3-d]pyrimidin-5-yl]carbonyl}pyridin-4-yl)-2-[3- | 514 [M + H]⁺ |
| | (trifluoromethyl)phenyl]acetamide | |
| 105 | N-(2-{[7-(1,3-dihydroxy-2-methylpropan-2-yl)-7H- | MS m/z |
| | pyrrolo[2,3-d]pyrimidin-5-yl]carbonyl}pyridin-4-yl)-2-[3- | 504 [M + H]⁺ |
| | (trifluoromethyl)-1H-pyrazol-1-yl]acetamide | |
| 106 | 2-(5-chloropyridin-2-yl)-N-(2-{[7-(1,3-dihydroxy-2- | MS m/z |
| | methylpropan-2-yl)-7H-pyrrolo[2,3-d]pyrimidin-5- | 481 [M + H]⁺ |
| | yl]carbonyl}pyridin-4-yl)acetamide | |
| 107 | 2-(4-cyanophenyl)-N-(2-{[7-(1,3-dihydroxy-2- | MS m/z |
| | methylpropan-2-yl)-7H-pyrrolo[2,3-d]pyrimidin-5- | 471 [M + H]⁺ |
| | yl]carbonyl}pyridin-4-yl)acetamide | |
| 108 | N-(2-{[7-(1,3-dihydroxy-2-methylpropan-2-yl)-7H- | MS m/z |
| | pyrrolo[2,3-d]pyrimidin-5-yl]carbonyl}pyridin-4-yl)-2-[5- | 518 [M + H]⁺ |
| | methyl-3-(trifluoromethyl)-1H-pyrazol-1-yl]acetamide | |

### Example 109

### 2-(5-Cyanopyridin-2-yl)-N-{2-[(7-isopropyl-7H-pyrrolo[2,3-d]pyrimidin-5-yl)carbonyl]pyridin-4-yl}acetamide

The title compound was prepared according to the method described for Example 62 using 2-(5-cyanopyridin-2-yl)-N-{2-[(7-isopropyl-7H-pyrrolo[2,3-d]pyrimidin-5-yl)carbonyl]pyridin-4-yl}acetamide (Example 110) to afford the title compound as a white solid in 38% yield, 37 mg.

LCMS (System 9): Rt=2.82 minutes MS m/z 426 [M+H]⁺

### Example 110

### 2-(5-Cyanopyridin-2-yl)-N-{2-[(7-isopropyl-7H-pyrrolo[2,3-d]pyrimidin-5-yl)carbonyl]pyridin-4-yl}acetamide

The title compound was prepared according to the method described for Method 3 (Example 66) using (4-aminopyridin-2-yl)(7-isopropyl-7H-pyrrolo[2,3-d]pyrimidin-5-yl)methanone (Preparation 17) and (5-bromopyridin-2-yl)acetic acid to afford the title compound as a yellow solid in 23% yield, 120 mg.

LCMS (System 9): Rt=3.17 minutes MS m/z 479 [M+H]⁺

### Example 111

### 2-(4-cyanophenyl)-N-(4-{[7-(1-hydroxy-2-methylpropan-2-yl)-7H-pyrrolo[2,3-d]pyrimidin-5-yl]carbonyl}pyridin-2-yl)acetamide

Aqueous HCl (2.0 M, 0.5 mL) was added to 2-(4-cyanophenyl)-N-(4-{[7-(2-{[tert-butyl(dimethyl)silyl]oxy}-1,1-dimethylethyl)-7H-pyrrolo[2,3-d]pyrimidin-5-yl]carbonyl}pyridin-2-yl)acetamide (Preparation 4, 26 mg, 0.08 mmol) in THF (0.5 mL) and the mixture was stirred at room temperature for 16 hours. Aqueous sodium hydroxide (2.0 M) was added to neutralize the reaction mixture, then DCM (3 mL) was added. The organic phase was dried over magnesium sulphate and evaporated in vacuo. The crude solid was purified by silica gel column chromatography eluting with a gradient of DCM:MeOH:cNH₃ 95:5:0.5 to 90:10:1 to afford the title compound as a yellow solid in 18% yield, 4 mg.

LCMS (system 1): Rt=2.56 minutes MS m/z 455 [M+H]⁺

### Example 112

### 2-(3,5-Dimethyl-1H-pyrazol-1-yl)-N-[4-({7-[2-hydroxy-1-(hydroxymethyl)ethyl]-7H-pyrrolo[2,3-d]pyrimidin-5-yl}carbonyl)pyridin-2-yl]acetamide

The title compound was prepared according to the method described for Example 111 using 2-(3,5-dimethyl-1H-pyrazol-1-yl)-N-[4-({7-[2-tert-butyl(dimethyl)silyloxy-1-(tert-butyl(dimethyl)silyloxymethyl)ethyl]-7H-pyrrolo[2,3-d]pyrimidin-5-yl}carbonyl)pyridin-2-yl]acetamide (Preparation 3) to afford the title compound as a white solid in 43% yield, 70 mg.

LCMS (system 1): Rt=1.62 minutes MS m/z 450 [M+H]⁺

### Example 113

### 2-(4-Chlorophenyl)-N-[4-({7-[2-hydroxy-1-(hydroxymethyl)ethyl]-7H-pyrrolo[2,3-d]pyrimidin-5-yl}carbonyl)pyridin-2-yl]acetamide

4-Chlorophenylacetic acid (13 mg, 0.07 mmol) was added to (2-aminopyridin-4-yl)[7-(2,2-dimethyl-1,3-dioxan-5-yl)-7H-pyrrolo[2,3-d]pyrimidin-5-yl]methanone (Preparation 27, 20 mg, 0.06 mmol) and HATU (33 mg, 0.09 mmol) in pyridine (1 mL). The mixture was stirred at 50° C. for 16 hours. Saturated aqueous sodium bicarbonate (1 mL) was added, then extracted with ethyl acetate (5 mL). The organic phase was dried over magnesium sulphate and evaporated in vacuo. The crude solid was dissolved in DCM (1 mL) and trifluoroacetic acid (1 mL) was added. The mixture was stirred at room temperature for 30 minutes then evaporated in vacuo and purified by preparative HPLC to afford the title compound in 43% yield, 12 mg.

LCMS (system 4): Rt=2.66 minutes MS m/z 466, 468 [M+H]⁺

### Example 114

### 2-(4-cyanophenyl)-N-(4-{[7-(1,3-dihydroxypropan-2-yl)-7H-pyrrolo[2,3-d]pyrimidin-5-yl]carbonyl}pyridin-2-yl)acetamide

Prepared according to Example 113 using 4-cyanophenylacetic acid.

LCMS Rt=2.55 minutes, MS m/z 457 [M+H]⁺

### Example 115

### 2-(4-Chlorophenyl)-N-{4-[(7-{[3-(hydroxymethyl)oxetan-3-yl]methyl}-7H-pyrrolo[2,3-d]pyrimidin-5-yl)carbonyl}pyridin-2-yl]acetamide

Tetrabutylammonium fluoride (0.07 mL, 0.07 mmol, 1M in THF) was added to N-{4-[(7-{[3-({[tert-butyl(dimethyl)silyl]oxy}methyl)oxetan-3-yl]methyl}-7H-pyrrolo[2,3-d]pyrimidin-5-yl)carbonyl]pyridin-2-yl}-2-(4-chlorophenyl)acetamide (Preparation 2, 36 mg, 0.06 mmol) in THF (2 mL). The reaction mixture was stirred at room temperature for 30 minutes then water (2 mL) was added. The mixture was extracted with EtOAc (3×5 mL) and the combined organic phases were washed with saturated aqueous sodium chloride (5 mL). The organic phase was separated though a phase separation cartridge, evaporated in vacuo and the crude residue was purified by preparative HPLC to afford the title compound as a white solid in 30% yield, 9 mg.

LCMS (system 1): Rt=2.26 minutes MS m/z 490, 492 [M+H]⁺

### Library Protocol 1

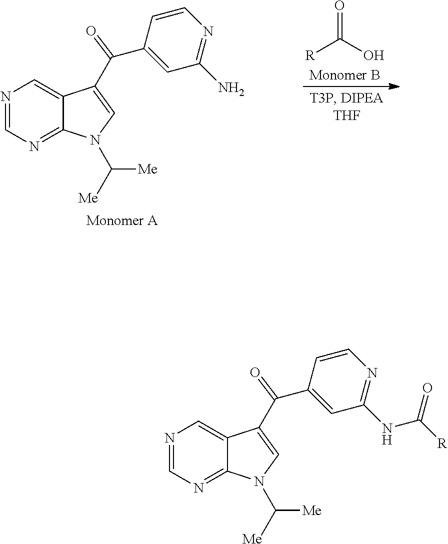

To a 0.2 M solution of Monomer A ((2-aminopyridin-4-yl)[7-(propan-2-yl)-7H-pyrrolo[2,3-d]pyrimidin-5-yl]methanone (Preparation 24, 500 uL, 100 umol) in anhydrous THF was added Monomer B (130 umol) followed by T3P (300 umol). DIPEA (350 umol) was added and the reaction stirred at 25° C. for 16 hours. The reaction was concentrated in vacuo and the residue dissolved in DMSO (1 mL) and purified using preparative HPLC.

### Purification Method:

Column: Waters Purification System 50×19 mm Xbridge C18 column; 5 um, mobile phase A: acetonitrile; Mobile phase B: 0.05% ammonia in water. Initial gradient 10% A, End gradient either 50%, 70%, 80% or 90% A. Gradient time: 7 minutes. Flow rate: 20 mL/min.

| Example | Name/Structure | Data |
|---|---|---|
| 116 | N-(4-{[7-(propan-2-yl)-7H-pyrrolo[2,3-d]pyrimidin-5- | MS m/z |
| | yl]carbonyl}pyridin-2-yl)-2-[4-(trifluoromethyl)-1H-1,2,3- | 459 [M + H]⁺ |
| | triazol-1-yl]acetamide | |
| 117 | 2-[4-(methoxymethyl)-1H-1,2,3-triazol-1-yl]-N-(4-{[7- | MS m/z |
| | (propan-2-yl)-7H-pyrrolo[2,3-d]pyrimidin-5- | 435 [M + H]⁺ |
| | yl]carbonyl}pyridin-2-yl)acetamide | |
| 118 | 2-(2,4-difluorophenyl)-N-(4-{[7-(propan-2-yl)-7H- | MS m/z |
| | pyrrolo[2,3-d]pyrimidin-5-yl]carbonyl}pyridin-2-yl)acetamide | 436 [M + H]⁺ |
| 119 | N-(4-{[7-(propan-2-yl)-7H-pyrrolo[2,3-d]pyrimidin-5- | MS m/z |
| | yl]carbonyl}pyridin-2-yl)-2-[2- | 484 [M + H]⁺ |
| | (trifluoromethoxy)phenyl]acetamide | |
| 120 | N-(4-{[7-(propan-2-yl)-7H-pyrrolo[2,3-d]pyrimidin-5- | MS m/z |
| | yl]carbonyl}pyridin-2-yl)-2-[4- | 484 [M + H]⁺ |
| | (trifluoromethoxy)phenyl]acetamide | |
| 121 | 2-(4-methyl-1H-1,2,3-triazol-1-yl)-N-(4-{[7-(propan-2-yl)-7H- | MS m/z |
| | pyrrolo[2,3-d]pyrimidin-5-yl]carbonyl}pyridin-2-yl)acetamide | 405 [M + H]⁺ |
| 122 | N-(4-{[7-(propan-2-yl)-7H-pyrrolo[2,3-d]pyrimidin-5- | MS m/z |
| | yl]carbonyl}pyridin-2-yl)-2-[4- | 468 [M + H]⁺ |
| | (trifluoromethyl)phenyl]acetamide | |
| 123 | 2-[3-(methylsulfonyl)phenyl]-N-(4-{[7-(propan-2-yl)-7H- | MS m/z |
| | pyrrolo[2,3-d]pyrimidin-5-yl]carbonyl}pyridin-2-yl)acetamide | 478 [M + H]⁺ |
| 124 | 2-[3-fluoro-4-(trifluoromethyl)phenyl]-N-(4-{[7-(propan-2-yl)- | MS m/z |
| | 7H-pyrrolo[2,3-d]pyrimidin-5-yl]carbonyl}pyridin-2- | 486 [M + H]⁺ |
| | yl)acetamide | |

### Example 125

### N-(4-{[2-hydroxy-7-(propan-2-yl)-7H-pyrrolo[2,3-d]pyrimidin-5-yl]carbonyl}pyridin-2-yl)-2-[5-methyl-3-(trifluoromethyl)-1H-pyrazol-1-yl]acetamide

To a solution of (2-aminopyridin-4-yl)(2-hydroxy-7-isopropyl-7H-pyrrolo[2,3-d]pyrimidin-5-yl)methanone (Preparation 26, 20 mg, 0.0667 mmol) in DMF (1 mL) was added 5-methyl-3-(trifluoromethyl)-1H-pyrazol-1-yl]acetic acid (14 mg, 0.070 mmol), HATU (50 mg, 0.134 mmol) and DMAP (49 mg, 0.402 mmol) and the reaction was stirred at room temperature for 16 hours. The reaction was diluted with EtOAc, washed with saturated aqueous NaHCO₃ solution, water, brine, dried over MgSO₄ and concentrated in vacuo. The residue was purified using preparative TLC eluting with 70% MeOH in DCM to afford the title compound.

LCMS Rt=2.92 minutes MS m/z 488 [M+H]⁺

### Example 126

### 2-(5-Bromopyridin-2-yl)-N-{4-[(7-isopropyl-7H-pyrrolo[2,3-d]pyrimidin-5-yl)carbonyl}pyridin-2-yl]acetamide

The title compound was prepared according to the method described for Method 1 (Example 1) using (2-aminopyridin-4-yl)(7-isopropyl-7H-pyrrolo[2,3-d]pyrimidin-5-yl)methanone (Preparation 24) and (5-bromopyridin-2-yl)acetic acid (Preparation 133) to afford the title compound as a white solid in 70% yield, 350 mg.

LCMS (System 9): Rt=4.78 minutes MS m/z 480 [M+H]⁺

### Example 127

### 2-(3,5-dimethyl-1H-pyrazol-1-yl)-N-(4-{[7-(2-hydroxy-2-methylpropyl)-7H-pyrrolo[2,3-d]pyrimidin-5-yl]carbonyl}pyridin-2-yl)acetamide

The title compound was prepared according to the method described for Example 178 using 2-(3,5-dimethyl-1H-pyrazol-1-yl)-N-[4-(7H-pyrrolo[2,3-d]pyrimidin-5-ylcarbonyl)pyridin-2-yl]acetamide (Preparation 108) and 2,2-dimethyloxirane. Purified using preparative HPLC.

LCMS Rt=2.60 minutes MS m/z 448 [M+H]⁺

### Library Protocol 2

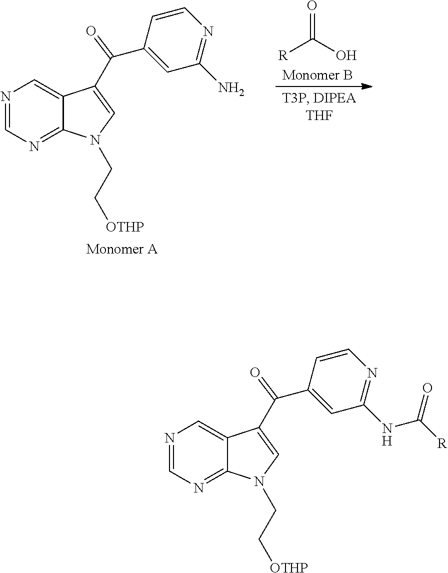

To a 0.2 M solution of Monomer A ((2-aminopyridin-4-yl){7-[2-(tetrahydro-2H-pyran-2-yloxy)ethyl]-7H-pyrrolo[2,3-d]pyrimidin-5-yl}methanone (Preparation 34, 500 uL, 100 umol) in anhydrous THF was added Monomer B (130 umol) followed by T3P (300 umol). DIPEA (350 umol) was added and the reaction stirred at 25° C. for 16 hours. The reaction was concentrated in vacuo and the residue dissolved in DMSO (1 mL) and purified using preparative HPLC.

### Purification Method:

Column: Waters Purification System 50×19 mm Xbridge C18 column; 5 um, mobile phase A: acetonitrile; Mobile phase B: 0.05% ammonia in water. Initial gradient 10% A, End gradient either 40%, 50%, 60% or 80% A. Gradient time: 7 minutes. Flow rate: 20 mL/min.

| Example | Name/Structure | Data |
|---|---|---|
| 128 | N-(4-{[7-(2-hydroxyethyl)-7H-pyrrolo[2,3-d]pyrimidin-5- | MS m/z |
| | yl]carbonyl}pyridin-2-yl)-2-(3-methyl-1H-pyrazol-1- | 406 [M + H]⁺ |
| | yl)acetamide | |
| 129 | N-(4-{[7-(2-hydroxyethyl)-7H-pyrrolo[2,3-d]pyrimidin-5- | MS m/z |
| | yl]carbonyl}pyridin-2-yl)-2-[4-(propan-2-yl)-1H-1,2,3- | 435 [M + H]⁺ |
| | triazol-1-yl]acetamide | |
| 130 | N-(4-{[7-(2-hydroxyethyl)-7H-pyrrolo[2,3-d]pyrimidin-5- | MS m/z |
| | yl]carbonyl}pyridin-2-yl)-2-(5-methyl-1H-pyrazol-1- | 406 [M + H]⁺ |
| | yl)acetamide | |
| 131 | N-(4-{[7-(2-hydroxyethyl)-7H-pyrrolo[2,3-d]pyrimidin-5- | MS m/z |
| | yl]carbonyl}pyridin-2-yl)-2-[3- | 480 [M + H]⁺ |
| | (methylsulfonyl)phenyl]acetamide | |
| 132 | N-(4-{[7-(2-hydroxyethyl)-7H-pyrrolo[2,3-d]pyrimidin-5- | MS m/z |
| | yl]carbonyl}pyridin-2-yl)-2-[5-methyl-3-(trifluoromethyl)- | 474 [M + H]⁺ |
| | 1H-pyrazol-1-yl]acetamide | |
| 133 | N-(4-{[7-(2-hydroxyethyl)-7H-pyrrolo[2,3-d]pyrimidin-5- | MS m/z |
| | yl]carbonyl}pyridin-2-yl)-2-(4-methyl-1H-1,2,3-triazol-1- | 407 [M + H]⁺ |
| | yl)acetamide | |
| 134 | N-(4-{[7-(2-hydroxyethyl)-7H-pyrrolo[2,3-d]pyrimidin-5- | MS m/z |
| | yl]carbonyl}pyridin-2-yl)-2-[2- | 486 [M + H]⁺ |
| | (trifluoromethoxy)phenyl]acetamide | |
| 135 | N-(4-{[7-(2-hydroxyethyl)-7H-pyrrolo[2,3-d]pyrimidin-5- | MS m/z |
| | yl]carbonyl}pyridin-2-yl)-2-[3- | 486 [M + H]⁺ |
| | (trifluoromethoxy)phenyl]acetamide | |
| 136 | 2-(4-chlorophenyl)-N-(4-{[7-(2-hydroxyethyl)-7H- | MS m/z |
| | pyrrolo[2,3-d]pyrimidin-5-yl]carbonyl}pyridin-2- | 436 [M + H]⁺ |
| | yl)acetamide | |
| 137 | N-(4-{[7-(2-hydroxyethyl)-7H-pyrrolo[2,3-d]pyrimidin-5- | MS m/z |
| | yl]carbonyl}pyridin-2-yl)-2-[4-(trifluoromethyl)-1H-1,2,3- | 461 [M + H]⁺ |
| | triazol-1-yl]acetamide | |
| 138 | 2-(3,4-dichlorophenyl)-N-(4-{[7-(2-hydroxyethyl)-7H- | MS m/z |
| | pyrrolo[2,3-d]pyrimidin-5-yl]carbonyl}pyridin-2- | 470 [M + H]⁺ |
| | yl)acetamide | |
| 139 | 2-(3,5-dimethyl-1H-pyrazol-1-yl)-N-(4-{[7-(2- | MS m/z |
| | hydroxyethyl)-7H-pyrrolo[2,3-d]pyrimidin-5- | 420 [M + H]⁺ |
| | yl]carbonyl}pyridin-2-yl)acetamide | |
| 140 | 2-(2,4-difluorophenyl)-N-(4-{[7-(2-hydroxyethyl)-7H- | MS m/z |
| | pyrrolo[2,3-d]pyrimidin-5-yl]carbonyl}pyridin-2- | 438 [M + H]⁺ |
| | yl)acetamide | |
| 141 | N-(4-{[7-(2-hydroxyethyl)-7H-pyrrolo[2,3-d]pyrimidin-5- | MS m/z |
| | yl]carbonyl}pyridin-2-yl)-2-[4- | 470 [M + H]⁺ |
| | (trifluoromethyl)phenyl]acetamide | |
| 142 | 2-[3-fluoro-4-(trifluoromethyl)phenyl]-N-(4-{[7-(2- | MS m/z |
| | hydroxyethyl)-7H-pyrrolo[2,3-d]pyrimidin-5- | 488 [M + H]⁺ |
| | yl]carbonyl}pyridin-2-yl)acetamide | |
| 143 | 2-(4-cyanophenyl)-N-(4-{[7-(2-hydroxyethyl)-7H- | MS m/z |
| | pyrrolo[2,3-d]pyrimidin-5-yl]carbonyl}pyridin-2- | 427 [M + H]⁺ |
| | yl)acetamide | |
| 144 | N-(4-{[7-(2-hydroxyethyl)-7H-pyrrolo[2,3-d]pyrimidin-5- | MS m/z |
| | yl]carbonyl}pyridin-2-yl)-2-[4- | 486 [M + H]⁺ |
| | (trifluoromethoxy)phenyl]acetamide | |
| 145 | 2-(4-fluorophenyl)-N-(4-{[7-(2-hydroxyethyl)-7H- | MS m/z |
| | pyrrolo[2,3-d]pyrimidin-5-yl]carbonyl}pyridin-2- | 420 [M + H]⁺ |
| | yl)acetamide | |
| 146 | 2-(3-chlorophenyl)-N-(4-{[7-(2-hydroxyethyl)-7H- | MS m/z 436 |
| | pyrrolo[2,3-d]pyrimidin-5-yl]carbonyl}pyridin-2- | |
| | yl)acetamide | |

### Library Protocol 3

### Example 147

### 2-[5-methyl-3-(trifluoromethyl)-1H-pyrazol-1-yl]-N-(4-{[7-(propan-2-yl)-7H-pyrrolo[2,3-d]pyrimidin-5-yl]carbonyl}pyridin-2-yl)acetamide

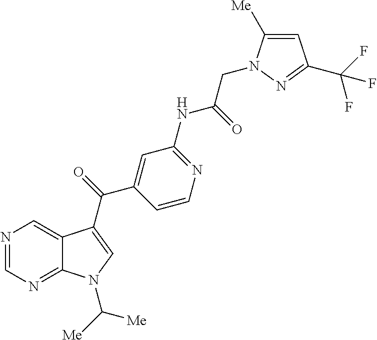 [SMILES: Cc1cc(C(F)(F)F)nn1CC(=O)Nc1cc(C(=O)c2cn(C(C)C)c3ncncc23)ccn1]

A 0.15M solution of (2-Aminopyridin-4-yl)(7-isopropyl-7H-pyrrolo[2,3-d]pyrimidin-5-yl)methanone (Preparation 24, 500 uL, 75 umol) in DMA was added to [5-methyl-3-(trifluoromethyl)-1H-pyrazol-1-yl]acetic acid (75 uM) followed by NMM (25 uL, 150 umol), DMAP (3.5 umol) and a 0.15M solution of HATU (500 uL, 75 umol) in DMA. The reaction was shaken at 50° C. for 16 hours. The reaction was concentrated in vacuo and purified using preparative HPLC (Purification method 2 below, initial gradient 25% A, final gradient 55% A) to obtain the title compound as the TFA salt.

LCMS Rt=2.85 minutes MS m/z 472 [M+H]⁺

The following compounds were prepared as described above for Example 147 using (2-aminopyridin-4-yl)(7-isopropyl-7H-pyrrolo[2,3-d]pyrimidin-5-yl)methanone (Preparation 24) and the appropriate acid with purification using preparative HPLC using one of the two methods below (initial and final gradients for each compound are reported in the table).

### Purification Method 1:

Column: YMC-pack ODS-AQ 150×30 mm, 5 um, mobile phase A: acetonitrile; Mobile phase B: 0.1% TFA in water. Gradient time: 8 minutes. Flow rate: 35 mL/min. Initial/end gradients are reported for each compound below.

### Purification Method 2:

Column: Sepax BR-C18 100×21.2 mm, 5 um, mobile phase A: acetonitrile; Mobile phase B: 0.1% TFA in water. Gradient time: 8 minutes. Flow rate 30 mL/min. Initial/end gradients are reported for each compound below.

| Example | Name | Data |
|---|---|---|
| 148 | 2-[5-(propan-2-yl)-1H-pyrazol-1-yl]-N-(4-{[7-(propan-2-yl)- | MS m/z |
| | 7H-pyrrolo[2,3-d]pyrimidin-5-yl]carbonyl}pyridin-2- | 432 [M + H]⁺ |
| | yl)acetamide | |
| 149 | 2-phenyl-N-(4-{[7-(propan-2-yl)-7H-pyrrolo[2,3- | MS m/z |
| | d]pyrimidin-5-yl]carbonyl}pyridin-2-yl)acetamide | 400 [M + H]⁺ |
| 150 | 2-(4-chlorophenyl)-N-(4-{[7-(propan-2-yl)-7H-pyrrolo[2,3- | MS m/z |
| | d]pyrimidin-5-yl]carbonyl}pyridin-2-yl)acetamide | 430 [M + H]⁺ |
| 151 | 2-[5-methyl-3-(propan-2-yl)-1H-pyrazol-1-yl]-N-(4-{[7- | MS m/z |
| | (propan-2-yl)-7H-pyrrolo[2,3-d]pyrimidin-5- | 446 [M + H]⁺ |
| | yl]carbonyl}pyridin-2-yl)acetamide | |
| 152 | 2-(3,5-dimethyl-1H-pyrazol-1-yl)-N-(4-{[7-(propan-2-yl)- | MS m/z |
| | 7H-pyrrolo[2,3-d]pyrimidin-5-yl]carbonyl}pyridin-2- | 418 [M + H]⁺ |
| | yl)acetamide | |
| 153 | 2-(4-fluorophenyl)-N-(4-{[7-(propan-2-yl)-7H-pyrrolo[2,3- | MS m/z |
| | d]pyrimidin-5-yl]carbonyl}pyridin-2-yl)acetamide | 418 [M + H]⁺ |
| 154 | N-(4-{[7-(propan-2-yl)-7H-pyrrolo[2,3-d]pyrimidin-5- | MS m/z |
| | yl]carbonyl}pyridin-2-yl)-2-[3- | 484 [M + H]⁺ |
| | (trifluoromethoxy)phenyl]acetamide | |
| 155 | 2-(3-chlorophenyl)-N-(4-{[7-(propan-2-yl)-7H-pyrrolo[2,3- | MS m/z |
| | d]pyrimidin-5-yl]carbonyl}pyridin-2-yl)acetamide | 434 [M + H]⁺ |
| 156 | 2-(1H-imidazol-1-yl)-N-(4-{[7-(propan-2-yl)-7H- | MS m/z |
| | pyrrolo[2,3-d]pyrimidin-5-yl]carbonyl}pyridin-2- | 390 [M + H]⁺ |
| | yl)acetamide | |
| 157 | 2-(2,4-dichlorophenyl)-N-(4-{[7-(propan-2-yl)-7H- | MS m/z |
| | pyrrolo[2,3-d]pyrimidin-5-yl]carbonyl}pyridin-2- | 468 [M + H]⁺ |
| | yl)acetamide | |
| 158 | N-(4-{[7-(propan-2-yl)-7H-pyrrolo[2,3-d]pyrimidin-5- | MS m/z |
| | yl]carbonyl}pyridin-2-yl)-2-(1H-pyrazol-1-yl)acetamide | 390 [M + H]⁺ |
| 159 | 2-(2-chlorophenyl)-N-(4-{[7-(propan-2-yl)-7H-pyrrolo[2,3- | MS m/z |
| | d]pyrimidin-5-yl]carbonyl}pyridin-2-yl)acetamide | 434 [M + H]⁺ |
| 160 | 2-(5-methyl-1H-pyrazol-1-yl)-N-(4-{[7-(propan-2-yl)-7H- | MS m/z |
| | pyrrolo[2,3-d]pyrimidin-5-yl]carbonyl}pyridin-2- | 404 [M + H]⁺ |
| | yl)acetamide | |
| 161 | 2-(4-methoxyphenyl)-N-(4-{[7-(propan-2-yl)-7H- | MS m/z |
| | pyrrolo[2,3-d]pyrimidin-5-yl]carbonyl}pyridin-2- | 430 [M + H]⁺ |
| | yl)acetamide | |
| 162 | 2-(2-methyl-1H-imidazol-1-yl)-N-(4-{[7-(propan-2-yl)-7H- | MS m/z |
| | pyrrolo[2,3-d]pyrimidin-5-yl]carbonyl}pyridin-2- | 404 [M + H]⁺ |
| | yl)acetamide | |
| 163 | N-(4-{[7-(propan-2-yl)-7H-pyrrolo[2,3-d]pyrimidin-5- | MS m/z |
| | yl]carbonyl}pyridin-2-yl)-2-(pyridin-2-yl)acetamide | 401 [M + H]⁺ |
| 164 | N-(4-{[7-(propan-2-yl)-7H-pyrrolo[2,3-d]pyrimidin-5- | MS m/z |
| | yl]carbonyl}pyridin-2-yl)-2-[4-(propan-2-yl)-1H-1,2,3- | 433 [M + H]⁺ |
| | triazol-1-yl]acetamide | |

### Example 165

### 2-(4-cyanophenyl)-N-(6-methoxy-4-{[7-(propan-2-yl)-7H-pyrrolo[2,3-d]pyrimidin-5-yl]carbonyl}pyridin-2-yl)acetamide

A mixture of (2-chloro-6-methoxypyridin-4-yl)(7-isopropyl-7H-pyrrolo[2,3-d]pyrimidin-5-yl)methanone (Preparation 45, 50 mg, 0.15 mmol), (4-cyanophenyl)acetamide (J. Med. Chem. 52(10), 3366, (2009), 24 mg, 0.15 mmol), Pd(dba)₂ (17 mg, 0.03 mmol), xantphos (28 mg, 0.048 mmol) and cesium carbonate (73 mg, 0.226 mmol) in DMF (5 mL) was heated under nitrogen to 120° C. for 18 hours. The reaction was partitioned between EtOAc (10 mL) and water (10 mL), dried over MgSO₄ and concentrated in vacuo. The residue was purified using preparative HPLC to afford the title compound.

LCMS Rt=3.28 minutes MS m/z 455 [M+H]⁺

### Example 166

### N-(2-{[7-(3-methyloxetan-3-yl)-7H-pyrrolo[2,3-d]pyrimidin-5-yl]carbonyl}pyridin-4-yl)-2-[3-(trifluoromethyl)phenyl]acetamide

### Method 4

To a solution of (4-aminopyridin-2-yl)[7-(3-methyloxetan-3-yl)-7H-pyrrolo[2,3-d]pyrimidin-5-yl]methanone (Preparation 19, 31 mg, 0.10 mmol) in pyridine (0.5 mL) was added HATU (57 mg, 0.15 mmol) and 3-trifluoromethylphenylacetic acid (26.5 mg, 0.13 mmol) and the reaction stirred at 50° C. for 18 hours. The reaction was cooled to room temperature and diluted with saturated aqueous NaHCO₃ solution, extracted with EtOAc (3×5 mL), the organic layers combined, washed with brine, dried over MgSO₄ and concentrated in vacuo. The residue was dissolved in DMSO (1 mL) and purified using preparative HPLC to afford the title compound as a white solid.

LCMS Rt=3.36 minutes MS m/z 496 [M+H]⁺

The following Examples were prepared according to Method 4 using (4-aminopyridin-2-yl)[7-(3-methyloxetan-3-yl)-7H-pyrrolo[2,3-d]pyrimidin-5-yl]methanone (Preparation 19) and the appropriate acetic acids.

| Example | Name | Data |
|---|---|---|
| 167 | N-(2-{[7-(3-methyloxetan-3-yl)-7H-pyrrolo[2,3-d]pyrimidin- | MS m/z |
| | 5-yl]carbonyl}pyridin-4-yl)-2-[4- | 496 [M + H]⁺ |
| | (trifluoromethyl)phenyl]acetamide | |
| 168 | 2-(5-chloropyridin-2-yl)-N-(2-{[7-(3-methyloxetan-3-yl)- | MS m/z |
| | 7H-pyrrolo[2,3-d]pyrimidin-5-yl]carbonyl}pyridin-4- | 463 [M + H]⁺ |
| | yl)acetamide | |
| 169 | 2-(4-cyanophenyl)-N-(2-{[7-(3-methyloxetan-3-yl)-7H- | MS m/z |
| | pyrrolo[2,3-d]pyrimidin-5-yl]carbonyl}pyridin-4- | 453 [M + H]⁺ |
| | yl)acetamide | |
| 170 | 2-[3-fluoro-4-(trifluoromethyl)phenyl]-N-(2-{[7-(3- | MS m/z |
| | methyloxetan-3-yl)-7H-pyrrolo[2,3-d]pyrimidin-5- | 514 [M + H]⁺ |
| | yl]carbonyl}pyridin-4-yl)acetamide | |
| 171 | N-(2-{[7-(3-methyloxetan-3-yl)-7H-pyrrolo[2,3-d]pyrimidin- | MS m/z |
| | 5-yl]carbonyl}pyridin-4-yl)-2-[4-(trifluoromethyl)-1H-1,2,3- | 487 [M + H]+ |
| | triazol-1-yl]acetamide | |
| 172 | 2-(5-fluoropyridin-2-yl)-N-(2-{[7-(3-methyloxetan-3-yl)-7H- | MS m/z |
| | pyrrolo[2,3-d]pyrimidin-5-yl]carbonyl}pyridin-4- | 450 [M + H]+ |
| | yl)acetamide | |

### Example 173

### N-{4-[(7-tert-butyl-7H-pyrrolo[2,3-d]pyrimidin-5-yl)carbonyl}pyridin-2-yl]-2-(4-cyanophenyl)acetamide

Prepared according to the method described for Method 4 (Example 166) using (2-aminopyridin-4-yl)(7-tert-butyl-7H-pyrrolo[2,3-d]pyrimidin-5-yl)methanone (Preparation 28) and 4-cyanophenylacetic acid.

LCMS (system 3): Rt=3.20 minutes MS m/z 439 [M+H]⁺

### Example 174

### N-{4-[(7-tert-butyl-7H-pyrrolo[2,3-d]pyrimidin-5-yl)carbonyl}pyridin-2-yl]-2-(3,5-dimethyl-1H-pyrazol-1-yl)acetamide

Prepared according to the method described for Method 4 (Example 166) using (2-aminopyridin-4-yl)(7-tert-butyl-7H-pyrrolo[2,3-d]pyrimidin-5-yl)methanone (Preparation 28) and (3,5-dimethyl-1H-pyrazol-1-yl)acetic acid.

LCMS (system 1): Rt=2.81 minutes MS m/z 432 [M+H]⁺

### Example 175

### 2-(4-cyanophenyl)-N-{4-[(7-cyclopropyl-7H-pyrrolo[2,3-d]pyrimidin-5-yl)carbonyl]pyridin-2-yl}acetamide

Prepared according to the method described for Method 4 (Example 166) using (2-aminopyridin-4-yl)(7-cyclopropyl-7H-pyrrolo[2,3-d]pyrimidin-5-yl)methanone (Preparation 18) and 4-cyanophenylacetic acid to afford the title compound after preparative HPLC.

LCMS (System 3): Rt=2.83 minutes MS m/z 423 [M+H]⁺

### Example 176

### N-(4-{[7-(Cyclopropylmethyl)-7H-pyrrolo[2,3-d]pyrimidin-5-yl]carbonyl}pyridin-2-yl)-2-(3,5-dimethyl-1H-pyrazol-1-yl)acetamide

Cyclopropylmethyl bromide (30 mg, 0.22 mmol) was added to a mixture of 2-(3,5-dimethyl-1H-pyrazol-1-yl)-N-[4-(7H-pyrrolo[2,3-d]pyrimidin-5-ylcarbonyl)pyridin-2-yl]acetamide (Preparation 108, 75 mg, 0.20 mmol) and potassium carbonate (30 mg, 0.22 mmol) in DMF (1 mL). The mixture was stirred at room temperature for 16 hours. Saturated aqueous ammonium chloride (10 mL) was added to the reaction mixture. The mixture was extracted with DCM (3×10 mL) and the combined organic phases were dried over magnesium sulphate and evaporated in vacuo. The crude solid was purified by silica gel column chromatography eluting with a gradient of EtOAc:MeOH 100:0 to 90:10 to afford the title compound as a white solid in 71% yield, 61 mg.

LCMS (system 1): Rt=2.27 minutes MS m/z 430 [M+H]⁺

### Example 177

### 2-(3,5-Dimethyl-1H-pyrazol-1-yl)-N-[4-({7-[(1-hydroxycyclopropyl)methyl]-7H-pyrrolo[2,3-d]pyrimidin-5-yl}carbonyl)pyridin-2-yl]acetamide

The title compound was prepared according to the method described for Example 176 using 2-(3,5-dimethyl-1H-pyrazol-1-yl)-N-[4-(7H-pyrrolo[2,3-d]pyrimidin-5-ylcarbonyl)pyridin-2-yl]acetamide (Preparation 108) and 1-oxa-spiro[2.2]pentane (Preparation 122). Purified using preparative HPLC.

LCMS (System 3): Rt=1.34 minutes MS m/z 446 [M+H]⁺

### Preparation 1

### N-(5-{2-Amino-7-[2-(tert-butyl-dimethyl-silanyloxy)-1,1-dimethyl-ethyl]-7H-pyrrolo[2,3-d]pyrimidine-5-carbonyl}-2-cyano-phenyl)-2-(4-trifluoromethyl-phenyl)-acetamide

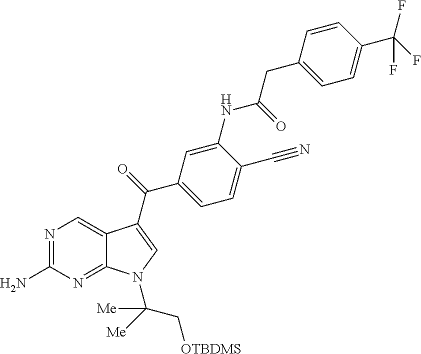 [SMILES: CC(C)(CO[Si](C)(C)C(C)(C)C)n1cc(C(=O)c2ccc(C#N)c(NC(=O)Cc3ccc(C(F)(F)F)cc3)c2)c2cnc(N)nc21]

To a solution of 2-amino-4-{2-amino-7-[2-(tert-butyl-dimethyl-silanyloxy)-1,1-dimethyl-ethyl]-7H-pyrrolo[2,3-d]pyrimidine-5-carbonyl}-benzonitrile (Preparation 12, 60 mg, 0.13 mmol), (4-trifluoromethyl-phenyl)-acetic acid (21 mg, 0.103 mmol) and Et₃N (0.062 mL, 0.451 mmol) in THF (2 mL), T3P (50% solution in EtOAc, 0.23 mL, 0.387 mmol) was added and the mixture was stirred at room temperature for 18 hours. The reaction was evaporated under reduced pressure, the residue partitioned between water and ethyl acetate, the organic extracts washed with saturated sodium bicarbonate solution, dried over sodium sulphate and evaporated in vacuo. The crude material was purified by silica gel column chromatography eluting with 0-5% MeOH in DCM to afford the title compound as solid (30 mg, 36%).

¹H NMR (400 MHz, DMSO-d₆): δ ppm−0.26 (s, 6H), 0.59 (s, 9H), 1.65 (s, 6H), 3.90 (s, 2H), 4.03 (s, 2H), 6.54 (s, 2H), 7.59-7.64 (m, 4H), 7.69 (d, 2H), 7.97-8.01 (m, 2H), 8.95 (s, 1H), 10.69 (s, 1H).

LCMS Rt=4.06 minutes MS m/z 651 [M+H]⁺

### Preparation 2

### N-{4-[(7-{[3-({[tert-Butyl(dimethyl)silyl]oxy}methyl)oxetan-3-yl]methyl}-7H-pyrrolo[2,3-d]pyrimidin-5-yl)carbonyl]pyridin-2-yl}-2-(4-chlorophenyl)acetamide

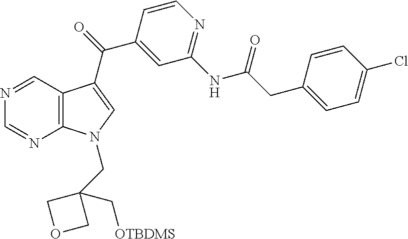 [SMILES: CC(C)(C)[Si](C)(C)OCC1(Cn2cc(C(=O)c3ccnc(NC(=O)Cc4ccc(Cl)cc4)c3)c3cncnc32)COC1]

The title compound was prepared according to the method described for Preparation using 2-(4-chlorophenyl)-N-[4-(7H-pyrrolo[2,3-d]pyrimidin-5-ylcarbonyl)pyridin-2-yl]acetamide (Preparation 109) and {[3-(bromomethyl)oxetan-3-yl]methoxy}(tert-butyl)dimethylsilane (Preparation 125) to afford the title compound as a white solid in 37% yield, 39 mg.

LCMS (system 2): Rt=1.58 minutes MS m/z 605, 607 [M+H]⁺

### Preparation 3

### 2-(3,5-Dimethyl-1H-pyrazol-1-yl)-N-[4-({7-[2-tert-butyl(dimethyl)silyloxy-1-(tert-butyl(dimethyl)silyloxymethyl)ethyl]-7H-pyrrolo[2,3-d]pyrimidin-5-yl}carbonyl)pyridin-2-yl]acetamide

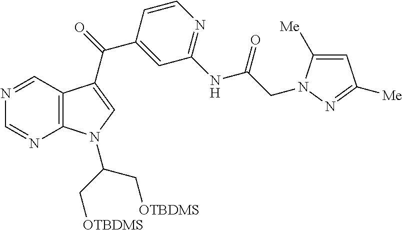 [SMILES: Cc1cc(C)n(CC(=O)Nc2cc(C(=O)c3cn(C(CO[Si](C)(C)C(C)(C)C)CO[Si](C)(C)C(C)(C)C)c4ncncc34)ccn2)n1]

The title compound was prepared according to the method described for Preparation using 2-(3,5-dimethyl-1H-pyrazol-1-yl)-N-[4-(7H-pyrrolo[2,3-d]pyrimidin-5-ylcarbonyl)pyridin-2-yl]acetamide (Preparation 108) and 2-tert-butyl(dimethyl)silyloxy-1-[tert-butyl(dimethyl)silyl]oxymethyl]ethyl trifluoromethanesulfonate (Preparation 123) to afford the title compound as a colourless gum in 36% yield, 245 mg.

¹H NMR (400 MHz, DMSO-d₆): δ ppm −0.15 (s, 6H), −0.12 (s, 6H), 0.64 (s, 18H), 2.05 (s, 3H), 2.18 (s, 3H), 3.95-4.08 (m, 4H), 4.96 (s, 2H), 5.04 (m, 1H), 5.80 (s, 1H), 7.38 (dd, 1H), 8.31-8.36 (m, 2H), 8.56 (dd, 1H), 9.00 (s, 1H), 9.47 (s, 1H), 11.05 (s, 1H).

### Preparation 4

### 2-(4-Cyanophenyl)-N-(4-{[7-(2-{[tert-butyl(dimethyl)silyl]oxy}-1,1-dimethylethyl)-7H-pyrrolo[2,3-d]pyrimidin-5-yl]carbonyl}pyridin-2-yl)acetamide

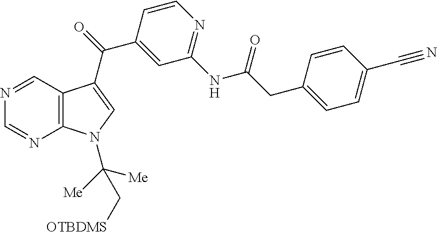 [SMILES: CC(C)(CO[Si](C)(C)C(C)(C)C)n1cc(C(=O)c2ccnc(NC(=O)Cc3ccc(C#N)cc3)c2)c2cncnc21]

4-Cyanophenylacetic acid (17 mg, 0.11 mmol) was added to (2-aminopyridin-4-yl)[7-(2-{[tert-butyl(dimethyl)silyl]oxy}-1,1-dimethylethyl)-7H-pyrrolo[2,3-d]pyrimidin-5-yl]methanone (Preparation 21, 35 mg, 0.80 mmol) and HATU (47 mg, 0.12 mmol) in pyridine (1 mL). The mixture was stirred at 50° C. for 16 hours. Saturated aqueous sodium bicarbonate (1 mL) was added then extracted with ethyl acetate (5 mL). The organic phase was dried over magnesium sulphate and evaporated in vacuo. The crude solid was purified by silica gel column chromatography eluting with a gradient of DCM:MeOH 100:0 to 95:5 to afford the title compound as a green oil (56%, 26 mg).

LCMS (system 2): Rt=1.64 minutes MS m/z 569 [M+H]⁺

### Preparation 5

### 2-(3,5-Dimethyl-1H-pyrazol-1-yl)-N-{4-[(7-{[2-(trimethylsilyl)ethoxy]methyl}-7H-pyrrolo[2,3-d]pyrimidin-5-yl)carbonyl]pyridin-2-yl}acetamide

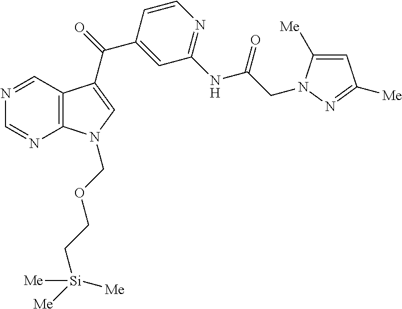 [SMILES: Cc1cc(C)n(CC(=O)Nc2cc(C(=O)c3cn(COCC[Si](C)(C)C)c4ncncc34)ccn2)n1]

The title compound was prepared according to the method described for Method 1 (Example 1) using (2-aminopyridin-4-yl)(7-{[2-(trimethylsilyl)ethoxy]methyl}-7H-pyrrolo[2,3-d]pyrimidin-5-yl)methanone (Preparation 15) and (3,5-dimethyl-pyrazol-1-yl)-acetic acid to afford the title compound as a white solid in 100% yield, 493 mg.

LCMS (system 1): Rt=3.25 minutes MS m/z 506 [M+H]⁺

### Preparation 6

### 2-(4-Chlorophenyl)-N-{4-[(7-{[2-(trimethylsilyl)ethoxy]methyl}-7H-pyrrolo[2,3-d]pyrimidin-5-yl)carbonyl]pyridin-2-yl}acetamide

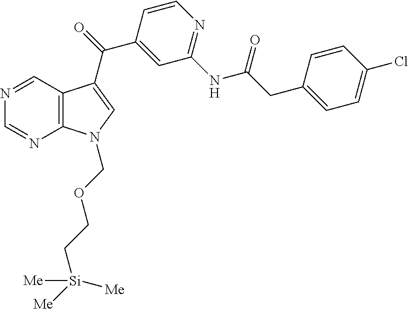 [SMILES: C[Si](C)(C)CCOCn1cc(C(=O)c2ccnc(NC(=O)Cc3ccc(Cl)cc3)c2)c2cncnc21]

The title compound was prepared according to the method described for Method 1 (Example 1) using (2-aminopyridin-4-yl)(7-{[2-(trimethylsilyl)ethoxy]methyl}-7H-pyrrolo[2,3-d]pyrimidin-5-yl)methanone (Preparation 15) and 4-chlorophenylacetic acid to afford the title compound as an off-white solid in 100% yield, 398 mg.

LCMS (system 2): Rt=1.74 minutes MS m/z 522, 524 [M+H]⁺

### Preparation 7

### (2-aminopyridin-4-yl)[7-(trans-4-{[tert-butyl(dimethyl)silyl]oxy}cyclohexyl)-7H-pyrrolo[2,3-d]pyrimidin-5-yl]methanone

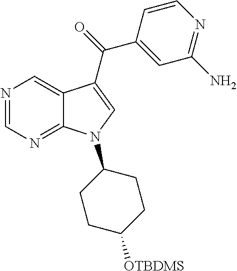 [SMILES: CC(C)(C)[Si](C)(C)O[C@H]1CC[C@H](n2cc(C(=O)c3ccnc(N)c3)c3cncnc32)CC1]

### Step 1

To a solution of 7-(trans-4-{[tert-butyl(dimethyl)silyl]oxy}cyclohexyl)-5-iodo-7H-pyrrolo[2,3-d]pyrimidine (Preparation 75, 1190 mg, 2.58 mmol) in THF (10 mL) was added ⁱPrMgCl (1.55 mL, 3.10 mmol, 2M in THF) dropwise at 0° C. After stirring for 1 hour at the same temperature, a solution of 2-[(diphenylmethylene)amino]-N-methoxy-N-methylisonicotinamide (Preparation 115, 1160 mg, 3.35 mmol) in THF (3 mL) was added slowly to the reaction mixture. The reaction was warmed to room temperature and stirred for 16 hours. The reaction was quenched by the addition of 10% aqueous NH₄Cl solution (50 mL) and extracted into EtOAc thrice (3×50 mL). The organic layer was collected, dried over sodium sulphate and concentrated in vacuo. The residue was purified using silica gel column chromatography eluting with a gradient of heptane:ethyl acetate 100:0 to 30:70 to afford the benzhydryl protected amino intermediate.

### Step 2

To this intermediate dissolved in THF (10 mL) was added 1N citric acid solution (5 mL) and the reaction stirred at room temperature for 2 hours. The reaction was basified with 1N aqueous NaOH solution (10 mL) and extracted into EtOAc thrice (3×20 mL). The organic layer was collected, dried over sodium sulphate and concentrated in vacuo. The residue was purified using silica gel column chromatography eluting with a gradient of heptane:ethyl acetate 50:50 to 0:100 to afford the title compound as a white solid (466 mg, 40%).

¹HNMR (400 MHz, DMSO-d₆): δ ppm 0.07 (s, 6H), 0.88 (s, 9H), 1.41-1.58 (m, 2H), 1.88-2.01 (m, 4H), 2.06-2.22 (m, 2H), 3.71-3.83 (m, 1H), 4.66-4.79 (m, 1H), 6.25 (br s, 2H), 6.72-6.76 (m, 1H), 6.79 (dd, 1H), 8.10 (dd, 1H), 8.40 (s, 1H), 8.98 (s, 1H), 9.43 (s, 1H).

LCMS Rt=2.35 minutes MS m/z 452 [M+H]⁺

### Preparation 8

### Racemic (2-aminopyridin-4-yl)[7-(2-hydroxy-1-methylethyl)-7H-pyrrolo[2,3-d]pyrimidin-5-yl]methanone

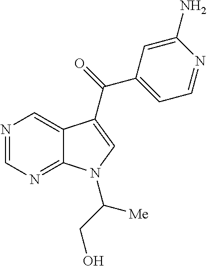 [SMILES: CC(CO)n1cc(C(=O)c2ccnc(N)c2)c2cncnc21]

The title compound was prepared according to Preparation 7 using 2-[(diphenylmethylene)amino]-N-methoxy-N-methylisonicotinamide (Preparation 115) and 7-(2-{[tert-butyl(dimethyl)silyl]oxy}-1-methylethyl)-5-iodo-7H-pyrrolo[2,3-d]pyrimidine (Preparation 62).

¹H NMR (400 MHz, DMSO-d₆): δ ppm 1.51 (d, 3H), 3.68-3.78 (m, 1H), 3.82-3.92 (m, 1H), 4.92-5.07 (m, 2H), 6.27 (br s, 2H), 6.73-6.77 (m, 1H), 6.80 (dd, 1H), 8.10 (dd, 1H), 8.36 (s, 1H), 8.97 (s, 1H), 9.42 (s, 1H).

LCMS Rt=1.34 minutes, MS m/z 298 [M+H]⁺

### Preparation 9

### (2-aminopyridin-4-yl)[7-(cis-4-{[tert-butyl(dimethyl)silyl]oxy}cyclohexyl)-7H-pyrrolo[2,3-d]pyrimidin-5-yl]methanone

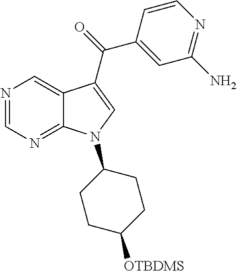 [SMILES: CC(C)(C)[Si](C)(C)O[C@H]1CC[C@@H](n2cc(C(=O)c3ccnc(N)c3)c3cncnc32)CC1]

Prepared according to the method described for the trans isomer (Preparation 7) using 7-(cis-4-{[tert-butyl(dimethyl)silyl]oxy}cyclohexyl)-5-iodo-7H-pyrrolo[2,3-d]pyrimidine (Preparation 77).

¹H NMR (400 MHz, DMSO-d₆): δppm 0.07 (s, 6H), 0.92 (s, 9H), 1.59-1.89 (m, 6H), 2.26-2.45 (m, 2H), 4.04-4.13 (m, 1H), 4.63-4.78 (m, 1H), 6.26 (br s, 2H), 6.74-6.82 (m, 2H), 8.05-8.11 (m, 1H), 8.15 (s, 1H), 8.95 (s, 1H), 9.41 (s, 1H).

LCMS Rt=2.39 minutes, MS m/z 452 [M+H]⁺

### Preparation 10

### 2-fluoro-4-[(7-isopropyl-7H-pyrrolo[2,3-d]pyrimidin-5-yl)carbonyl]benzonitrile

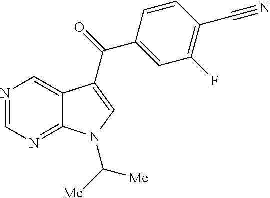 [SMILES: CC(C)n1cc(C(=O)c2ccc(C#N)c(F)c2)c2cncnc21]

The title compound was prepared according to Preparation 55 using 4-cyano-3-fluoro-N-methoxy-N-methyl-benzamide and 5-iodo-7-isopropyl-7H-pyrrolo[2,3-d]pyrimidine (Preparation 79).

¹H NMR (400 MHz, DMSO-d₆): δ ppm 1.54 (d, 6H), 5.07 (m, 1H), 7.82 (d, 1H), 7.95 (d, 1H), 8.16 (t, 1H), 8.49 (s, 1H), 9.00 (s, 1H), 9.45 (s, 1H).

### Preparation 11

### 2-amino-4-[(7-isopropyl-7H-pyrrolo[2,3-d]pyrimidin-5-yl)carbonyl]benzonitrile

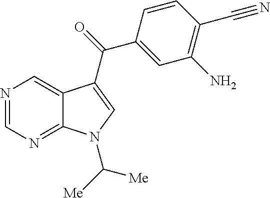 [SMILES: CC(C)n1cc(C(=O)c2ccc(C#N)c(N)c2)c2cncnc21]

The title compound was prepared according to Preparation 14 followed by Preparation 13 using 2-fluoro-4-[(7-isopropyl-7H-pyrrolo[2,3-d]pyrimidin-5-yl)carbonyl]benzonitrile (Preparation 10). The crude residue was purified using preparative TLC eluting with 7.5% MeOH in DCM.

¹H NMR (400 MHz, DMSO-d₆): δ ppm 1.55 (d, 6H), 5.08 (m, 1H), 6.36 (s, 2H), 6.98 (d, 1H), 7.20 (s, 1H), 7.58 (d, 1H), 8.38 (s, 1H), 8.90 (s, 1H), 9.40 (s, 1H).

### Preparation 12

### 2-Amino-4-{2-amino-7-[2-(tert-butyl-dimethyl-silanyloxy)-1,1-dimethyl-ethyl]-7H-pyrrolo[2,3-d]pyrimidine-5-carbonyl}-benzonitrile

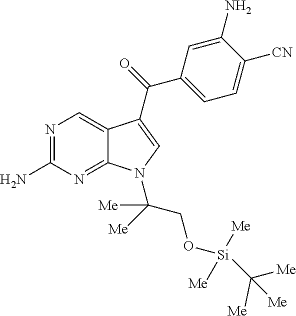 [SMILES: CC(C)(CO[Si](C)(C)C(C)(C)C)n1cc(C(=O)c2ccc(C#N)c(N)c2)c2cnc(N)nc21]

To a solution of 2-amino-4-[2-amino-7-(2-hydroxy-1,1-dimethyl-ethyl)-7H-pyrrolo[2,3-d]pyrimidine-5-carbonyl]-benzonitrile (Preparation 13, 230 mg, 0.66 mmol) in DCM (10 mL) were added 2,6-lutidine (0.23 mL, 1.98 mmol) and TBDMS triflate (0.16 mL, 0.66 mmol) at 0° C. and the resultant reaction mixture was allowed to stir at room temperature for 16 hours. The reaction mixture was diluted with EtOAc and the organic layer was washed with water, brine, dried over sodium sulfate and evaporated in vacuo. The crude material was purified by silica gel column chromatography eluting with 0-5% MeOH in DCM to afford the title compound as solid in 45% yield, 140 mg.

¹H NMR (400 MHz, DMSO-d₆): δ ppm −0.18 (s, 6H), 0.66 (s, 9H), 1.66 (s, 6H), 4.03 (s, 2H), 6.31 (br s, 2H), 6.52 (br s, 2H), 6.86 (d, 1H), 7.11 (s, 1H), 7.51 (d, 1H), 7.55 (s, 1H), 8.92 (s, 1H).

LCMS Rt=3.76 minutes MS m/z 465 [M+H]⁺

### Preparation 13

### 2-Amino-4-[2-amino-7-(2-hydroxy-1,1-dimethyl-ethyl)-7H-pyrrolo[2,3-d]pyrimidine-5-carbonyl]-benzonitrile

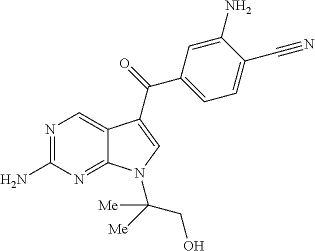 [SMILES: CC(C)(CO)n1cc(C(=O)c2ccc(C#N)c(N)c2)c2cnc(N)nc21]

To 4-[7-[1,1-Dimethyl-2-(tetrahydro-pyran-2-yloxy)-ethyl]-2-(4-methoxy-benzylamino)-7H-pyrrolo[2,3-d]pyrimidine-5-carbonyl]-2-(4-methoxy-benzylamino)-benzonitrile (Preparation 14, 600 mg, 0.89 mmol) was added TFA (10 mL) and the resultant mixture was stirred at room temperature for 16 hours. The reaction mixture was then evaporated to dryness, basified with saturated NaHCO₃ and extracted with ethyl acetate (2 times). The combined organic layers were washed brine, dried over sodium sulphate and evaporated in vacuo. The crude material was purified by silica gel column chromatography eluting with 2-3% MeOH in DCM to afford the title compound as a solid in 73% yield, 230 mg.

¹HNMR (400 MHz, DMSO-d₆): δ ppm 1.63 (s, 6H), 3.89 (d, 2H), 5.04 (t, 1H), 6.30 (s, 2H), 6.49 (s, 2H), 6.90 (d, 1H), 7.15 (s, 1H), 7.53 (d, 1H), 7.58 (s, 1H), 8.92 (s, 1H).

LCMS Rt=2.88 minutes MS m/z 351 [M+H]⁺

### Preparation 14

### 4-[7-[1,1-Dimethyl-2-(tetrahydro-pyran-2-yloxy)-ethyl]-2-(4-methoxy-benzylamino)-7H-pyrrolo[2,3-d]pyrimidine-5-carbonyl]-2-(4-methoxy-benzylamino)-benzonitrile

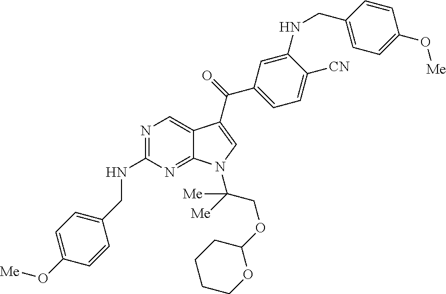 [SMILES: COc1ccc(CNc2ncc3c(C(=O)c4ccc(C#N)c(NCc5ccc(OC)cc5)c4)cn(C(C)(C)COC4CCCCO4)c3n2)cc1]

To a solution of 4-{2-chloro-7-[1,1-dimethyl-2-(tetrahydro-pyran-2-yloxy)-ethyl]-7H-pyrrolo[2,3-d]pyrimidine-5-carbonyl}-2-fluoro-benzonitrile (Preparation 47, 600 mg, 1.31 mmol) in DMSO (5 mL), 4-methoxy benzyl amine (500 μL, 3.93 mmol) was added and the resultant reaction mixture was allowed to stir at 100° C. for 16 hours. The reaction mixture was then diluted with EtOAc and washed with water (2-3 times), brine, dried over sodium sulphate and evaporated in vacuo. The crude material was purified by silica gel column chromatography eluting with a gradient of 50-60% EtOAc in Hexane to afford the title compound as gum in 68% yield, 600 mg.

LCMS Rt=3.80 minutes MS m/z 675 [M+H]⁺

### Preparation 15

### (2-Aminopyridin-4-yl)(7-{[2-(trimethylsilyl)ethoxy]methyl}-7H-pyrrolo[2,3-d]pyrimidin-5-yl)methanone

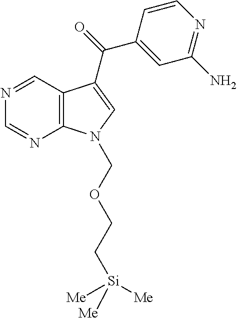 [SMILES: C[Si](C)(C)CCOCn1cc(C(=O)c2ccnc(N)c2)c2cncnc21]

The title compound was prepared according to the method described for Preparation 21 using {2-[(diphenylmethylene)amino]pyridin-4-yl}(7-{[2-(trimethylsilyl)ethoxy]methyl}-7H-pyrrolo[2,3-d]pyrimidin-5-yl)methanone (Preparation 37) to afford the title compound as a white solid in 85% yield, 373 mg.

¹H NMR (400 MHz, DMSO-d₆): δ ppm −0.10 (s, 9H), 0.84 (t, 2H), 3.60 (t, 2H), 5.71 (s, 2H), 6.28 (br s, 2H), 6.76 (m, 1H), 6.79 (dd, 1H), 8.11 (dd, 1H), 8.52 (s, 1H), 9.03 (s, 1H), 9.47 (s, 1H).

### Preparation 16

### {2-amino-7-[1,1-dimethyl-2-(tetrahydro-2H-pyran-2-yloxy)ethyl]-7H-pyrrolo[2,3-d]pyrimidin-5-yl}(4-aminopyridin-2-yl)methanone

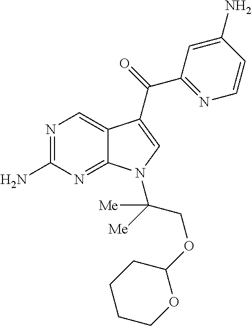 [SMILES: CC(C)(COC1CCCCO1)n1cc(C(=O)c2cc(N)ccn2)c2cnc(N)nc21]

To a solution of (4-bromopyridin-2-yl){2-chloro-7-[1,1-dimethyl-2-(tetrahydro-2H-pyran-2-yloxy)ethyl]-7H-pyrrolo[2,3-d]pyrimidin-5-yl}methanone (Preparation 48, 375 mg, 0.756 mmol) in NMP (2 mL) in a sealed tube was added formaldehyde (32 mg, 0.220 mmol) and 880 ammonia (10 mL) and the reaction heated to 140° C. for 16 hours. The reaction was cooled and diluted with 20% IPA in DCM. The aqueous layer was collected and extracted four times with 20% IPA in DCM. The organic layers were combined, dried over sodium sulphate and concentrated in vacuo. The residue was purified using silica gel column chromatography eluting with 2-5% MeOH in DCM to afford the title compound.

¹HNMR (400 MHz, DMSO-d₆): δ ppm 1.11-1.53 (m, 6H), 1.71 (s, 3H), 1.74 (s, 3H), 3.37 (m, 1H), 3.54 (m, 1H), 3.88 (d, 1H), 4.13 (d, 1H), 4.53 (m, 1H), 6.30 (br s, 2H), 6.44 (br s, 2H), 6.63 (d, 1H), 7.17 (s, 1H), 8.11 (d, 1H), 8.80 (s, 1H), 9.04 (s, 1H).

### Preparation 17

### (4-Aminopyridin-2-yl)(7-isopropyl-7H-pyrrolo[2,3-d]pyrimidin-5-yl)methanone

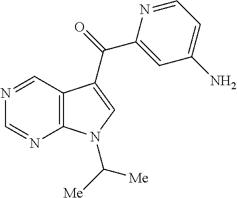 [SMILES: CC(C)n1cc(C(=O)c2cc(N)ccn2)c2cncnc21]

Copper sulphate pentahydrate (165 mg, 0.66 mmol) was added to (4-bromopyridin-2-yl)(7-isopropyl-7H-pyrrolo[2,3-d]pyrimidin-5-yl)methanone (Preparation 57, 760 mg, 2.20 mmol) and concentrated aqueous ammonia solution (50 mL). The mixture was heated in a sealed vessel at 140° C. for 17 hours. The reaction mixture was evaporated in vacuo and the residue was stirred in 2M HCl at room temperature for 17 hours. The reaction mixture was basified to pH 9 using saturated aqueous sodium carbonate then extracted with DCM (3×20 mL). The combined organic extracts were dried over magnesium sulfate and evaporated in vacuo to afford the title compound as a white solid in 47% yield, 288 mg.

¹H NMR (400 MHz, CDCl₃): δ ppm 1.25 (d, 6H), 4.50 (br s, 2H), 5.16 (m, 1H), 6.70 (m, 1H), 7.46 (s, 1H), 8.34 (d, 1H), 8.97 (s, 1H), 9.06 (s, 1H), 9.71 (s, 1H).

LCMS (system 2): Rt=0.29 minutes MS m/z 282 [M+H]⁺

### Preparation 18

### (2-Aminopyridin-4-yl)(7-cyclopropyl-7H-pyrrolo[2,3-d]pyrimidin-5-yl)methanone

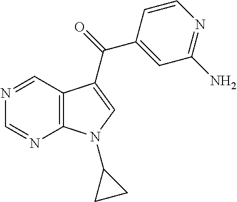 [SMILES: Nc1cc(C(=O)c2cn(C3CC3)c3ncncc23)ccn1]

To a solution of (7-cyclopropyl-7H-pyrrolo[2,3-d]pyrimidin-5-yl){2-[(diphenylmethylene)amino]pyridin-4-yl}methanone (Preparation 40, 40 mg, 0.090 mmol) in THF (3 mL) was added 1N HCl (1 mL) and the reaction stirred at room temperature for 1.5 hours. The reaction was diluted with water (10 mL) and extracted with EtOAc (2×15 mL). The aqueous layer was basified to pH 10 using sodium carbonate and further extracted with EtOAc (2×15 mL). The organic layers were combined, dried over MgSO₄ and concentrated in vacuo to afford the title compound as a white powder (32 mg, >100%).

LCMS (System 2): Rₜ=0.58 minutes MS m/z 280 [M+H]⁺

### Preparation 19

### (4-aminopyridin-2-yl)[7-(3-methyloxetan-3-yl)-7H-pyrrolo[2,3-d]pyrimidin-5-yl]methanone

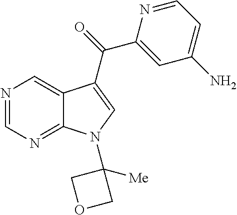 [SMILES: CC1(n2cc(C(=O)c3cc(N)ccn3)c3cncnc32)COC1]

The title compound was prepared according to the method described for Preparation 7 using 5-iodo-7-(3-methyloxetan-3-yl)-7H-pyrrolo[2,3-d]pyrimidine (Preparation 61) and 4-[(diphenylmethylene)amino]-N-methoxy-N-methylpyridine-2-carboxamide (Preparation 111).

1H NMR (400 MHz, DMSO-d₆): δ ppm 1.91 (s, 3H), 4.79 (d, 2H), 5.21 (d, 2H), 6.38 (br s, 2H), 6.69 (dd, 1H), 7.25 (d, 1H), 8.21 (d, 1H), 8.92 (s, 1H), 9.07 (s, 1H), 9.56 (s, 1H).

LCMS Rt=1.73 minutes MS m/z 310 [M+H]⁺

### Preparation 20

### (2-Amino-7-isopropyl-7H-pyrrolo[2,3-d]pyrimidin-5-yl)(4-aminopyridin-2-yl)methanone

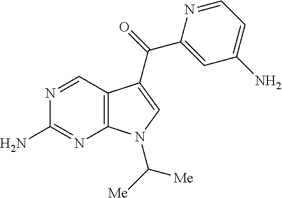 [SMILES: CC(C)n1cc(C(=O)c2cc(N)ccn2)c2cnc(N)nc21]

The title compound was prepared according to the method described for Preparation 17 using Cu^{l}O and (2-chloro-7-isopropyl-7H-pyrrolo[2,3-d]pyrimidin-5-yl)(4-chloropyridin-2-yl)methanone (Preparation 46) at 80° C. for 17 hours to afford the title compound as a yellow solid in 40% yield, 600 mg.

¹H NMR (400 MHz, DMSO-d₆): δ ppm 1.47 (d, 6H), 4.83 (m, 1H), 6.29 (s, 2H), 6.49 (s, 2H), 6.64 (dd, 1H), 7.16 (d, 1H), 8.15 (d, 1H), 8.69 (d, 1H), 9.01 (s, 1H).

LCMS (System 10) Rₜ=2.37 minutes MS m/z 297 [M+H]⁺

### Preparation 21

### (2-Aminopyridin-4-yl)[7-(2-{[tert-butyl(dimethyl)silyl]oxy}-1,1-dimethylethyl)-7H-pyrrolo[2,3-d]pyrimidin-5-yl]methanone

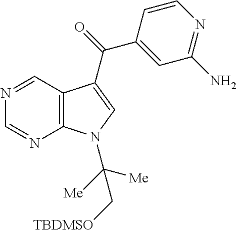 [SMILES: CC(C)(CO[Si](C)(C)C(C)(C)C)n1cc(C(=O)c2ccnc(N)c2)c2cncnc21]

Aqueous citric acid (0.5 mL, 1.0 M) was added to [7-(2-{[tert-butyl(dimethyl)silyl]oxy}-1,1-dimethylethyl)-7H-pyrrolo[2,3-d]pyrimidin-5-yl]{2-[(diphenylmethylene)amino]pyridin-4-yl}methanone (Preparation 39, 80 mg, 0.63 mmol) in THF (1 mL) and the mixture was stirred at room temperature for 16 hours. Water (2 mL) and ethyl acetate (4 mL) were added to the mixture and the aqueous phase was basified using aqueous sodium hydroxide (1 mL, 1.0 M). The aqueous layer was extracted with EtOAc (3×50 mL), the combined organic phases were dried over sodium sulphate and evaporated in vacuo. The crude solid was purified by silica gel column chromatography eluting with a gradient of heptane:EtOAc 100:0 to 50:50 to afford the title compound as a colourless oil in 59% yield, 35 mg.

¹H NMR (400 MHz, CDCl₃): δ ppm 0.00 (s, 6H), 0.90 (s, 9H), 1.99 (s, 6H), 4.29 (s, 2H), 7.04 (s, 1H), 7.12 (m, 1H), 8.07 (s, 1H), 8.38 (m, 1H), 9.17 (s, 1H), 9.81 (s, 1H).

### Preparation 22

### (4-aminopyridin-2-yl){7-[(1R)-1-methyl-2-(tetrahydro-2H-pyran-2-yloxy)ethyl]-7H-pyrrolo[2,3-d]pyrimidin-5-yl}methanone

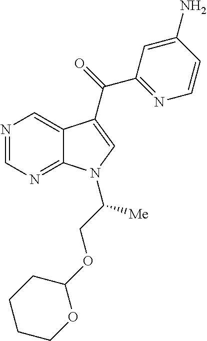 [SMILES: C[C@H](COC1CCCCO1)n1cc(C(=O)c2cc(N)ccn2)c2cncnc21]

The title compound was prepared according to Preparation 21 using {4-[(diphenylmethylene)amino]pyridin-2-yl}{7-[(1R)-1-methyl-2-(tetrahydro-2H-pyran-2-yloxy)ethyl]-7H-pyrrolo[2,3-d]pyrimidin-5-yl}methanone (Preparation 42).

LCMS Rt=0.67 minutes MS m/z 382 [M+H]⁺

### Preparation 23

### (4-aminopyridin-2-yl){7-[(1S)-1-methyl-2-(tetrahydro-2H-pyran-2-yloxy)ethyl]-7H-pyrrolo[2,3-d]pyrimidin-5-yl}methanone

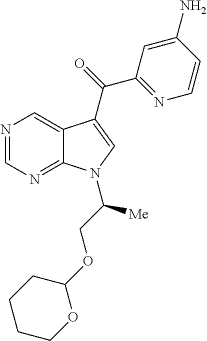 [SMILES: C[C@@H](COC1CCCCO1)n1cc(C(=O)c2cc(N)ccn2)c2cncnc21]

The title compound was prepared according to Preparation 22 using the (S) enantiomer (Preparation 43).

### Preparation 24

### (2-Aminopyridin-4-yl)(7-isopropyl-7H-pyrrolo[2,3-d]pyrimidin-5-yl)methanone

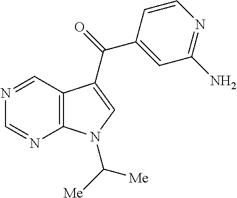 [SMILES: CC(C)n1cc(C(=O)c2ccnc(N)c2)c2cncnc21]

Copper sulphate pentahydrate (225 mg, 0.90 mmol) was added to (2-bromopyridin-4-yl)(7-isopropyl-7H-pyrrolo[2,3-d]pyrimidin-5-yl)methanone (Preparation 54, 1040 mg, 3.0 mmol) and concentrated aqueous ammonia solution (30 mL). The reaction was heated in a sealed vessel at 140° C. for 16 hours before cooling and extracting with EtOAc three times (3×30 mL). The organic layers were collected and concentrated in vacuo. The residue was diluted with 1N HCl (10 mL) and stirred for 10 minutes followed by the addition of 1N aqueous NaOH solution until pH=14. The mixture was extracted with EtOAc three times (3×30 mL), the organic layers combined, dried over MgSO₄ and concentrated in vacuo to obtain the crude residue. The residue was purified using silica gel column chromatography eluting with 0-20% MeOH in EtOAc to afford the title compound as a white solid (535 mg, 60%).

¹H NMR (400 MHz, DMSO-d₆): δ ppm 1.56 (d, 6H), 5.08 (m, 1H), 6.25 (br s, 2H), 6.76 (md, 1H), 6.81 (dd, 1H), 8.10 (dd, 1H), 8.41 (s, 1H), 8.98 (s, 1H), 9.42 (s, 1H).

LCMS (system 1): Rt=1.78 minutes MS m/z 282 [M+H]⁺

### Preparation 25

### (2-amino-6-methylpyridin-4-yl)(7-isopropyl-7H-pyrrolo[2,3-d]pyrimidin-5-yl)methanone

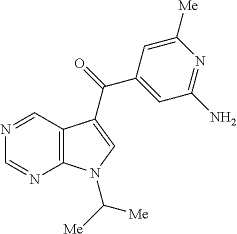 [SMILES: Cc1cc(C(=O)c2cn(C(C)C)c3ncncc23)cc(N)n1]

The title compound was prepared according to Preparation 17 using (2-chloro-6-methylpyridin-4-yl)(7-isopropyl-7H-pyrrolo[2,3-d]pyrimidin-5-yl)methanone (Preparation 52) at 140° C. for 48 hours. The residue was purified using silica gel column chromatography eluting with EtOAc.

¹H NMR (400 MHz, CDCl₃): δ ppm 1.55 (s, 6H), 2.45 (s, 3H), 4.60 (br s, 2H), 5.10 (m, 1H), 6.60 (s, 1H), 6.80 (m, 1H), 7.80 (m, 1H), 9.0 (s, 1H), 9.55 (s, 1H).

### Preparation 26

### (2-aminopyridin-4-yl)(2-hydroxy-7-isopropyl-7H-pyrrolo[2,3-d]pyrimidin-5-yl)methanone

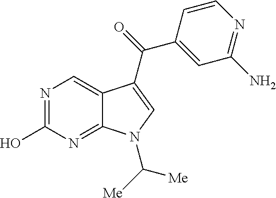 [SMILES: CC(C)n1cc(C(=O)c2ccnc(N)c2)c2cnc(O)nc21]

A solution of (2-chloro-7-isopropyl-7H-pyrrolo[2,3-d]pyrimidin-5-yl){2-[(diphenylmethylene)amino]pyridin-4-yl}methanone (Preparation 36, 60 mg, 0.125 mmol) in 6N HCl (5 mL) was heated to 80° C. for 36 hours. The reaction was cooled and concentrated in vacuo. The residue was partitioned between saturated aqueous NaHCO₃ solution and EtOAc, the organic layer was collected, washed with brine, dried over MgSO₄ and concentrated in vacuo. The residue was purified using preparative TLC eluting with 10% MeOH in DCM to afford the title compound (13 mg, 20%).

¹H NMR (400 MHz, DMSO-d₆): δ ppm 1.42 (d, 6H), 4.74 (t, 1H), 6.22 (s, 2H), 6.69 (s, 1H), 6.73 (d, 1H), 8.03 (s, 1H), 8.06 (d, 1H), 8.45 (s, 1H), 12.0 (s, 1H).

### Preparation 27

### (2-Aminopyridin-4-yl)[7-(2,2-dimethyl-1,3-dioxan-5-yl)-7H-pyrrolo[2,3-d]pyrimidin-5-yl]methanone

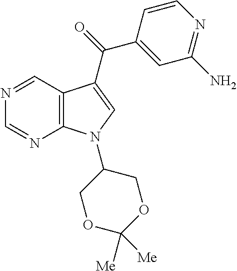 [SMILES: CC1(C)OCC(n2cc(C(=O)c3ccnc(N)c3)c3cncnc32)CO1]

2,2-Dimethoxypropane (0.34 mL, 2.80 mmol) was added to 2-(3,5-dimethyl-1H-pyrazol-1-yl)-N-[4-({7-[2-hydroxy-1-(hydroxymethyl)ethyl]-7H-pyrrolo[2,3-d]pyrimidin-5-yl}carbonyl)pyridin-2-yl]acetamide (Example 112, 63 mg, 0.14 mmol) and 4-toluenesulphonic acid monohydrate (53 mg, 0.28 mmol) in acetone (1 mL) and DMF (2 mL). The mixture was heated to 50° C. for 17 hours then evaporated in vacuo. The crude residue was dissolved in MeOH (5 mL) and aqueous sodium hydroxide (5 mL, 1.0 M). The mixture was heated to 50° C. for 17 hours before water (10 mL) was added. The mixture was extracted with EtOAc (3×20 mL) and the combined organic phases were dried over sodium sulfate and evaporated in vacuo to afford the title compound as an off-white solid in 85% yield, 42 mg.

LCMS (system 2): Rt=0.61 minutes MS m/z 354 [M+H]⁺

### Preparation 28

### (2-aminopyridin-4-yl)(7-tert-butyl-7H-pyrrolo[2,3-d]pyrimidin-5-yl)methanone

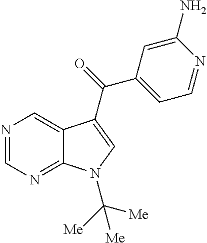 [SMILES: CC(C)(C)n1cc(C(=O)c2ccnc(N)c2)c2cncnc21]

Aqueous HCl solution (1.0 M, 5 mL) was added to (7-tert-butyl-7H-pyrrolo[2,3-d]pyrimidin-5-yl){2-[(diphenylmethylene)amino]pyridin-4-yl}methanone (Preparation 38, 672 mg, 1.46 mmol) in THF (5 mL) and the mixture was stirred at room temperature for 30 minutes. Water (20 mL) and ethyl acetate (20 mL) were added to the mixture and the aqueous phase was basified using aqueous sodium hydroxide (1.0 M, 20 mL). The aqueous layer was extracted with EtOAc (3×50 mL), the combined organic phases were dried over sodium sulphate and evaporated in vacuo. The crude solid was purified by crystallization using heptane:EtOAc to afford the title compound as a white solid in 88% yield, 381 mg.

¹H NMR (400 MHz, DMSO-d₆): δ ppm 1.80 (s, 9H), 6.25 (br s, 2H), 6.80 (m, 1H), 6.85 (dd, 1H), 8.08-8.13 (m, 2H), 9.00 (s, 1H), 9.45 (s, 1H).

LCMS (system 1): Rt=1.64 minutes MS m/z 296 [M+H]⁺

### Preparation 29

### (4-aminopyridin-2-yl)(7-tert-butyl-7H-pyrrolo[2,3-d]pyrimidin-5-yl)methanone

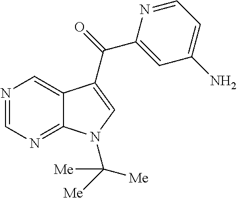 [SMILES: CC(C)(C)n1cc(C(=O)c2cc(N)ccn2)c2cncnc21]

A solution of [4-(benzhydrylidene-amino)-pyridin-2-yl]-(7-tert-butyl-7H-pyrrolo[2,3-d]pyrimidin-5-yl)-methanone (Preparation 41, 350 mg, 0.76 mmol) in THF (10 mL) and citric acid (1 N, 10 mL) was stirred at room temperature for 6 hours. The solvent was evaporated and the residue was partitioned between saturated NaHCO₃ solution and EtOAc. The organic layer was separated, dried over sodium sulfate and evaporated in vacuo. The crude material was purified by silica gel column chromatography eluting with 0-5% MeOH in DCM to afford the title compound as brown solid in 49% yield, 110 mg.

¹H NMR (400 MHz, DMSO-d₆): δ ppm 1.81 (s, 9H), 6.38 (br s, 2H), 6.68 (dd, 1H), 7.25 (d, 1H), 8.20 (d, 1H), 8.95 (s, 1H), 9.22 (s, 1H), 9.57 (s, 1H).

LCMS: Rt=2.93 minutes MS m/z 296 [M+H]⁺

### Preparation 30

### (6-aminopyrimidin-4-yl)(7-isopropyl-7H-pyrrolo[2,3-d]pyrimidin-5-yl)methanone

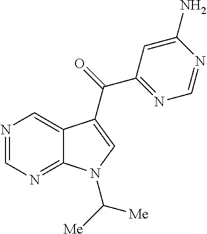 [SMILES: CC(C)n1cc(C(=O)c2cc(N)ncn2)c2cncnc21]

A mixture of (6-chloropyrimidin-4-yl)(7-isopropyl-7H-pyrrolo[2,3-d]pyrimidin-5-yl)methanone (Preparation 49, 530 mg, 1.76 mmol) and CuSO₄.5H₂O (132 mg, 0.527 mmol) in ammonium hydroxide (15 mL) was heated to 140° C. in an autoclave for 18 hours. The reaction was cooled, and partitioned between water and DCM. The organic layer was collected, washed with brine, dried over MgSO₄ and concentrated in vacuo to afford the title compound as a brown solid (496 mg, 84%).

¹H NMR (400 MHz, DMSO-d₆): δ ppm 1.55 (d, 6H), 4.97-5.14 (m, 1H), 6.99 (s, 1H), 7.28 (br s, 2H), 8.56 (s, 1H), 8.95 (s, 1H), 9.17 (s, 1H), 9.51 (s, 1H).

LCMS Rt=0.85 minutes MS m/z 283 [M+H]⁺

### Preparation 31

### (2-Amino-7-isopropyl-7H-pyrrolo[2,3-d]pyrimidin-5-yl)(2-aminopyridin-4-yl)methanone

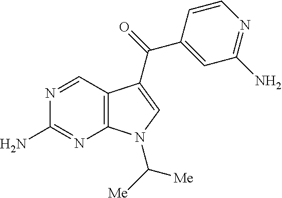 [SMILES: CC(C)n1cc(C(=O)c2ccnc(N)c2)c2cnc(N)nc21]

Copper sulphate pentahydrate (79 mg, 0.32 mmol) was added to (2-bromopyridin-4-yl)(2-chloro-7-isopropyl-7H-pyrrolo[2,3-d]pyrimidin-5-yl)methanone (Preparation 56, 400 mg, 1.055 mmol) and concentrated ammonia solution (10 mL). The reaction was heated in a sealed vessel at 130° C. for 32 hours before cooling and concentrating in vacuo to low volume. The residue was extracted into 10% iPrOH in DCM, the organic layer collected, dried over Na₂SO₄ and concentrated in vacuo. The residue was purified using silica gel column chromatography eluting with 5% MeOH in DCM to afford the title compound (180 mg, 58%).

¹H NMR (400 MHz, DMSO-d₆): δ ppm 1.46 (d, 6H), 4.81-4.87 (m, 1H), 6.20 (s, 2H), 6.60 (s, 2H), 6.70 (s, 1H), 6.73 (d, 1H), 7.86 (s, 1H), 8.05 (d, 1H), 8.90 (s, 1H).

LCMS (System 10): Rt=2.29 minutes MS m/z 297 [M+H]⁺

### Preparation 32

### (4-aminopyridin-2-yl){7-[2-{[tert-butyl(dimethyl)silyl]oxy}-1-({[tert-butyl(dimethyl)silyl]oxy}methyl)ethyl]-7H-pyrrolo[2,3-d]pyrimidin-5-yl}methanone

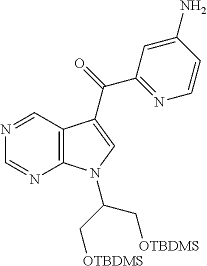 [SMILES: CC(C)(C)[Si](C)(C)OCC(CO[Si](C)(C)C(C)(C)C)n1cc(C(=O)c2cc(N)ccn2)c2cncnc21]

The title compound was prepared according to Preparation 7 using 7-[2-{[tert-butyl(dimethyl)silyl]oxy}-1-({[tert-butyl(dimethyl)silyl]oxy}methyl)ethyl]-5-iodo-7H-pyrrolo[2,3-d]pyrimidine (Preparation 59) and 4-[(diphenylmethylene)amino]-N-methoxy-N-methylpyridine-2-carboxamide (Preparation 111).

¹HNMR (400 MHz, DMSO-d₆): δ ppm −0.08 (s, 6H), −0.06 (s, 6H), 0.73 (s, 18H), 3.98-4.17 (m, 4H), 5.03-5.12 (m, 1H), 6.34 (br s, 2H), 6.68 (dd, 1H), 7.24 (d, 1H), 8.13 (d, 1H), 8.94 (s, 1H), 9.30 (s, 1H), 9.55 (s, 1H).

LCMS Rt=2.59 minutes, MS m/z 542 [M+H]⁺

### Preparation 33

### (4-aminopyridin-2-yl){7-[2-{[tert-butyl(dimethyl)silyl]oxy}-1-({[tert-butyl(dimethyl)silyl]oxy}methyl)-1-methylethyl]-7H-pyrrolo[2,3-d]pyrimidin-5-yl}methanone

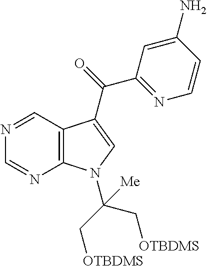 [SMILES: CC(CO[Si](C)(C)C(C)(C)C)(CO[Si](C)(C)C(C)(C)C)n1cc(C(=O)c2cc(N)ccn2)c2cncnc21]

The title compound was prepared according to Preparation 7 using 7-(2-{[tert-butyl(dimethyl)silyl]oxy}-1-{[(tert-butylsilyl)oxy]methyl}-1-methylethyl)-5-iodo-7H-pyrrolo[2,3-d]pyrimidine (Preparation 60).

¹HNMR (400 MHz, DMSO-d₆): δ ppm −0.13 (s, 6H), −0.11 (s, 6H), 0.71 (s, 18H), 1.77 (s, 3H), 4.09 (d, 2H), 4.35 (d, 2H), 6.35 (br s, 2H), 6.67 (dd, 1H), 7.23 (d, 1H), 8.13 (d, 1H), 8.91 (s, 1H), 9.28 (s, 1H), 9.57 (s, 1H).

LCMS Rt=6.48 minutes, MS m/z 556 [M+H]⁺

### Preparation 34

### (2-Aminopyridin-4-yl){7-[2-(tetrahydro-2H-pyran-2-yloxy)ethyl]-7H-pyrrolo[2,3-d]pyrimidin-5-yl}methanone

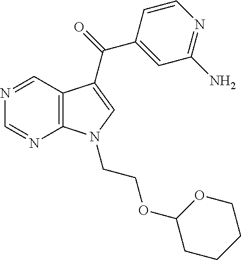 [SMILES: Nc1cc(C(=O)c2cn(CCOC3CCCCO3)c3ncncc23)ccn1]

The title compound was prepared according to the method described for Preparation 17 using (2-bromopyridin-4-yl){7-[2-(tetrahydro-2H-pyran-2-yloxy)ethyl]-7H-pyrrolo[2,3-d]pyrimidin-5-yl}methanone (Preparation 55) to afford the title compound as a yellow oil in 45% yield, 2.3 g.

¹H NMR (400 MHz, DMSO-d₆): δ ppm 1.31-1.39 (m, 4H), 1.49-1.51 (m, 2H), 3.31-3.37 (m, 2H), 3.79 (m, 1H), 3.95 (m, 1H), 4.51-4.57 (m, 3H), 6.24 (s, 2H), 6.73 (s, 1H), 6.77 (d, 1H), 8.09 (d, 1H), 8.37 (s, 1H), 8.99 (s, 1H), 9.44 (s, 1H).

LCMS (System 9): Rt=2.73 minutes MS m/z 368 [M+H]⁺

### Preparation 35

### (4-Aminopyridin-2-yl)[7-(2-{[tert-butyl(dimethyl)silyl]oxy}-1,1-dimethylethyl)-7H-pyrrolo[2,3-d]pyrimidin-5-yl]methanone

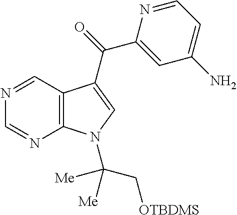 [SMILES: CC(C)(CO[Si](C)(C)C(C)(C)C)n1cc(C(=O)c2cc(N)ccn2)c2cncnc21]

The title compound was prepared according to the method described for Preparation using [7-(2-{[tert-butyl(dimethyl)silyl]oxy}-1,1-dimethylethyl)-7H-pyrrolo[2,3-d]pyrimidin-5-yl]{4-[(diphenylmethylene)amino]pyridin-2-yl}methanone (Preparation 44) to afford the title compound as a white solid in 66% yield, 930 mg.

¹H NMR (400 MHz, CDCl₃): δ ppm 0.17 (s, 6H), 0.72 (s, 9H), 1.84 (s, 6H), 4.14 (s, 2H), 4.32 (br s, 2H), 6.99 (dd, 1H), 7.42 (d, 1H), 8.34 (d, 1H), 8.93 (s, 1H), 9.15 (s, 1H), 9.74 (s, 1H).

### Preparation 36

### (2-chloro-7-isopropyl-7H-pyrrolo[2,3-d]pyrimidin-5-yl){2-[(diphenylmethylene)amino]pyridin-4-yl}methanone

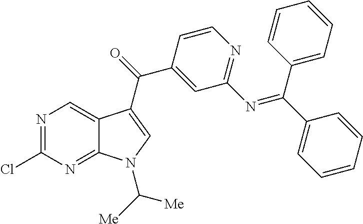 [SMILES: CC(C)n1cc(C(=O)c2ccnc(N=C(c3ccccc3)c3ccccc3)c2)c2cnc(Cl)nc21]

The title compound was prepared according to Preparation 55 using 2-[(diphenylmethylene)amino]-N-methoxy-N-methylisonicotinamide (Preparation 115) and 2-chloro-5-iodo-7-isopropyl-7H-pyrrolo[2,3-d]pyrimidine (Preparation 78).

¹H NMR (400 MHz, DMSO-d₆): δ ppm 1.53 (d, 6H), 4.97 (m, 1H), 7.09 (s, 1H), 7.20 (m, 2H), 7.28 (m, 1H), 7.34 (m, 2H), 7.51-7.59 (m, 4H), 7.71 (d, 2H), 8.27 (s, 1H), 8.45 (d, 1H), 9.24 (s, 1H).

LCMS Rt=3.83 minutes MS m/z 481 [M+H]⁺

### Preparation 37

### {2-[(Diphenylmethylene)amino]pyridin-4-yl}(7-{[2-(trimethylsilyl)ethoxy]methyl}-7H-pyrrolo[2,3-d]pyrimidin-5-yl)methanone

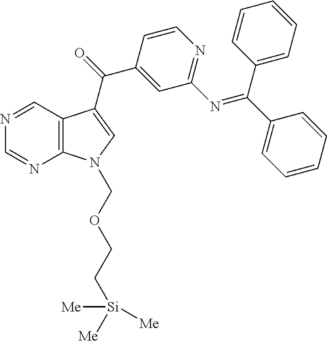 [SMILES: C[Si](C)(C)CCOCn1cc(C(=O)c2ccnc(N=C(c3ccccc3)c3ccccc3)c2)c2cncnc21]

The title compound was prepared according to the method described for Preparation using 5-iodo-7-{[2-(trimethylsilyl)ethoxy]methyl}-7H-pyrrolo[2,3-d]pyrimidine (Preparation 66) and 2-[(diphenylmethylene)amino]-N-methoxy-N-methylisonicotinamide (Preparation 115) to afford the title compound as a yellow solid in 66% yield, 632 mg.

LCMS (system 1): Rt=3.81 minutes MS m/z 534 [M+H]⁺

### Preparation 38

### (7-tert-Butyl-7H-pyrrolo[2,3-d]pyrimidin-5-yl){2-[(diphenylmethylene)amino]pyridin-4-yl}methanone

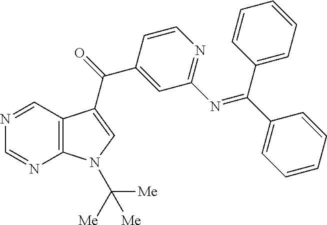 [SMILES: CC(C)(C)n1cc(C(=O)c2ccnc(N=C(c3ccccc3)c3ccccc3)c2)c2cncnc21]

The title compound was prepared according to the method described for Preparation 7 using 7-tert-butyl-5-iodo-7H-pyrrolo[2,3-d]pyrimidine (Preparation 80) and 2-[(diphenylmethylene)amino]-N-methoxy-N-methylisonicotinamide (Preparation 115) to afford the title compound as a yellow solid in 73% yield, 672 mg.

¹H NMR (400 MHz, DMSO-d₆): δ ppm 1.79 (s, 9H), 7.17-7.27 (m, 3H), 7.29 (dd, 1H), 7.31-7.39 (m, 3H), 7.46-7.64 (m, 3H), 7.65-7.78 (m, 2H), 7.94 (s, 1H), 8.42 (dd, 1H), 9.00 (s, 1H), 9.41 (s, 1H). LCMS (system 1): Rt=3.57 minutes MS m/z 460 [M+H]⁺

### Preparation 39

### [7-(2-{[tert-Butyl(dimethyl)silyl]oxy}-1,1-dimethylethyl)-7H-pyrrolo[2,3-d]pyrimidin-5-yl]{2-[(diphenylmethylene)amino]pyridin-4-yl}methanone

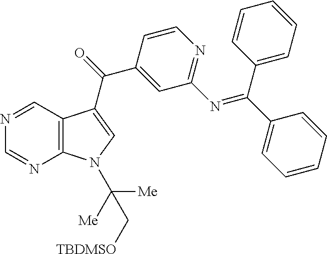 [SMILES: CC(C)(CO[Si](C)(C)C(C)(C)C)n1cc(C(=O)c2ccnc(N=C(c3ccccc3)c3ccccc3)c2)c2cncnc21]

The title compound was prepared according to the method described for Preparation 7 using 7-(2-{[tert-butyl(dimethyl)silyl]oxy}-1,1-dimethylethyl)-5-iodo-7H-pyrrolo[2,3-d]pyrimidine (Preparation 82) and 2-[(diphenylmethylene)amino]-N-methoxy-N-methylisonicotinamide (Preparation 115) to afford the title compound as a yellow oil in 22% yield, 80 mg.

¹H NMR (400 MHz, CDCl₃): δ ppm 0.00 (s, 6H), 0.89 (s, 9H), 1.99 (s, 6H), 4.30 (s, 2H), 7.17 (s, 1H), 7.38 (m, 1H), 7.57-7.65 (m, 10H), 7.97 (s, 1H), 8.68 (m, 1H), 9.18 (s, 1H), 9.77 (s, 1H).

### Preparation 40

### (7-Cyclopropyl-7H-pyrrolo[2,3-d]pyrimidin-5-yl){2-[(diphenylmethylene)amino]pyridin-4-yl}methanone

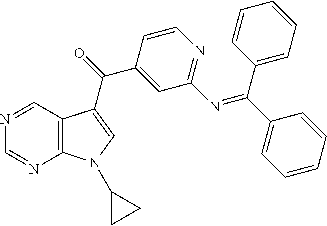 [SMILES: O=C(c1ccnc(N=C(c2ccccc2)c2ccccc2)c1)c1cn(C2CC2)c2ncncc12]

The title compound was prepared according to the method described for Preparation 7 using 7-cyclopropyl-5-iodo-7H-pyrrolo[2,3-d]pyrimidine (Preparation 76) and 2-[(diphenylmethylene)amino]-N-methoxy-N-methylisonicotinamide (Preparation 115) to afford the title compound as a brown oil (60 mg, 7%).

LCMS (System 2): Rt=1.42 minutes MS m/z 444 [M+H]⁺

### Preparation 41

### [4-(Benzhydrylidene-amino)-pyridin-2-yl]-(7-tert-butyl-7H-pyrrolo[2,3-d]pyrimidin-5-yl)-methanone

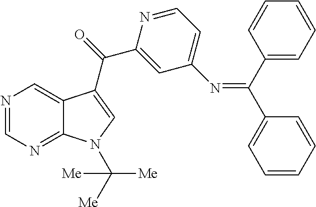 [SMILES: CC(C)(C)n1cc(C(=O)c2cc(N=C(c3ccccc3)c3ccccc3)ccn2)c2cncnc21]

A mixture of (7-tert-butyl-7H-pyrrolo[2,3-d]pyrimidin-5-yl)-(4-chloro-pyridin-2-yl)-methanone (Preparation 36, 500 mg, 1.59 mmol), benzophenone imine (0.32 mL, 1.90 mmol) and cesium carbonate (2.6 gm, 7.92 mmol) in toluene (10 mL) was degassed with argon followed by the addition of Pd(OAc)₂ (36 mg, 0.16 mmol) and BINAP (99 mg, 0.16 mmol) and heated at 110° C. for 6 hours. The reaction was filtered on a bed of celite and the filtrate was evaporated under reduced pressure. The crude material was purified by silica gel column chromatography eluting with 20-25% EtOAc in hexane to afford the title compound as gum in 62% yield, 450 mg.

¹H NMR (400 MHz, DMSO-d₆): δ ppm 1.83 (s, 9H), 7.05 (dd, 1H), 7.26-7.36 (m, 5H), 7.42 (d, 1H), 7.51-7.70 (m, 5H), 8.52 (d, 1H), 8.96 (s, 1H), 9.18 (s, 1H), 9.55 (s, 1H).

LCMS: Rt=4.06 minutes MS m/z 460 [M+H]⁺

### Preparation 42

### {4-[(diphenylmethylene)amino]pyridin-2-yl}{7-[(1R)-1-methyl-2-(tetrahydro-2H-pyran-2-yloxy)ethyl]-7H-pyrrolo[2,3-d]pyrimidin-5-yl}methanone

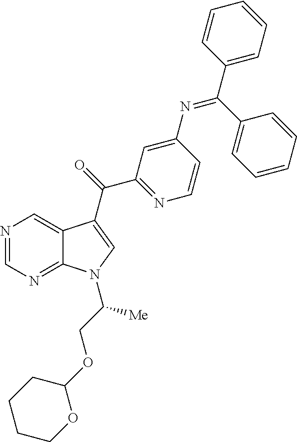 [SMILES: C[C@H](COC1CCCCO1)n1cc(C(=O)c2cc(N=C(c3ccccc3)c3ccccc3)ccn2)c2cncnc21]

The title compound was prepared according to Preparation 41 using (4-chloropyridin-2-yl){7-[(1R)-1-methyl-2-(tetrahydro-2H-pyran-2-yloxy)ethyl]-7H-pyrrolo[2,3-d]pyrimidin-5-yl}methanone (Preparation 50). The crude residue was used directly in the next reaction.

### Preparation 43

### {4-[(diphenylmethylene)amino]pyridin-2-yl}{7-[(1S)-1-methyl-2-(tetrahydro-2H-pyran-2-yloxy)ethyl]-7H-pyrrolo[2,3-d]pyrimidin-5-yl}methanone

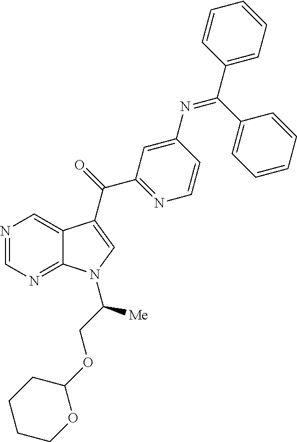 [SMILES: C[C@@H](COC1CCCCO1)n1cc(C(=O)c2cc(N=C(c3ccccc3)c3ccccc3)ccn2)c2cncnc21]

The title compound was prepared according to Preparation 41 using the (S) enantiomer (Preparation 51).

### Preparation 44

### [7-(2-{[tert-Butyl(dimethyl)silyl]oxy}-1,1-dimethylethyl)-7H-pyrrolo[2,3-d]pyrimidin-5-yl]{4-[(diphenylmethylene)amino]pyridin-2-yl}methanone

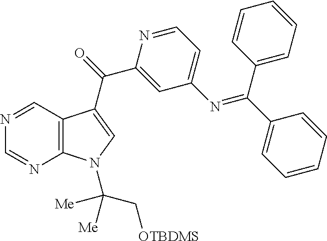 [SMILES: CC(C)(CO[Si](C)(C)C(C)(C)C)n1cc(C(=O)c2cc(N=C(c3ccccc3)c3ccccc3)ccn2)c2cncnc21]

The title compound was prepared according to the method described for Preparation using NaOtBu and [7-(2-{[tert-butyl(dimethyl)silyl]oxy}-1,1-dimethylethyl)-7H-pyrrolo[2,3-d]pyrimidin-5-yl](4-chloropyridin-2-yl)methanone (Preparation 58) to afford the title compound as a green solid in 60% yield, 1.94 g.

¹H NMR (400 MHz, CDCl₃): δ ppm 0.00 (s, 6H), 0.89 (s, 9H), 2.01 (s, 6H), 4.31 (s, 2H), 6.99 (dd, 1H), 7.33-7.72 (m, 10H), 7.74 (d, 1H), 8.61 (d, 1H), 9.11 (s, 1H), 9.33 (s, 1H), 9.90 (s, 1H).

### Preparation 45

### (2-chloro-6-methoxypyridin-4-yl)(7-isopropyl-7H-pyrrolo[2,3-d]pyrimidin-5-yl)methanone

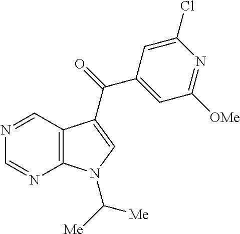 [SMILES: COc1cc(C(=O)c2cn(C(C)C)c3ncncc23)cc(Cl)n1]

Prepared according to Preparation 7 Step 1 using 5-iodo-7-isopropyl-7H-pyrrolo[2,3-d]pyrimidine (Preparation 79) and 2-chloro-N,6-dimethoxy-N-methyl-4-pyridylcarboxamide (Preparation 114).

¹H NMR (400 MHz, CDCl₃): δ ppm 1.60 (s, 6H), 4.00 (s, 3H), 5.20 (m, 1H), 6.90 (s, 1H), 7.20 (s, 1H), 7.75 (s, 1H), 9.00 (s, 1H), 9.55 (s, 1H).

### Preparation 46

### (2-Chloro-7-isopropyl-7H-pyrrolo[2,3-d]pyrimidin-5-yl)(4-chloropyridin-2-yl)methanone

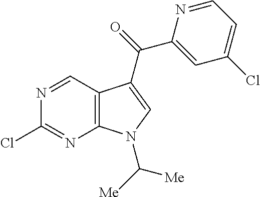 [SMILES: CC(C)n1cc(C(=O)c2cc(Cl)ccn2)c2cnc(Cl)nc21]

The title compound was prepared according to the method described for Preparation 48 using 2-chloro-5-iodo-7-isopropyl-7H-pyrrolo[2,3-d]pyrimidine (Preparation 78) and 4-chloro-N-methoxy-N-methylpyridine-2-carboxamide afford the title compound as a brown solid in 37% yield, 2.10 g.

¹H NMR (400 MHz, CDCl₃): δ 1.61 (d, 6H), 5.15 (m, 1H), 7.51 (dd, 1H), 8.20 (d, 1H), 8.64 (m, 1H), 9.06 (d, 1H), 9.56 (s, 1H).

LCMS (System 10) Rt=4.08 minutes MS m/z 335 [M+H]⁺

### Preparation 47

### 4-{2-Chloro-7-[1,1-dimethyl-2-(tetrahydro-pyran-2-yloxy)-ethyl]-7H-pyrrolo[2,3-d]pyrimidine-5-carbonyl}-2-fluoro-benzonitrile

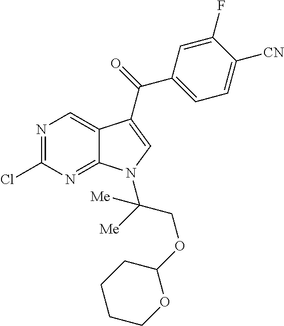 [SMILES: CC(C)(COC1CCCCO1)n1cc(C(=O)c2ccc(C#N)c(F)c2)c2cnc(Cl)nc21]

To a solution of 2-chloro-7-[1,1-dimethyl-2-(tetrahydro-pyran-2-yloxy)-ethyl]-5-iodo-7H-pyrrolo[2,3-d]pyrimidine (Preparation 65, 1.6 gm, 3.67 mmol) in ether (25 mL), nBuLi (2.3M in hexane, 1.59 mL, 3.67 mmol) was added at −78° C. After the addition, 4-cyano-3-fluoro-N-methoxy-N-methyl-benzamide (Preparation 116, 688 mg, 3.31 mmol) was added immediately as a solution in ether (5 mL) and the resultant mixture was allowed to stir at −78° C. for 1 hour and at room temperature for 1 hour. The reaction mixture was quenched with saturated NH₄Cl solution and diluted with EtOAc and water. The layers were separated and aqueous layer was further extracted with EtOAc. The combined organic layers were washed with brine, dried over sodium sulphate and evaporated in vacuo. The crude material was purified by silica gel column chromatography eluting with a gradient of 30-40% EtOAc in Hexane to afford the title compound as solid in 36% yield, 600 mg.

¹H NMR (400 MHz, DMSO-d₆): δ ppm 1.24-1.50 (m, 6H), 1.75 (d, 6H), 3.35-3.37 (m, 2H), 3.84 (d, 1H), 4.19 (d, 1H), 4.50 (s, 1H), 7.81 (d, 1H), 7.94 (d, 1H), 8.16 (t, 1H), 8.22 (s, 1H), 9.36 (s, 1H).

LCMS Rt=3.90 minutes MS m/z 457 [M+H]⁺

### Preparation 48

### (4-bromopyridin-2-yl){2-chloro-7-[1,1-dimethyl-2-(tetrahydro-2H-pyran-2-yloxy)ethyl]-7H-pyrrolo[2,3-d]pyrimidin-5-yl}methanone

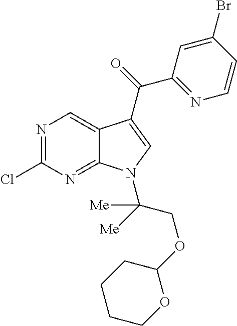 [SMILES: CC(C)(COC1CCCCO1)n1cc(C(=O)c2cc(Br)ccn2)c2cnc(Cl)nc21]

To a solution of 2-chloro-7-[1,1-dimethyl-2-(tetrahydro-pyran-2-yloxy)-ethyl]-5-iodo-7H-pyrrolo[2,3-d]pyrimidine (Preparation 65, 900 mg, 2.07 mmol) in ether (10 mL) was added nBuLi (2.08M in hexane, 0.984 mL, 2.07 mmol) at −78° C. After the addition, 4-bromo-pyridine-2-carboxylic acid methoxy-methyl-amide (Preparation 113, 453 mg, 1.85 mmol) was added immediately as a solution in ether (5 mL) and the resultant mixture was allowed to stir at −78° C. for 1 hour and at room temperature for 1 hour. The reaction mixture was quenched with saturated NH₄Cl solution and diluted with EtOAc and water. The layers were separated and aqueous layer was further extracted with EtOAc. The combined organic layers were washed with brine, dried over sodium sulphate and evaporated in vacuo. The crude residue was purified using silica gel column chromatography eluting with 25% EtOAc in hexane to afford the title compound (375 mg).

¹HNMR (400 MHz, CDCl₃): δ ppm 1.41-1.63 (m, 5H), 1.78-1.80 (m, 1H), 1.84 (s, 3H), 1.87 (s, 3H), 3.40-3.43 (m, 1H), 3.56-3.61 (m, 1H), 3.98 (d, 1H), 4.20 (d, 1H), 4.53 (m, 1H), 7.66 (dd, 1H), 8.36 (d, 1H), 8.51 (d, 1H), 9.17 (s, 1H), 9.57 (s, 1H).

### Preparation 49

### (6-chloropyrimidin-4-yl)(7-isopropyl-7H-pyrrolo[2,3-d]pyrimidin-5-yl)methanone

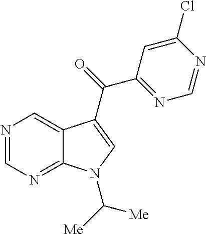 [SMILES: CC(C)n1cc(C(=O)c2cc(Cl)ncn2)c2cncnc21]

To a solution of 5-iodo-7-isopropyl-7H-pyrrolo[2,3-d]pyrimidine (Preparation 79, 2 g, 7.0 mmol) in THF (35 mL) was added ⁱPrMgCl (4.18 mL, 8.36 mmol, 2 M solution in diethyl ether) at 0° C. The reaction was stirred at this temperature for 1 hour before the addition of a solution of methyl 6-chloropyrimidine-4-carboxylate (1.51 g, 8.75 mmol) in THF (5 mL). The reaction was warmed to room temperature and stirred for 18 hours. The reaction was quenched by the addition of saturated aqueous ammonium chloride solution and extracted into DCM thrice. The organic layers were combined, washed with brine and concentrated in vacuo. The residue was purified using silica gel column chromatography eluting with 10-60% EtOAc in heptane to afford a yellow solid that was triturated with heptane to afford the title compound as a white solid (436 mg, 19%).

¹H NMR (400 MHz, CDCl₃): δ ppm 1.66 (d, 6H), 5.20 (m, 1H), 8.16 (d, 1H), 9.02 (s, 1H), 9.13 (s, 1H), 9.20 (d, 1H), 9.72 (s, 1H).

LCMS Rt=1.23 minutes MS m/z 302 [M+H]⁺

### Preparation 50

### (4-chloropyridin-2-yl){7-[(1R)-1-methyl-2-(tetrahydro-2H-pyran-2-yloxy)ethyl]-7H-pyrrolo[2,3-d]pyrimidin-5-yl}methanone

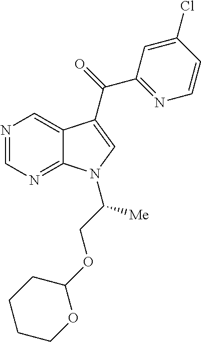 [SMILES: C[C@H](COC1CCCCO1)n1cc(C(=O)c2cc(Cl)ccn2)c2cncnc21]

The title compound was prepared according to Preparation 49 using 5-iodo-7-[(1R)-1-methyl-2-(tetrahydro-2H-pyran-2-yloxy)ethyl]-7H-pyrrolo[2,3-d]pyrimidine (Preparation 63) and 4-chloro-pyridine-2-carboxylic acid methoxy-methyl-amide (Preparation 112). The residue was taken directly on to the next step.

### Preparation 51

### (4-chloropyridin-2-yl){7-[(1S)-1-methyl-2-(tetrahydro-2H-pyran-2-yloxy)ethyl]-7H-pyrrolo[2,3-d]pyrimidin-5-yl}methanone

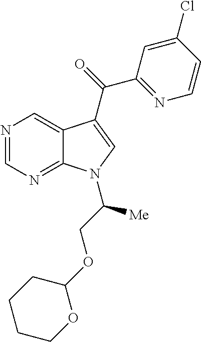 [SMILES: C[C@@H](COC1CCCCO1)n1cc(C(=O)c2cc(Cl)ccn2)c2cncnc21]

The title compound was prepared according to Preparation 49 using the (S) enantiomer (Preparation 64).

### Preparation 52

### (2-chloro-6-methylpyridin-4-yl)(7-isopropyl-7H-pyrrolo[2,3-d]pyrimidin-5-yl)methanone

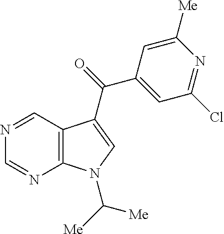 [SMILES: Cc1cc(C(=O)c2cn(C(C)C)c3ncncc23)cc(Cl)n1]

The title compound was prepared according to Preparation 49 using 5-iodo-7-isopropyl-7H-pyrrolo[2,3-d]pyrimidine (Preparation 79) and 2-chloro-N-methoxy-N,6-dimethylisonicotinamide as a white solid (4.75 g, 49%).

LCMS (System 1): Rt=2.96 minutes MS m/z 315 [M+H]⁺

### Preparation 53

### (7-tert-Butyl-7H-pyrrolo[2,3-d]pyrimidin-5-yl)-(4-chloro-pyridin-2-yl)-methanone

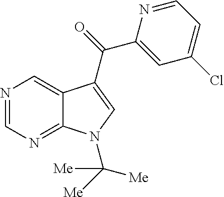 [SMILES: CC(C)(C)n1cc(C(=O)c2cc(Cl)ccn2)c2cncnc21]

7-tert-Butyl-5-iodo-7H-pyrrolo[2,3-d]pyrimidine (Preparation 80, 3.6 gm, 11.96 mmol) was taken in dry ether (75 mL), cooled to −78° C. and nBuLi (1.9 M, 6.92 mL, 13.15 mmol) was added dropwise. The reaction mixture was stirred at this temperature for 45 minutes and a solution of 4-chloro-pyridine-2-carboxylic acid methoxy-methyl-amide (Preparation 112, 2.4 gm, 11.96 mmol) in dry ether (25 mL) was added into the reaction mixture and stirring was continued for another 15 minutes. The reaction was quenched with saturated NH₄Cl solution and diluted with EtOAc. The organic layer was separated and washed with water, brine, dried over sodium sulfate and evaporated in vacuo. The crude material was purified by silica gel column chromatography eluting with 10-15% EtOAc in hexane to afford the title compound as light yellow solid in 54% yield, 2.6 g.

¹H NMR (400 MHz, DMSO-d₆): δ ppm 1.82 (s, 9H), 7.85-7.87 (m, 1H), 8.12 (s, 1H), 8.80 (d, 1H), 8.99 (s, 1H), 9.11 (s, 1H), 9.59 (s, 1H).

LCMS Rt=3.65 minutes MS m/z 315 [M+H]⁺

### Preparation 54

### (2-Bromopyridin-4-yl)(7-isopropyl-7H-pyrrolo[2,3-d]pyrimidin-5-yl)methanone

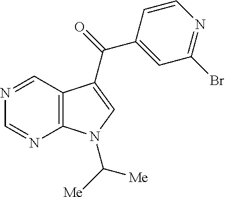 [SMILES: CC(C)n1cc(C(=O)c2ccnc(Br)c2)c2cncnc21]

To a solution of 5-iodo-7-isopropyl-7H-pyrrolo[2,3-d]pyrimidine (Preparation 79, 1440 mg, 5.0 mmol) in THF (25 mL) was added ⁱPrMgCl (2.75 mL, 5.5 mmol, 2 M solution in THF) at 0° C. The reaction was stirred at this temperature for 30 minutes before the addition of a solution of 2-bromo-N-methoxy-N-methylisonicotinamide (1.47 g, 6.0 mmol) in THF (1 mL). The reaction was warmed to room temperature and stirred for 18 hours. The reaction was quenched by the addition of saturated aqueous ammonium chloride solution (100 mL) and extracted into EtOAc three times (3×100 ml). The organic layers were combined, washed with brine, dried over MgSO₄ and concentrated in vacuo. EtOAc was added to the crude residue to form a solution, followed by heptanes to effect precipitation. The resulting solid was filtered to afford the title compound as a white solid (1.10 g, 64%).

¹H NMR (400 MHz, DMSO-d₆): δ ppm 1.55 (d, 6H), 5.08 (m, 1H), 7.79 (dd, 1H), 7.94 (m, 1H), 8.53 (s, 1H), 8.63 (dd, 1H), 9.01 (s, 1H), 9.45 (s, 1H).

### Preparation 55

### (2-Bromopyridin-4-yl){7-[2-(tetrahydro-2H-pyran-2-yloxy)ethyl]-7H-pyrrolo[2,3-d]pyrimidin-5-yl}methanone

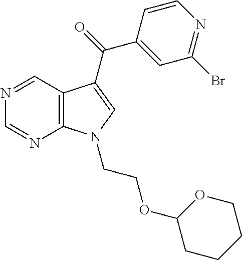 [SMILES: O=C(c1ccnc(Br)c1)c1cn(CCOC2CCCCO2)c2ncncc12]

n-Butyllithium (21.3 mL, 46.9 mmol, 2.2M in hexane) was added to 5-iodo-7-[2-(tetrahydro-2H-pyran-2-yloxy)ethyl]-7H-pyrrolo[2,3-d]pyrimidine (Preparation 81, 17.5 g, 46.91 mmol) in anhydrous diethyl ether (150 mL) under nitrogen at −78° C. and the mixture was stirred at −78° C. for 20 minutes. 2-Bromo-N-methoxy-N-methyl-isonicotinamide (9.7 g, 39.87 mmol in 80 mL anhydrous diethyl ether) was added and the mixture was stirred for 90 minutes at −78° C. then 90 minutes at room temperature. Cold aqueous saturated NH₄Cl (100 mL) was added to the mixture and extracted with ethyl acetate (2×100 mL). The combined organic phases were washed with water (100 mL), brine (50 mL), dried over sodium sulphate and evaporated in vacuo. The residue was purified by silica gel column chromatography on silica gel eluting with a gradient of hexane:EtOAc 50:50 to 20:80 to afford the title compound as a white solid in 51% yield, 10 g.

¹H NMR (400 MHz, DMSO-d₆): δ ppm 1.3-1.6 (m, 4H), 1.69-1.71 (m, 2H), 3.42 (m, 1H), 3.60 (s, 1H), 3.79 (m, 1H), 4.09 (m, 1H), 4.48-4.62 (m, 3H), 7.60 (dd, 1H), 7.82 (s, 1H), 7.93 (s, 1H), 8.56 (d, 1H), 9.02 (s, 1H), 9.62 (s, 1H).

LCMS (System 9): Rt=3.34 minutes MS m/z 431 [M+H]⁺

### Preparation 56

### (2-bromopyridin-4-yl)(2-chloro-7-isopropyl-7H-pyrrolo[2,3-d]pyrimidin-5-yl)methanone

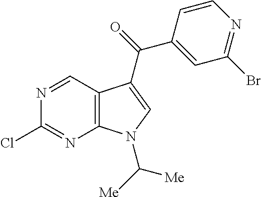 [SMILES: CC(C)n1cc(C(=O)c2ccnc(Br)c2)c2cnc(Cl)nc21]

n-Butyllithium (1.41 mL, 3.10 mmol, 2.2M in hexane) was added to 2-chloro-5-iodo-7-isopropyl-7H-pyrrolo[2,3-d]pyrimidine (Preparation 78, 1 g, 3.10 mmol) in anhydrous diethyl ether (25 mL) under nitrogen at −78° C. and the mixture was stirred at −78° C. for 30 minutes. 2-Bromo-N-methoxy-N-methyl-isonicotinamide (758 mg, 3.10 mmol in 10 mL anhydrous diethyl ether) was added and the mixture was stirred warming to room temperature for 3 hours. Cold aqueous saturated NH₄Cl (10 mL) was added to the mixture and extracted with ethyl acetate (2×10 mL). The combined organic phases were washed with water (10 mL), brine (5 mL), dried over sodium sulphate and evaporated in vacuo. The residue was triturated with hexane to afford the title compound (400 mg, 34%).

¹H NMR (400 MHz, DMSO-d₆): δ ppm 1.52 (s, 6H), 5.01 (m, 1H), 7.78 (dd, 1H), 7.93 (s, 1H), 8.56 (s, 1H), 8.63 (d, 1H), 9.32 (s, 1H).

### Preparation 57

### (4-bromopyridin-2-yl)(7-isopropyl-7H-pyrrolo[2,3-d]pyrimidin-5-yl)methanone

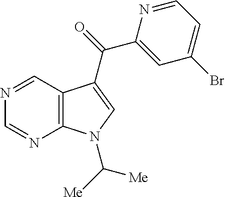 [SMILES: CC(C)n1cc(C(=O)c2cc(Br)ccn2)c2cncnc21]

n-Butyllithium (15.4 mL, 3.10 mmol, 2.2M in hexane) was added to 5-iodo-7-isopropyl-7H-pyrrolo[2,3-d]pyrimidine (Preparation 79, 7 g, 24.29 mmol) in anhydrous diethyl ether (100 mL) under nitrogen at −78° C. and the mixture was stirred at −78° C. for 30 minutes. 4-bromo-N-methoxy-N-methylpyridine-2-carboxamide (Preparation 113, 5.99 g, 24.47 mmol in 50 mL anhydrous diethyl ether) was added and the mixture was stirred warming to room temperature for 3 hours. Cold aqueous saturated NH₄Cl (10 mL) was added to the mixture and extracted with ethyl acetate (2×10 mL). The combined organic phases were washed with water (10 mL), brine (5 mL), dried over sodium sulphate and evaporated in vacuo. The residue was purified using silica gel column chromatography eluting with 15-30% EtOAc in Hexanes followed by trituration with pentane to afford the title compound (2.25 g, 26.7%).

¹H NMR (400 MHz, DMSO-d₆): δ ppm 1.58 (s, 6H), 5.10 (m, 1H), 8.00 (dd, 1H), 8.23 (d, 1H), 8.72 (d, 1H), 8.98 (s, 1H), 9.14 (s, 1H), 9.54 (s, 1H).

LCMS Rt=3.44 minutes MS m/z 345 [M+H]⁺

### Preparation 58

### [7-(2-{[tert-Butyl(dimethyl)silyl]oxy}-1,1-dimethylethyl)-7H-pyrrolo[2,3-d]pyrimidin-5-yl](4-chloropyridin-2-yl)methanone

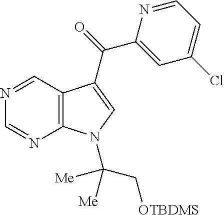 [SMILES: CC(C)(CO[Si](C)(C)C(C)(C)C)n1cc(C(=O)c2cc(Cl)ccn2)c2cncnc21]

The title compound was prepared according to the method described for Preparation 7 using 7-(2-{[tert-butyl(dimethyl)silyl]oxy}-1,1-dimethylethyl)-5-iodo-7H-pyrrolo[2,3-d]pyrimidine (Preparation 67) and 4-chloro-N-methoxy-N-methylpyridine-2-carboxamide to afford the title compound as a yellow solid in 70% yield, 3.04 g.

¹H NMR (400 MHz, CDCl₃): δ ppm 0.18 (s, 6H), 0.71 (s, 9H), 1.86 (s, 6H), 4.14 (s, 2H), 7.52 (dd, 1H), 8.23 (d, 1H), 8.63 (d, 1H), 8.79 (s, 1H), 8.99 (s, 1H), 9.23 (s, 1H).

### Preparation 59

### 7-[2-{[tert-butyl(dimethyl)silyl]oxy}-1-({[tert-butyl(dimethyl)silyl]oxy}methyl)ethyl]-5-iodo-7H-pyrrolo[2,3-d]pyrimidine

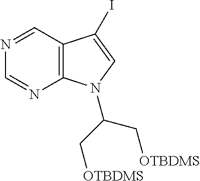 [SMILES: CC(C)(C)[Si](C)(C)OCC(CO[Si](C)(C)C(C)(C)C)n1cc(I)c2cncnc21]

The title compound was prepared according to Preparation 66 or Preparation 78 using 5-iodo-7H-pyrrolo[2,3-d]pyrimidine and 2-tert-butyl(dimethyl)silyloxy-1-[tert-butyl(dimethyl)silyl]oxymethyl]ethyl trifluoromethanesulfonate (Preparation 123).

¹HNMR (400 MHz, DMSO-d₆): δ ppm −0.13 (s, 6H), −0.07 (s, 6H), 0.71 (s, 18H), 3.96-4.04 (m, 4H), 4.90-5.01 (m, 1H), 7.91 (s, 1H), 8.71 (s, 1H), 8.82 (s, 1H).

### Preparation 60

### 7-(2-{[tert-butyl(dimethyl)silyl]oxy}-1-{[(tert-butylsilyl)oxy]methyl}-1-methylethyl)-5-iodo-7H-pyrrolo[2,3-d]pyrimidine

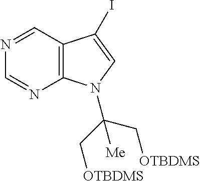 [SMILES: CC(CO[Si](C)(C)C(C)(C)C)(CO[Si](C)(C)C(C)(C)C)n1cc(I)c2cncnc21]

The title compound was prepared according to Preparation 80 using 7-(2-{[tert-butyl(dimethyl)silyl]oxy}-1-{[(tert-butylsilyl)oxy]methyl}-1-methylethyl)-7H-pyrrolo[2,3-d]pyrimidine (Preparation 105).

¹HNMR (400 MHz, CDCl₃): δ ppm −0.101 (d, 12H), 0.79 (s, 18H), 1.75 (s, 3H), 4.07 (d, 2H), 4.27 (d, 2H), 7.49 (s, 1H), 8.70 (s, 1H), 8.82 (s, 1H).

### Preparation 61

### 5-iodo-7-(3-methyloxetan-3-yl)-7H-pyrrolo[2,3-d]pyrimidine

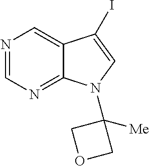 [SMILES: CC1(n2cc(I)c3cncnc32)COC1]

The title compound was prepared according to Preparation 80 using 7-(3-methyloxetan-3-yl)-7H-pyrrolo[2,3-d]pyrimidine (Preparation 102) for 16 hours. The reaction was poured into water (50 mL) and extracted into EtOAc thrice. The combined organic layers were washed with saturated aqueous Na₂S₂O₃, brine, dried over MgSO₄ and concentrated in vacuo. The residue was purified using silica gel column chromatography eluting with 20-30% EtOAc in heptane to afford the title compound as a white solid.

¹HNMR (400 MHz, DMSO-d₆): δ ppm 1.80 (s, 3H), 4.71 (d, 2H), 5.18 (d, 2H), 7.94 (s, 1H), 8.76 (s, 1H), 8.82 (s, 1H).

LCMS Rt=2.19 minutes, MS m/z 316 [M+H]⁺

### Preparation 62

### Racemic 7-(2-{[tert-butyl(dimethyl)silyl]oxy}-1-methylethyl)-5-iodo-7H-pyrrolo[2,3-d]pyrimidine

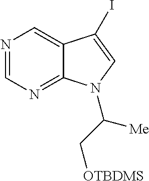 [SMILES: CC(CO[Si](C)(C)C(C)(C)C)n1cc(I)c2cncnc21]

The title compound was prepared according to Preparation 80 using 7-(2-{[tert-butyl(dimethyl)silyl]oxy}-1-methylethyl)-7H-pyrrolo[2,3-d]pyrimidine (Preparation 101). The residue was purified using silica gel column chromatography eluting with 0-60% EtOAc in heptanes.

¹H NMR (400 MHz, DMSO-d₆): δ ppm −0.24 (s, 3H), −0.16 (s, 3H), 0.64 (s, 9H), 1.48 (d, 3H), 3.80-3.91 (m, 2H), 4.93-5.04 (m, 1H), 7.96 (s, 1H), 8.70 (s, 1H), 8.81 (s, 1H).

LCMS Rt=4.03 minutes MS m/z 418 [M+H]⁺

### Preparation 63

### 5-iodo-7-[(1R)-1-methyl-2-(tetrahydro-2H-pyran-2-yloxy)ethyl]-7H-pyrrolo[2,3-d]pyrimidine

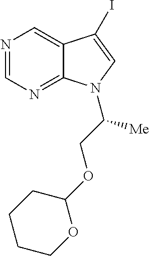 [SMILES: C[C@H](COC1CCCCO1)n1cc(I)c2cncnc21]

The title compound was prepared according to Preparation 80 using 7-[(1R)-1-methyl-2-(tetrahydro-2H-pyran-2-yloxy)ethyl]-7H-pyrrolo[2,3-d]pyrimidine (Preparation 99).

¹H NMR (400 MHz, CDCl₃): δ ppm 1.60-2.80 (m, 8H), 3.40-3.60 (m, 2H), 3.65-3.75 (m, 1H), 3.90-4.00 (m, 1H), 4.55-4.60 (m, 1H), 5.20-5.35 (m, 1H), 7.55 (s, 1H), 8.75 (s, 1H), 8.95 (s, 1H).

### Preparation 64

### 5-iodo-7-[(1S)-1-methyl-2-(tetrahydro-2H-pyran-2-yloxy)ethyl]-7H-pyrrolo[2,3-d]pyrimidine

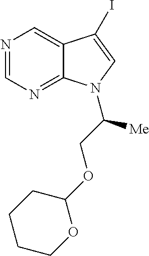 [SMILES: C[C@@H](COC1CCCCO1)n1cc(I)c2cncnc21]

The title compound was prepared according to Preparation 80 using the (S) enantiomer (Preparation 100).

### Preparation 65

### 2-Chloro-7-[1,1-dimethyl-2-(tetrahydro-2H-pyran-2-yloxy)ethyl]-5-iodo-7H-pyrrolo[2,3-d]pyrimidine

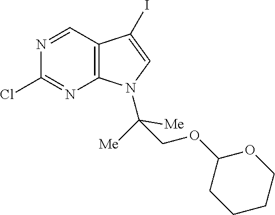 [SMILES: CC(C)(COC1CCCCO1)n1cc(I)c2cnc(Cl)nc21]

2,3-Dihydropyran (8.32 mL, 91.16 mmol) was added to 2-(2-chloro-5-iodo-7H-pyrrolo[2,3-d]pyrimidin-7-yl)-2-methylpropan-1-ol (Preparation 68, 8 g, 22.79 mmol) and pyridinium toluene-4-sulphonate (432 mg, 2.27 mmol) in THF (100 mL). The reaction was stirred at room temperature for 16 hours. The reaction mixture was diluted with water (200 mL) and extracted with DCM (2×100 mL). The combined organic phases were dried over magnesium sulfate and evaporated in vacuo. The crude material was purified by silica gel column chromatography eluting with a gradient of heptanes:EtOAc 100:0 to 70:30 to afford the title compound as a yellow oil (7.3 g, 74%).

LCMS (system 5): Rt=4.01 minutes MS m/z 436 [M+H]⁺

### Preparation 66

### 5-Iodo-7-{[2-(trimethylsilyl)ethoxy]methyl}-7H-pyrrolo[2,3-d]pyrimidine

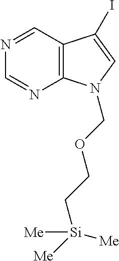 [SMILES: C[Si](C)(C)CCOCn1cc(I)c2cncnc21]

5-Iodo-7H-pyrrolo[2,3-d]pyrimidine (735 mg, 3.00 mmol) in DMF (5 mL) was added to sodium hydride (132 mg, 3.30 mmol, 60% in oil) in DMF (5 mL) at 0° C. The mixture was stirred at room temperature for 30 minutes then 2-(trimethylsilyl)ethoxymethyl chloride (0.58 mL, 3.30 mmol) was added at −20° C. The reaction mixture was stirred at −20° C. for 3 hours then water (30 mL) was added. The mixture was extracted with EtOAc (2×50 mL) and the combined organic phases were dried over magnesium sulphate and evaporated in vacuo. The crude solid was purified by silica gel column chromatography eluting with a gradient of Heptanes:EtOAc 100:0 to 50:50 to afford the title compound as a white solid in 61% yield, 691 mg.

¹H NMR (400 MHz, DMSO-d₆): δ ppm −0.11 (s, 9H), 0.81 (t, 2H), 3.51 (t, 2H), 5.61 (s, 2H), 8.01 (s, 1H), 8.77 (s, 1H), 8.89 (s, 1H).

### Preparation 67

### 7-(2-{[tert-Butyl(dimethyl)silyl]oxy}-1,1-dimethylethyl)-5-iodo-7H-pyrrolo[2,3-d]pyrimidine

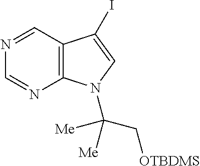 [SMILES: CC(C)(CO[Si](C)(C)C(C)(C)C)n1cc(I)c2cncnc21]

The title compound was prepared according to the method described for Preparation 85 using 2-(5-iodo-7H-pyrrolo[2,3-d]pyrimidin-7-yl)-2-methylpropan-1-ol (Preparation 69) to afford the title compound as a colourless oil in 74% yield, 8.20 g.

¹H NMR (400 MHz, CDCl₃): δ ppm 0.23 (s, 6H), 0.76 (s, 9H), 1.77 (s, 6H), 4.05 (s, 2H), 7.45 (s, 1H), 8.72 (s, 1H), 8.84 (s, 1H).

### Preparation 68

### 2-(2-Chloro-5-iodo-7H-pyrrolo[2,3-d]pyrimidin-7-yl)-2-methylpropan-1-ol

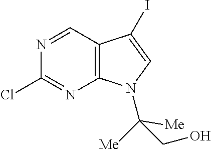 [SMILES: CC(C)(CO)n1cc(I)c2cnc(Cl)nc21]

Isobutyl chloroformate (6.6 mL, 50.02 mmol) was added to 2-(2-chloro-5-iodo-7H-pyrrolo[2,3-d]pyrimidin-7-yl)-2-methylpropanoic acid (Preparation 70, 16.6 g, 45.48 mmol) and triethylamine (12.64 mL, 90.9 mmol) in THF (300 mL) at 0° C. under nitrogen. The mixture was stirred at room temperature for 3 hours then filtered through a short plug of Celite™. The filtrate was cooled to 0° C. and sodium borohydride (8.6 g, 227.6 mmol) in water (300 mL) was added. The mixture was stirred for 10 minutes at 0° C., extracted with ethyl acetate (3×150 mL) then the organic extract was washed with brine (150 mL) and dried over sodium sulfate. The solution was evaporated in vacuo and the residue was triturated with hexane to afford the title compound as a white solid in 63% yield, 10 g.

¹H NMR (400 MHz, DMSO-d₆): δ ppm 1.64 (s, 6H), 3.85 (d, 2H), 4.99 (t, 1H), 7.82 (s, 1H), 8.63 (s, 1H).

### Preparation 69

### 2-(5-Iodo-7H-pyrrolo[2,3-d]pyrimidin-7-yl)-2-methylpropan-1-ol

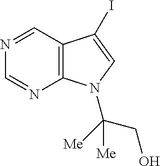 [SMILES: CC(C)(CO)n1cc(I)c2cncnc21]

Lithium borohydride (32.3 mL, 64.6 mmol, 2.0 M in THF) was added to methyl 2-(5-iodo-7H-pyrrolo[2,3-d]pyrimidin-7-yl)-2-methylpropanoate (Preparation 72, 8.92 g, 25.9 mmol) in ethanol (70 mL). The mixture was stirred at room temperature for 17 hours then water (70 mL) was added. The mixture was evaporated in vacuo then the residue was partitioned between DCM (250 mL) and water (50 mL). The aqueous phase was extracted with DCM:MeOH (90:10, 2×250 mL) and the combined organic phases were dried over magnesium sulphate and evaporated in vacuo to afford the title compound as an off-white solid in 100% yield, 8.20 g.

¹H NMR (400 MHz, DMSO-d₆): δ ppm 1.65 (s, 6H), 3.16 (d, 2H), 7.77 (s, 1H), 8.67 (s, 1H), 8.80 (s, 1H).

### Preparation 70

### 2-(2-Chloro-5-iodo-7H-pyrrolo[2,3-d]pyrimidin-7-yl)-2-methylpropanoic acid

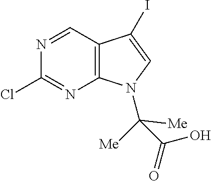 [SMILES: CC(C)(C(=O)O)n1cc(I)c2cnc(Cl)nc21]

Lithium hydroxide monohydrate (4.08 g, 97.5 mmol) was added to methyl 2-(2-chloro-5-iodo-7H-pyrrolo[2,3-d]pyrimidin-7-yl)-2-methylpropanoate (Preparation 71, 18.5 g, 48.7 mmol) in THF (185 mL) and water (45 mL) and the reaction was stirred at 60° C. for 3 hours. The reaction mixture volume was reduced to one third by evaporation in vacuo. The aqueous residue was acidified using aqueous HCl (2.0 M) and extracted with EtOAc (4×200 mL). The organic phase was evaporated in vacuo and the crude material was triturated with hexane (100 mL) to afford the title compound as a white solid in 90% yield, 16.0 g.

LCMS (system 5): Rt=2.24 minutes MS m/z 366 [M+H]⁺

### Preparation 71

### Methyl 2-(2-chloro-5-iodo-7H-pyrrolo[2,3-d]pyrimidin-7-yl)-2-methylpropanoate

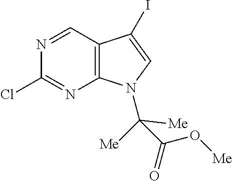 [SMILES: COC(=O)C(C)(C)n1cc(I)c2cnc(Cl)nc21]

Methyl 2-bromo-2-methylpropanoate (663 mL, 5.13 mmol) was added to 2-chloro-5-iodo-7H-pyrrolo[2,3-d]pyrimidine (Preparation 74, 358.1 g, 1.28 mol), potassium iodide (21.3 g, 128 mmol) and cesium carbonate (1670 g, 5.13 mol) in DMF (7162 mL). The mixture was heated at 60° C. for 19 hours. The reaction mixture was diluted with water (7000 mL) and stirred at room temperature for 42 hours. The mixture was filtered and the solid was washed with water (500 mL) to afford the title compound as a beige solid in 92% yield, 445.8 g.

¹H NMR (400 MHz, CDCl₃): δ ppm 1.89 (s, 6H), 3.65 (s, 3H), 7.39 (s, 1H), 8.56 (s, 1H).

### Preparation 72

### Methyl 2-(5-iodo-7H-pyrrolo[2,3-d]pyrimidin-7-yl)-2-methylpropanoate

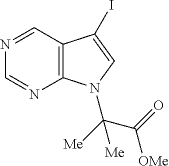 [SMILES: COC(=O)C(C)(C)n1cc(I)c2cncnc21]

Potassium t-butoxide (71.3 mL, 71.3 mmol, 1.0 M in THF) was added to (R,S) methyl 2-(5-iodo-7H-pyrrolo[2,3-d]pyrimidin-7-yl)propanoate (Preparation 73, 16.9 g, 50.9 mmol) and iodomethane (4.44 mL, 71.3 mmol) in THF (100 mL). The mixture was stirred at room temperature for 15 minutes then water (20 mL) and aqueous HCl (0.3 mL, 2M) were added. THF was removed by evaporation in vacuo then the aqueous residue was extracted with EtOAc (250 mL). The organic phase was dried over magnesium sulphate and evaporated in vacuo. The crude solid was purified by silica gel column chromatography eluting with 80:20 pentane:EtOAc to afford the title compound as a white solid in 51% yield, 8.92 g.

¹H NMR (400 MHz, CDCl₃): δ ppm 1.93 (s, 6H), 3.68 (s, 3H), 7.43 (s, 1H), 8.75 (s, 1H), 8.85 (s, 1H).

### Preparation 73

### (R,S) Methyl 2-(5-iodo-7H-pyrrolo[2,3-d]pyrimidin-7-yl)propanoate

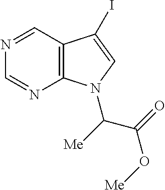 [SMILES: COC(=O)C(C)n1cc(I)c2cncnc21]

The title compound was prepared according to the method described for Preparation 79 using 5-iodo-7H-pyrrolo[2,3-d]pyrimidine and methyl-2-bromopropionate to afford the title compound as an off-white solid in 83% yield, 16.87 g.

¹H NMR (400 MHz, CDCl₃): δ ppm 1.82 (d, 3H), 3.76 (s, 3H), 5.72 (q, 1H), 7.48 (s, 1H), 8.76 (s, 1H), 8.89 (s, 1H).

### Preparation 74

### 2-Chloro-5-iodo-7H-pyrrolo[2,3-d]pyrimidine

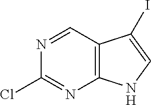 [SMILES: Clc1ncc2c(I)c[nH]c2n1]

N-Iodosuccinimide (742 g, 3.30 mol) was added to 2-chloro-7H-pyrrolo[2,3-d]pyrimidine (482.5 g, 3.14 mol) in acetonitrile (2500 mL) at 12° C. The mixture was stirred at room temperature for 1 hour then sodium metabisulphite (650 g in 4500 mL of water) was added. The mixture was stirred for 1 hour then filtered to afford the title compound as a orange solid in 82% yield, 716.2 g.

¹H NMR (400 MHz, DMSO-d₆): δ ppm 7.83 (s, 1H), 8.63 (s, 1H), 12.73 (s, 1H).

### Preparation 75

### 7-(trans-4-{[tert-butyl(dimethyl)silyl]oxy}cyclohexyl)-5-iodo-7H-pyrrolo[2,3-d]pyrimidine

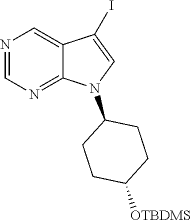 [SMILES: CC(C)(C)[Si](C)(C)O[C@H]1CC[C@H](n2cc(I)c3cncnc32)CC1]

A mixture of 7-(trans-4-{[tert-butyl(dimethyl)silyl]oxy}cyclohexyl)-7H-pyrrolo[2,3-d]pyrimidine (Preparation 98, 1455 mg, 4.39 mmol) and NIS (1180 mg, 5.27 mmol) in MeCN (22 mL) was stirred at room temperature for 16 hours followed by heating to 60° C. for 4 hours. The reaction was cooled and partitioned between water (100 mL) and EtOAc (100 mL). The aqueous layer was washed twice with EtOAc, the organic layers combined, dried over MgSO₄ and concentrated in vacuo. The residue was purified using silica gel column chromatography eluting with a gradient of heptanes:ethyl acetate 100:0 to 60:40 to afford the title compound as a gum (1180 mg, 59%).

¹H NMR (400 MHz, DMSO-d₆): δ ppm 0.08 (s, 6H), 0.88 (s, 9H), 1.41-1.58 (m, 2H), 1.83-2.10 (m, 6H), 3.69-3.82 (m, 1H), 4.59-4.74 (m, 1H), 8.00 (s, 1H), 8.71 (s, 1H), 8.83 (s, 1H).

### Preparation 76

### 7-Cyclopropyl-5-iodo-7H-pyrrolo[2,3-d]pyrimidine

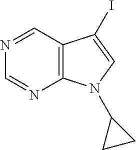 [SMILES: Ic1cn(C2CC2)c2ncncc12]

The title compound was prepared according to the method described for Preparation 80 using 7-cyclopropyl-7H-pyrrolo[2,3-d]pyrimidine (Preparation 103) to afford the title compound as a brown oil in 59% yield, 2.35 g.

¹H NMR (300 MHz, CDCl₃) δ: 1.1-2.2 (m, 4H), 3.5 (m, 1H), 7.2 (s, 1H), 8.7 (s, 1H), 9.0 (s, 1H).

### Preparation 77

### 7-(cis-4-{[tert-butyl(dimethyl)silyl]oxy}cyclohexyl)-5-iodo-7H-pyrrolo[2,3-d]pyrimidine

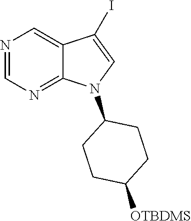 [SMILES: CC(C)(C)[Si](C)(C)O[C@H]1CC[C@@H](n2cc(I)c3cncnc32)CC1]

Prepared according to the method described for the trans isomer Preparation 75 using 7-(cis-4-{[tert-butyl(dimethyl)silyl]oxy}cyclohexyl)-7H-pyrrolo[2,3-d]pyrimidine (Preparation 104).

¹H NMR (400 MHz, DMSO-d₆): δ ppm 0.07 (s, 6H), 0.92 (s, 9H), 1.61-1.81 (m, 6H), 2.10-2.30 (m, 2H), 4.02-4.10 (m, 1H), 4.58-4.73 (m, 1H), 7.89 (s, 1H), 8.71 (s, 1H), 8.81 (s, 1H).

LCMS Rt=4.58 minutes MS m/z 458 [M+H]⁺

### Preparation 78

### 2-Chloro-5-iodo-7-isopropyl-7H-pyrrolo[2,3-d]pyrimidine

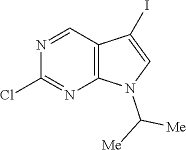 [SMILES: CC(C)n1cc(I)c2cnc(Cl)nc21]

2-Iodopropane (2.1 mL, 19.68 mmol) was added to a mixture of 2-chloro-5-iodo-7H-pyrrolo[2,3-d]pyrimidine (5.50 g, 19.8 mmol) and cesium carbonate (19.18 g, 59.04 mmol) in DMF (30 mL). The mixture was stirred at room temperature for 3 hours. The reaction mixture was diluted with EtOAc, washed with water, brine, dried over sodium sulfate and concentrated in vacuo. The residue was purified using silica gel column chromatography eluting with 10% EtOAc in hexane to afford the title compound as a white solid (5.5 g, 87%).

¹H NMR (400 MHz, CDCl₃): δ ppm 1.50 (d, 6H), 5.10 (m, 1H), 7.36 (s, 1H), 8.55 (s, 1H).

LCMS (System 10) Rₜ=3.6 minutes MS m/z 322 [M+H]⁺

### Preparation 79

### 5-Iodo-7-isopropyl-7H-pyrrolo[2,3-d]pyrimidine

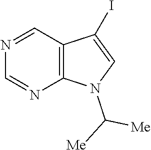 [SMILES: CC(C)n1cc(I)c2cncnc21]

2-Iodopropane (1.78 mL, 17.8 mmol) was added to a mixture of 5-iodo-7H-pyrrolo[2,3-d]pyrimidine (2.90 g, 12.0 mmol) and cesium carbonate (5.78 g, 17.8 mmol) in DMF (45 mL). The mixture was stirred at room temperature for 3 hours. The reaction mixture was poured into saturated aqueous ammonium chloride (500 mL) causing a solid to precipitate. The solid was collected by filtration, rinsed with water (200 mL) and dried in vacuo for 17 hours to afford the title compound as a brown solid in 77% yield, 2.61 g.

¹H NMR (400 MHz, CDCl₃): δ ppm 1.53 (d, 6H), 5.15 (m, 1H), 7.40 (s, 1H), 8.73 (s, 1H), 8.88 (s, 1H).

LCMS (system 2): Rt=1.02 minutes MS m/z 288 [M+H]⁺

### Preparation 80

### 7-tert-Butyl-5-iodo-7H-pyrrolo[2,3-d]pyrimidine

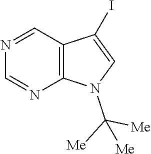 [SMILES: CC(C)(C)n1cc(I)c2cncnc21]

N-Iodosuccinimide (1.72 g, 7.63 mmol) was added to 7-tert-butyl-7H-pyrrolo[2,3-d]pyrimidine (Preparation 106, 1.27 g, 7.27 mmol) in acetonitrile (36.5 mL). The mixture was stirred at room temperature for 16 hours. The reaction mixture was evaporated in vacuo and the residue was dissolved in DCM (50 mL). The solution was washed with saturated aqueous sodium thiosulphate (50 mL), the organic phase was dried over magnesium sulfate and evaporated in vacuo. The crude material was purified by silica gel column chromatography eluting with a gradient of heptanes:EtOAc 100:0 to 40:60 to afford the title compound as a yellow solid in 71% yield, 1.55 g.

¹H NMR (400 MHz, DMSO-d₆): δ ppm 1.73 (s, 9H), 7.85 (s, 1H), 8.70 (s, 1H), 8.89 (s, 1H).

LCMS (system 1): Rt=3.12 minutes MS m/z 302 [M+H]⁺

### Preparation 81

### 5-Iodo-7-[2-(tetrahydro-2H-pyran-2-yloxy)ethyl]-7H-pyrrolo[2,3-d]pyrimidine

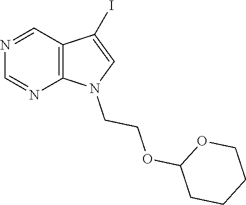 [SMILES: Ic1cn(CCOC2CCCCO2)c2ncncc12]

The title compound was prepared according to the method described for Preparation 79 using 5-iodo-7H-pyrrolo[2,3-d]pyrimidine and 2-(2-bromo-ethoxy)-tetrahydro-pyran to afford the title compound as a yellow oil in 74% yield, 22.6 g.

¹H NMR (400 MHz, DMSO-d₆): δ ppm 1.44-1.57 (m, 4H), 1.63-1.77 (m, 2H), 3.40 (m, 1H), 3.54 (m, 1H), 3.71 (m, 1H), 4.03 (m, 1H), 4.44 (m, 1H), 4.50-4.54 (m, 2H), 7.47 (s, 1H), 8.73 (s, 1H), 8.87 (s, 1H).

LCMS (System 9): Rt=3.16 minutes MS m/z 374 [M+H]⁺

### Preparation 82

### 7-(2-{[tert-Butyl(dimethyl)silyl]oxy}-1,1-dimethylethyl)-5-iodo-7H-pyrrolo[2,3-d]pyrimidine

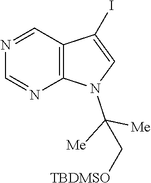 [SMILES: CC(C)(CO[Si](C)(C)C(C)(C)C)n1cc(I)c2cncnc21]

The title compound was prepared according to the method described for Preparation using 7-(2-{[tert-butyl(dimethyl)silyl]oxy}-1,1-dimethylethyl)-7H-pyrrolo[2,3-d]pyrimidine (Preparation 107) to afford the title compound as a brown oil in 59% yield, 270 mg

¹H NMR (400 MHz, CDCl₃): δ ppm 0.01 (s, 6H), 0.92 (s, 9H), 2.00 (s, 6H), 4.20 (s, 2H), 7.95 (s, 1H), 9.02 (s, 1H), 9.16 (s, 1H).

### Preparation 83

### 4-chloro-7-(3-methyloxetan-3-yl)-7H-pyrrolo[2,3-d]pyrimidine

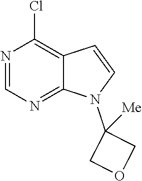 [SMILES: CC1(n2ccc3c(Cl)ncnc32)COC1]

n-BuLi (3.2 mL, 8.0 mmol, 2.5 M solution in hexane solution) was added to a solution of 2-(4-chloro-7H-pyrrolo[2,3-d]pyrimidin-7-yl)-2-methylpropane-1,3-diol (Preparation 84, 1930 mg, 8.0 mmol) in THF (30 mL) at −78° C. The reaction mixture was allowed to warm to −50° C. over 2 hours before TsCl (1530 mg, 8.0 mmol) in THF (10 mL) was added. The reaction was allowed to warm to 0° C. in 3 hours and additional n-BuLi (3.2 mL, 8.0 mmol, 2.5 M solution in hexane solution) was slowly added to the reaction mixture. The mixture was stirred for 1 hour at 0° C. and then stirred at 60° C. for 16 hours. After cooling to room temperature, the reaction was quenched by the addition of saturated aqueous NH₄Cl solution (20 mL) and water (40 mL), and the mixture was extracted with ethyl acetate thrice (3×50 mL). The organic layer was collected, dried over MgSO₄ and concentrated in vacuo. The crude residue was purified by silica gel column chromatography eluting with a gradient of 0-50% EtOAc in heptanes to afford the title compound that was used directly in the next reaction.

### Preparation 84

### 2-(4-chloro-7H-pyrrolo[2,3-d]pyrimidin-7-yl)-2-methylpropane-1,3-diol

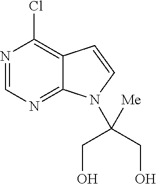 [SMILES: CC(CO)(CO)n1ccc2c(Cl)ncnc21]

The title compound was prepared according to Preparation 93 using 2-amino-2-methylpropane-1,3-diol. The solid was dissolved in MeOH (200 mL) and 1N aqueous HCl solution (100 mL) was added. The mixture was stirred for 30 minutes and concentrated in vacuo to remove MeOH and water to precipitate a solid. Water (100 mL) was added to the solid, followed by filtration and drying under vacuum.

¹HNMR (400 MHz, DMSO-d₆): δ ppm 1.66 (s, 3H), 3.86 (dd, 2H), 4.13 (dd, 2H), 4.92 (t, 2H), 6.58 (d, 1H), 7.73 (d, 1H), 8.59 (s, 1H).

### Preparation 85

### 7-(trans-4-{[tert-butyl(dimethyl)silyl]oxy}cyclohexyl)-4-chloro-7H-pyrrolo[2,3-d]pyrimidine

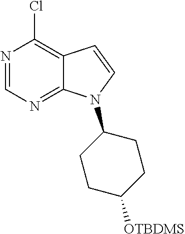 [SMILES: CC(C)(C)[Si](C)(C)O[C@H]1CC[C@H](n2ccc3c(Cl)ncnc32)CC1]

To a solution of trans-4-(4-chloro-7H-pyrrolo[2,3-d]pyrimidin-7-yl)cyclohexanol (Preparation 88, 1253 mg, 4.98 mmol) in DMF (16 mL) was added imidazole (847 mg, 12.4 mmol) and TBDMSCI (910 mg, 5.97 mmol) and the reaction was stirred at room temperature for 18 hours. The reaction was partitioned between water (100 mL) and EtOAc (100 mL), the aqueous layer was washed with EtOAc twice (2×100 mL), the organic layers combined, dried over MgSO₄ and concentrated in vacuo. The residue was purified using silica gel column chromatography eluting with a gradient of heptanes:ethyl acetate 100:0 to 60:40 to afford the title compound as a solid (1594 mg, 87%).

¹H NMR (400 MHz, DMSO-d₆): δ ppm 0.07 (s, 6H), 0.88 (s, 9H), 1.41-1.59 (m, 2H), 1.83-2.11 (m, 6H), 3.69-3.83 (m, 1H), 4.58-4.72 (m, 1H), 6.65 (d, 1H), 7.87 (d, 1H), 8.62 (s, 1H).

### Preparation 86

### 7-(cis-4-{[tert-butyl(dimethyl)silyl]oxy}cyclohexyl)-4-chloro-7H-pyrrolo[2,3-d]pyrimidine

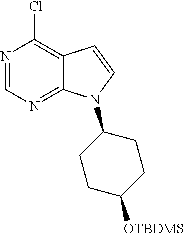 [SMILES: CC(C)(C)[Si](C)(C)O[C@H]1CC[C@@H](n2ccc3c(Cl)ncnc32)CC1]

Prepared according to the method described for the trans isomer Preparation 85 using cis-4-(4-chloro-7H-pyrrolo[2,3-d]pyrimidin-7-yl)cyclohexanol (Preparation 92).

¹H NMR (400 MHz, DMSO-d₆): δ ppm 0.08 (s, 6H), 0.92 (s, 9H), 1.62-1.84 (m, 6H), 2.11-2.29 (m, 2H), 4.04-4.12 (m, 1H), 4.59-4.75 (m, 1H), 6.65 (d, 1H), 7.75 (d, 1H), 8.61 (s, 1H).

### Preparation 87

### 7-(2-{[tert-butyl(dimethyl)silyl]oxy}-1-{[(tert-butylsilyl)oxy]methyl}-1-methylethyl)-4-chloro-7H-pyrrolo[2,3-d]pyrimidine

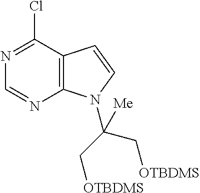 [SMILES: CC(CO[Si](C)(C)C(C)(C)C)(CO[Si](C)(C)C(C)(C)C)n1ccc2c(Cl)ncnc21]

The title compound was prepared according to Preparation 88 below using (4,6-dichloropyrimidin-5-yl)acetaldehyde and 2,2,3,3,6,9,9,10,10-Nonamethyl-4,8-dioxa-3,9-disilaundecan-6-amine (Preparation 110) as a colourless gum in 75% yield, 8.91 g.

¹H NMR (400 MHz, CDCl₃): δ ppm 0.09-0.11 (m, 12H), 0.78-0.79 (m, 18H), 1.74 (s, 3H), 4.06-4.09 (m, 2H), 4.29-4.31 (m, 2H), 6.52 (m, 1H), 7.44-7.45 (m, 1H), 8.56 (m, 1H).

### Preparation 88

### trans-4-(4-chloro-7H-pyrrolo[2,3-d]pyrimidin-7-yl)cyclohexanol

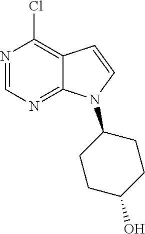 [SMILES: O[C@H]1CC[C@H](n2ccc3c(Cl)ncnc32)CC1]

To a solution of (4,6-dichloropyrimidin-5-yl)acetaldehyde (955 mg, 5 mmol) in EtOH (50 mL) was added trans-aminocyclohexanol (1150 mg, 10 mmol) and the reaction heated to 80° C. for 16 hours. The reaction was cooled and concentrated in vacuo. The reaction was partitioned between saturated aqueous ammonium chloride solution (50 mL) and EtOAc (50 mL), the aqueous layer was washed with EtOAc twice (2×100 mL), the organic layers combined, dried over MgSO₄ and concentrated in vacuo to afford the title compound (1260 mg, 99%).

¹H NMR (400 MHz, DMSO-d₆): δ ppm 1.32-1.50 (m, 2H), 1.82-2.05 (m, 6H), 3.49-3.62 (m, 1H), 4.57-4.69 (m, 1H), 4.71 (d, 1H), 6.66 (d, 1H), 7.87 (d, 1H), 8.62 (s, 1H).

### Preparation 89

### (2R)-2-(4-chloro-7H-pyrrolo[2,3-d]pyrimidin-7-yl)propan-1-ol

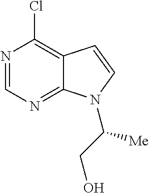

The title compound was prepared according to Preparation 88 using (4,6-dichloropyrimidin-5-yl)acetaldehyde and (2R)-2-aminopropan-1-ol.

LCMS Rt=0.89 minutes

### Preparation 90

### (2S)-2-(4-chloro-7H-pyrrolo[2,3-d]pyrimidin-7-yl)propan-1-ol

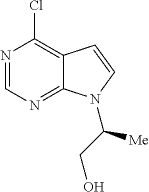 [SMILES: C[C@@H](CO)n1ccc2c(Cl)ncnc21]

The title compound was prepared according to Preparation 88 using (4,6-dichloropyrimidin-5-yl)acetaldehyde and (2S)-2-aminopropan-1-ol.

### Preparation 91

### 7-(2-{[tert-butyl(dimethyl)silyl]oxy}-1-methylethyl)-4-chloro-7H-pyrrolo[2,3-d]pyrimidine

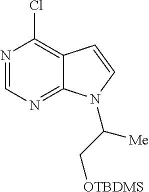 [SMILES: CC(CO[Si](C)(C)C(C)(C)C)n1ccc2c(Cl)ncnc21]

The title compound was prepared according to Preparation 88 using (4,6-dichloropyrimidin-5-yl)acetaldehyde and tert-butyl(dimethyl)silyl]oxy}-1-methylethylamine. The crude residue was used directly in the next step.

### Preparation 92

### cis-4-(4-chloro-7H-pyrrolo[2,3-d]pyrimidin-7-yl)cyclohexanol

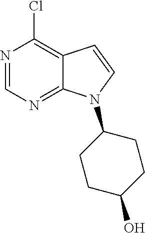 [SMILES: O[C@H]1CC[C@@H](n2ccc3c(Cl)ncnc32)CC1]

Prepared according to the method described for the trans isomer Preparation 88 using cis-aminocyclohexanol.

¹H NMR (400 MHz, DMSO-d₆): δ ppm 1.58-1.88 (m, 6H), 2.14-2.30 (m, 2H), 3.87-3.96 (m, 1H), 4.55 (d, 1H), 4.59-4.72 (m, 1H), 6.65 (d, 1H), 7.87 (d, 1H), 8.62 (s, 1H).

### Preparation 93

### 7-tert-Butyl-4-chloro-7H-pyrrolo[2,3-d]pyrimidine

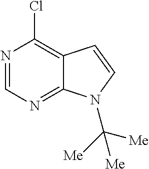 [SMILES: CC(C)(C)n1ccc2c(Cl)ncnc21]

tert-Butylamine (2.11 mL, 20.0 mmol) was added to (4,6-dichloropyrimidin-5-yl)acetaldehyde (1.91 g, 10.0 mmol) in ethanol (100 mL). The mixture was heated at 80° C. for 16 hours. The reaction mixture was evaporated in vacuo then the residue was diluted with water (100 mL) and extracted with ethyl acetate (3×100 mL). The combined organic extracts were evaporated in vacuo and the crude material was purified by silica gel column chromatography eluting with a gradient of Heptanes:EtOAc 100:0 to 60:40 to afford the title compound as a yellow liquid in 77% yield, 1.61 g.

¹H NMR (400 MHz, DMSO-d₆): δ ppm 1.75 (s, 9H), 6.60 (d, 1H), 7.79 (d, 1H), 8.63 (s, 1H).

### Preparation 94

### 4-Chloro-7-cyclopropyl-7H-pyrrolo[2,3-d]pyrimidine

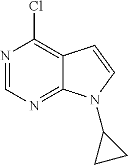 [SMILES: Clc1ncnc2c1ccn2C1CC1]

Cyclopropylamine (1.35 mL, 19.4 mmol) was added to 4,6-dichloro-5-(2,2-diethoxyethyl)pyrimidine (Preparation 142, 4.47 g, 17.70 mmol) and triethylamine (2.5 mL, 17.7 mmol) in ethanol (100 mL). The mixture was stirred at reflux for 10 hours. The reaction mixture was evaporated in vacuo. Water (40 mL) was added then extracted with ethyl acetate (50 mL) 3 times. The organic phase was dried over sodium sulphate and evaporated in vacuo.

The residue was dissolved in tetrahydrofuran (100 mL) and 2M hydrochloric acid (47 mL) was added and the solution stirred at reflux for 1 hour. The reaction mixture was evaporated in vacuo. The residue was dissolved in water (30 mL) and basified (pH-10) with saturated sodium hydroxide solution. The aqueous phase was extracted with ethyl acetate (50 mL). The organic phase was dried over sodium sulphate and evaporated in vacuo to afford the title compound as a colourless oil in 81% yield, 2.8 g.

¹H NMR (300 MHz, DMSO-d₆): δ ppm 0.9-1.1 (d, 4H), 3.65 (m, 1H), 6.6 (m, 1H), 7.6 (m, 1H) 8.7 (s, 1H).

### Preparation 95

### 4-chloro-7-[(1R)-1-methyl-2-(tetrahydro-2H-pyran-2-yloxy)ethyl]-7H-pyrrolo[2,3-d]pyrimidine

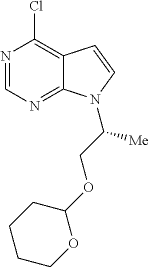 [SMILES: C[C@H](COC1CCCCO1)n1ccc2c(Cl)ncnc21]

To a solution of (2R)-2-(4-chloro-7H-pyrrolo[2,3-d]pyrimidin-7-yl)propan-1-ol (Preparation 89, 11 g, 51.97 mmol) in DCM (44 mL) was added dihydropyran (23 g, 270 mmol) and PPTS (3.92 g, 15.6 mmol) and the reaction was stirred at room temperature for 18 hours. The reaction was washed with water (200 mL) and the aqueous layer washed with DCM (2×150 mL). The combined organic layers were dried over MgSO₄ and concentrated in vacuo. The residue was purified using silica gel column chromatography eluting with 0-30% EtOAc in heptanes to afford the title compound.

LCMS (2 minutes) Rt=1.73 minutes MS m/z 296 [M+H]+

### Preparation 96

### 4-chloro-7-[(1S)-1-methyl-2-(tetrahydro-2H-pyran-2-yloxy)ethyl]-7H-pyrrolo[2,3-d]pyrimidine

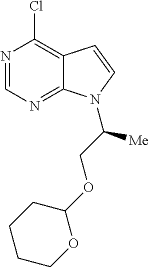 [SMILES: C[C@@H](COC1CCCCO1)n1ccc2c(Cl)ncnc21]

The title compound was prepared according to Preparation 95 using the (S) enantiomer (Preparation 90).

### Preparation 97

### 7-(2-{[tert-Butyl(dimethyl)silyl]oxy}-1,1-dimethylethyl)-4-chloro-7H-pyrrolo[2,3-d]pyrimidine

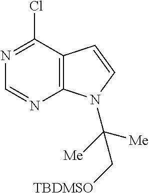 [SMILES: CC(C)(CO[Si](C)(C)C(C)(C)C)n1ccc2c(Cl)ncnc21]

The title compound was prepared according to the method described for Preparation 93 using (4,6-dichloropyrimidin-5-yl)acetaldehyde and 1-{[tert-butyl(dimethyl)silyl]oxy}-2-methylpropan-2-amine to afford the title compound as a yellow oil in 38% yield, 377 mg.

¹H NMR (400 MHz, CDCl₃) δ: 0.00 (s, 6H), 0.94 (s, 9H), 1.97 (s, 6H), 4.28 (s, 2H), 6.78 (d, 1H), 7.66 (d, 1H), 8.83 (s, 1H).

### Preparation 98

### 7-(trans-4-{[tert-butyl(dimethyl)silyl]oxy}cyclohexyl)-7H-pyrrolo[2,3-d]pyrimidine

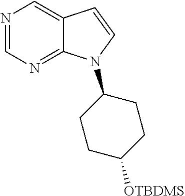 [SMILES: CC(C)(C)[Si](C)(C)O[C@H]1CC[C@H](n2ccc3cncnc32)CC1]

To a solution of 7-(trans-4-{[tert-butyl(dimethyl)silyl]oxy}cyclohexyl)-4-chloro-7H-pyrrolo[2,3-d]pyrimidine (Preparation 85, 1594 mg, 4.355 mmol) in EtOH (22 mL) was added 880 NH₃ (2.2 mL) and palladium on carbon (150 mg, 1.5 mmol) and the reaction was hydrogenated at 20 psi at room temperature for 18 hours. The reaction was filtered through Arbocel and concentrated in vacuo. The residue was partitioned between water (100 mL) and EtOAc (100 mL), the aqueous layer was washed with EtOAc twice (2×100 mL), the organic layers combined, dried over MgSO₄ and concentrated in vacuo to afford a yellow solid that was used directly in the next reaction.

¹H NMR (400 MHz, DMSO-d₆): δ ppm 0.08 (s, 6H), 0.88 (s, 9H), 1.41-1.58 (m, 2H), 1.81-2.11 (m, 6H), 3.70-3.83 (m, 1H), 4.58-4.72 (m, 1H), 6.62 (d, 1H), 7.73 (d, 1H), 8.77 (s, 1H), 8.97 (s, 1H).

### Preparation 99

### 7-[(1R)-1-methyl-2-(tetrahydro-2H-pyran-2-yloxy)ethyl]-7H-pyrrolo[2,3-d]pyrimidine

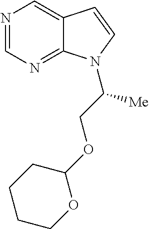 [SMILES: C[C@H](COC1CCCCO1)n1ccc2cncnc21]

The title compound was prepared according to Preparation 98 using 4-chloro-7-[(1R)-1-methyl-2-(tetrahydro-2H-pyran-2-yloxy)ethyl]-7H-pyrrolo[2,3-d]pyrimidine (Preparation 95).

LCMS Rt=0.73 minutes MS m/z 262 [M+H]⁺

### Preparation 100

### 7-[(1S)-1-methyl-2-(tetrahydro-2H-pyran-2-yloxy)ethyl]-7H-pyrrolo[2,3-d]pyrimidine

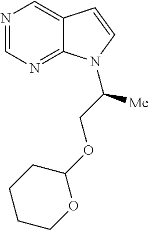 [SMILES: C[C@@H](COC1CCCCO1)n1ccc2cncnc21]

The title compound was prepared according to Preparation 98 using the (S) enantiomer (Preparation 96).

### Preparation 101

### 7-(2-{[tert-butyl(dimethyl)silyl]oxy}ethyl)-7H-pyrrolo[2,3-d]pyrimidine

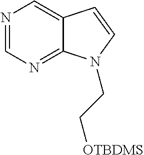 [SMILES: CC(C)(C)[Si](C)(C)OCCn1ccc2cncnc21]

The title compound was prepared according to Preparation 98 using tertbutyl(dimethyl)silyloxy-1-ethylamine. The crude residue was used directly in the next reaction.

### Preparation 102

### 7-(3-methyloxetan-3-yl)-7H-pyrrolo[2,3-d]pyrimidine

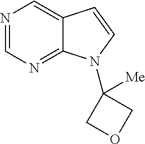 [SMILES: CC1(n2ccc3cncnc32)COC1]

The title compound was prepared according to Preparation 98 using 4-chloro-7-(3-methyloxetan-3-yl)-7H-pyrrolo[2,3-d]pyrimidine (Preparation 83). Purified using silica gel column chromatography eluting with a gradient of 40-100% EtOAc in heptanes.

¹HNMR (400 MHz, DMSO-d₆): δ ppm 1.81 (s, 3H), 4.72-4.77 (m, 2H), 5.16-5.21 (m, 2H), 6.69 (d, 1H), 7.66 (d, 1H), 8.76 (s, 1H), 9.02 (s, 1H).

### Preparation 103

### 7-Cyclopropyl-7H-pyrrolo[2,3-d]pyrimidine

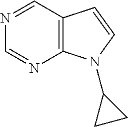 [SMILES: c1ncc2ccn(C3CC3)c2n1]

The title compound was prepared according to the method described for Preparation 106 using 4-chloro-7-cyclopropyl-7H-pyrrolo[2,3-d]pyrimidine (Preparation 94) to afford the title compound as a yellow foam in 91% yield, 2.1 g.

¹H NMR (300 MHz, CDCl₃) δ: 0.9-1.1 (m, 4H), 3.6 (m, 1H), 6.6 (m, 1H), 7.6 (m, 1H), 8.7 (s, 1H), 9.0 (s, 1H).

### Preparation 104

### 7-(cis-4-{[tert-butyl(dimethyl)silyl]oxy}cyclohexyl)-7H-pyrrolo[2,3-d]pyrimidine

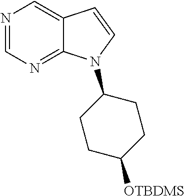 [SMILES: CC(C)(C)[Si](C)(C)O[C@H]1CC[C@@H](n2ccc3cncnc32)CC1]

Prepared according to the method described for the trans isomer Preparation 98 using 7-(cis-4-{[tert-butyl(dimethyl)silyl]oxy}cyclohexyl)-4-chloro-7H-pyrrolo[2,3-d]pyrimidine (Preparation 86). The residue was used directly in the next reaction.

### Preparation 105

### 7-(2-{[tert-butyl(dimethyl)silyl]oxy}-1-{[(tert-butylsilyl)oxy]methyl}-1-methylethyl)-7H-pyrrolo[2,3-d]pyrimidine

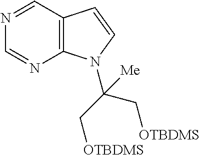 [SMILES: CC(CO[Si](C)(C)C(C)(C)C)(CO[Si](C)(C)C(C)(C)C)n1ccc2cncnc21]

Prepared according to Preparation 98 using 7-(2-{[tert-butyl(dimethyl)silyl]oxy}-1-{[(tert-butylsilyl)oxy]methyl}-1-methylethyl)-4-chloro-7H-pyrrolo[2,3-d]pyrimidine (Preparation 87).

¹HNMR (400 MHz, CDCl₃): δ ppm −0.13 (s, 6H), −0.10 (s, 6H), 0.78 (s, 18H), 1.75 (s, 3H), 4.11 (d, 2H), 4.33 (d, 2H), 6.45 (d, 1H), 7.41 (d, 1H, d), 8.78 (s, 1H), 8.90 (s, 1H).

### Preparation 106

### 7-tert-Butyl-7H-pyrrolo[2,3-d]pyrimidine

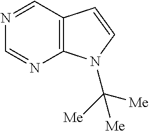 [SMILES: CC(C)(C)n1ccc2cncnc21]

Palladium (10% on carbon, 160 mg) was added to 7-tert-butyl-4-chloro-7H-pyrrolo[2,3-d]pyrimidine (Preparation 93, 1.61 g, 7.69 mmol) in ethanol (38 mL) and concentrated ammonia solution (3.8 mL) and hydrogenated (20 psi, 20° C.) for 18 hours. The reaction mixture was filtered through Arbocel™ and the filtrate was evaporated in vacuo. The residue was diluted with water (100 mL) and extracted with ethyl acetate (3×100 mL). The combined organic extracts were evaporated in vacuo to afford the title compound as a yellow liquid in 94% yield, 1.27 g.

¹H NMR (400 MHz, DMSO-d₆): δ ppm 1.75 (s, 9H), 6.57 (d, 1H), 7.66 (d, 1H), 8.78 (s, 1H), 8.98 (s, 1H).

### Preparation 107

### 7-(2-{[tert-Butyl(dimethyl)silyl]oxy}-1,1-dimethylethyl)-7H-pyrrolo[2,3-d]pyrimidine

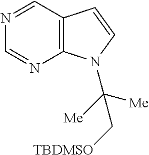 [SMILES: CC(C)(CO[Si](C)(C)C(C)(C)C)n1ccc2cncnc21]

The title compound was prepared according to the method described for Preparation 106 using 7-(2-{[tert-butyl(dimethyl)silyl]oxy}-1,1-dimethylethyl)-4-chloro-7H-pyrrolo[2,3-d]pyrimidine (Preparation 97) to afford the title compound as a white solid in 97% yield, 327 mg.

¹H NMR (400 MHz, CDCl₃): δ ppm 0.01 (s, 6H), 0.94 (s, 9H), 2.02 (s, 6H), 4.30 (s, 2H), 6.92 (d, 1H), 7.84 (d, 1H), 9.13 (s, 1H), 9.21 (s, 1H).

### Preparation 108

### 2-(3,5-Dimethyl-1H-pyrazol-1-yl)-N-[4-(7H-pyrrolo[2,3-d]pyrimidin-5-ylcarbonyl)pyridin-2-yl]acetamide

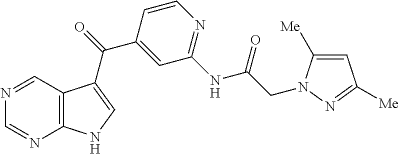 [SMILES: Cc1cc(C)n(CC(=O)Nc2cc(C(=O)c3c[nH]c4ncncc34)ccn2)n1]

Trifluoroacetic acid (10 mL) was added to 2-(3,5-dimethyl-1H-pyrazol-1-yl)-N-{4-[(7-{[2-(trimethylsilyl)ethoxy]methyl}-7H-pyrrolo[2,3-d]pyrimidin-5-yl)carbonyl]pyridin-2-yl}acetamide (Preparation 5, 485 mg, 0.96 mmol) and the mixture was stirred at room temperature for 1 hour. The reaction mixture was evaporated in vacuo then the residue was diluted with water (20 mL). Saturated aqueous sodium bicarbonate (20 mL) was added and a solid precipitated. The solid was collected by filtration and dried to afford the title compound as a white solid in 99% yield, 356 mg.

¹H NMR (400 MHz, DMSO-D₆): δ ppm 2.05 (s, 3H), 2.16 (s, 3H), 4.95 (s, 2H), 5.61 (s, 1H), 7.47 (m, 1H), 8.28 (s, 1H), 8.35 (s, 1H), 8.55 (m, 1H), 8.93 (s, 1H), 9.43 (s, 1H), 11.04 (s, 1H).

### Preparation 109

### 2-(4-Chlorophenyl)-N-[4-(7H-pyrrolo[2,3-d]pyrimidin-5-ylcarbonyl)pyridin-2-yl]acetamide

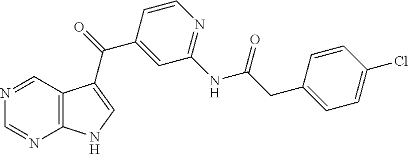 [SMILES: O=C(Cc1ccc(Cl)cc1)Nc1cc(C(=O)c2c[nH]c3ncncc23)ccn1]

The title compound was prepared according to the method described for Preparation 108 using 2-(4-chlorophenyl)-N-{4-[(7-{[2-(trimethylsilyl)ethoxy]methyl}-7H-pyrrolo[2,3-d]pyrimidin-5-yl)carbonyl]pyridin-2-yl}acetamide (Preparation 6) to afford the title compound as an off-white solid in 100% yield, 52 mg.

LCMS (system 2): Rt=1.08 minutes MS m/z 391, 393 [M+H]⁺

### Preparation 110

### 2,2,3,3,6,9,9,10,10-Nonamethyl-4,8-dioxa-3,9-disilaundecan-6-amine

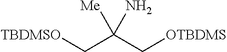 [SMILES: CC(N)(CO[Si](C)(C)C(C)(C)C)CO[Si](C)(C)C(C)(C)C]

The title compound was prepared according to the method described for Preparation 85 using 2-amino-2-methyl-1,3-propanediol to afford the title compound as a colourless oil in 100% yield, 23 g.

¹H NMR (400 MHz, CDCl₃): δ ppm 0.05 (s, 12H), 0.87-0.99 (m, 21H), 3.36-3.42 (m, 4H).

### Preparation 111

### 4-[(diphenylmethylene)amino]-N-methoxy-N-methylpyridine-2-carboxamide

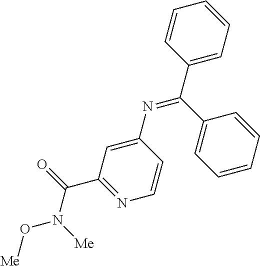 [SMILES: CON(C)C(=O)c1cc(N=C(c2ccccc2)c2ccccc2)ccn1]

A mixture of 4-chloro-pyridine-2-carboxylic acid methoxy-methylamide (Preparation 112, 10 g, 50 mmol), benzophenone imine (10 mL, 60 mmol), Pd₂(dba)₃ (1.37 g, 1.5 mmol), di-tert-butyl(2′,4′,6′-triisopropylbiphenyl-2-yl)phosphine (1.7 g, 4.0 mmol) and K₃PO₄ (26.5 g, 125 mmol) in DME (100 mL) was stirred at 50° C. for 16 hours. The reaction was cooled to room temperature, diluted with DCM (100 mL) and the mixture filtered through a pad of arbocel. The filter cake was rinsed with DCM (100 mL) and the filtrate was concentrated in vacuo. The crude residue was purified using silica gel column chromatography eluting with heptane:ethyl acetate 50:50 to 0:100 to afford the title compound as a gum (17.3 g, 84%).

¹HNMR (400 MHz, DMSO-d₆): δ ppm 3.16 (br s, 3H), 3.47 (br s, 3H), 6.81-6.91 (m, 2H), 7.12-7.76 (m, 10H), 8.29 (dd, 1H).

### Preparation 112

### 4-Chloro-pyridine-2-carboxylic acid methoxy-methyl-amide

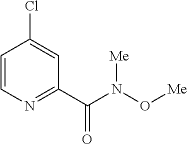 [SMILES: CON(C)C(=O)c1cc(Cl)ccn1]

To a solution of N,O-dimethyl hydroxyl amine hydrochloride (4.6 g, 47.61 mmol) in DMF (50 mL) was added DIPEA (16.9 mL, 95.23 mmol) at 0° C. and the mixture stirred for 10 minutes. 4-chloropyridine-2-carboxylic acid (5.0 g, 31.74 mmol) was then added followed by HATU (17.8 g, 47.61 mmol) and the reaction stirred at room temperature for 16 hours. The reaction was concentrated in vacuo and purified using silica gel column chromatography eluting with 15-20% EtOAc in hexane to afford the title compound (4.8 g, 96%).

¹H NMR (400 MHz, CDCl₃): δ ppm 3.37 (s, 3H), 3.72 (s, 3H), 7.35 (d, 1H), 7.63 (br s, 1H), 7.48 (d, 1H).

LCMS Rt=2.30 minutes MS m/z 201 [M+H]⁺

### Preparation 113

### 4-Chloro-pyridine-2-carboxylic acid methoxy-methyl-amide

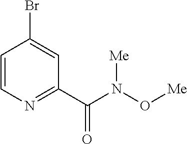 [SMILES: CON(C)C(=O)c1cc(Br)ccn1]

To a solution of 4-bromopyridine-2-carboxylic acid (5.0 g, 24.75 mmol) and HATU (18.81 g, 49.5 mmol) in DMF (30 mL) was added N,O-dimethyl hydroxyl amine hydrochloride (2.90 g, 29.70 mmol) and DIPEA (21 mL, 123.75 mmol). The reaction was stirred at room temperature for 16 hours. The reaction was diluted with water and extracted into EtOAc. The organic layer was collected, washed with water, brine, dried over sodium sulphate and concentrated in vacuo. The residue was purified using silica gel column chromatography eluting with 15-20% EtOAc in hexane to afford the title compound (5 g).

¹H NMR (400 MHz, DMSO-d₆): δ ppm 3.31 (s, 3H), 3.64 (s, 3H), 7.78 (d, 1H), 7.86 (s, 1H), 8.48 (d, 1H).

### Preparation 114

### 2-Chloro-N,6-dimethoxy-N-methyl-4-pyridinecarboxamide

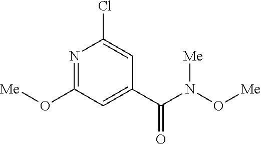 [SMILES: COc1cc(C(=O)N(C)OC)cc(Cl)n1]

2-Chloro-6-methoxyisonicotinic acid (1.00 g, 5.33 mmol) and N-Methoxymethylamine hydrochloride (572 mg, 5.86 mmol) were dissolved in DCM (20 mL) and triethylamine (2.23 mL, 16.0 mmol), HBTU (2.22 g, 5.86 mmol) was added and the resulting solution was stirred at room temperature under nitrogen for 18 hours. The reaction mixture was washed with saturated NaHCO₃ aq (20 mL), the aqueous layer was extracted with DCM (3×15 mL) and the combined organic layers were washed with brine (20 mL), dried over MgSO₄ and evaporated to obtain a colourless oil. The residue was purified by silica gel column chromatography eluting with 80:20-60:40 heptane:ethyl acetate to afford the title compound as a colourless oil (736 mg, 60%).

¹H NMR (400 MHz, CDCl₃): δ ppm 3.35 (s, 3H), 3.60 (s, 3H), 3.95 (s, 3H), 6.85 (d, 1H), 7.10 (d, 1H).

### Preparation 115

### 2-[(Diphenylmethylene)amino]-N-methoxy-N-methylisonicotinamide

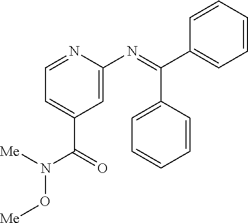 [SMILES: CON(C)C(=O)c1ccnc(N=C(c2ccccc2)c2ccccc2)c1]

Benzophenone imine (2.17 g, 12.0 mmol) was added to 2-bromo-N-methoxy-N-methylisonicotinamide (2.45 g, 10.0 mmol), tris(dibenzylideneacetone)dipalladium (458 mg, 0.50 mmol), 2-di-tert-butylphosphino-2′,4′,6′-triisopropylbiphenyl (552 mg, 1.30 mmol) and sodium t-butoxide (2.40 g, 25.0 mmol) in toluene (40 mL). The mixture was stirred at room temperature for 2 hours. The reaction mixture was diluted with DCM and filtered through Arbocel™. The filtrate was washed with water (100 mL) then the organic phase was dried over sodium sulphate and evaporated in vacuo. The crude material was purified by silica gel column chromatography eluting with a gradient of heptanes:EtOAc 100:0 to 30:70 to afford the title compound as an orange gum in 71% yield, 2.44 g.

¹H NMR (400 MHz, DMSO-d₆): δ ppm 3.14 (br s, 3H), 3.30 (br s, 3H), 6.76 (m, 1H), 7.02 (dd, 1H), 7.11-7.19 (m, 2H), 7.27-7.36 (m, 3H), 7.46-7.54 (m, 2H), 7.59 (m, 1H), 7.66-7.73 (m, 2H), 8.32 (dd, 1H).

### Preparation 116

### 4-Cyano-3-fluoro-N-methoxy-N-methyl-benzamide

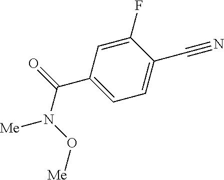 [SMILES: CON(C)C(=O)c1ccc(C#N)c(F)c1]

To a solution of N,O-dimethyl hydroxyl amine hydrochloride (443 mg, 4.54 mmol) in DMF (5 mL), DIPEA (1.61 mL, 9.09 mmol) was added under cooling condition (ice bath) and stirred at that temperature for 10 minutes. HATU (1.72 gm, 4.54 mmol) and 4-cyano-3-fluoro-benzoic acid (500 mg, 3.03 mmol) were added and resulting mixture was allowed to stir for 16 hours at room temperature. The reaction was diluted with EtOAc and the organic layer was washed with saturated NaHCO₃ solution, water, brine, dried over sodium sulphate and evaporated in vacuo. The crude material was purified by silica gel column chromatography eluting with a gradient of 10-25% EtOAc in Hexane to afford the title compound as off white solid in 71% yield, 450 mg.

¹H NMR (400 MHz, CDCl₃): δ ppm 3.36 (s, 3H), 3.53 (s, 3H), 7.52-7.57 (m, 2H), 7.67 (t, 1H).

LCMS Rt=3.02 minutes MS m/z 209 [M+H]⁺

### Preparation 117

### [4-(Trifluoromethyl)-1H-1,2,3-triazol-1-yl]acetic acid

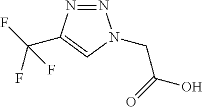 [SMILES: O=C(O)Cn1cc(C(F)(F)F)nn1]

Trifluoromethyl acetylene (22.0 g, 0.234 mol) in THF (210 mL) was added to sodium ascorbate (2.77 g, 14.0 mmol), ethyl azidoacetate (27.1 g, 0.210 mol) and copper sulfate (4.76 mL, 0.3 M in water) in water (105 mL). The mixture was stirred at room temperature for 240 hours then evaporated in vacuo. The residue was extracted with EtOAc (500 mL) and the organic phase was dried over magnesium sulfate then evaporated in vacuo.

Sodium hydroxide (7.32 g, 0.183 mol) in water (30 mL) was added to the residue (32.7 g, 0.146 mol) in methanol (50 mL) and the mixture was stirred at room temperature for 17 hours. The methanol was evaporated in vacuo and the residue was diluted with water (10 mL). Potassium hydrogen sulfate (26.6 g, 0.195 mol) in water (70 mL) was added. The solution was evaporated in vacuo and the crude solid was purified by crystallization using water to afford the title compound as a white solid in 75% yield, 25.8 g.

¹H NMR (400 MHz, DMSO-d₆): δ ppm 5.40 (s, 2H), 8.85 (s, 1H), 13.50 (br s, 1H).

### Preparation 118

### (5-isopropyl-1H-pyrazol-1-yl)acetic acid

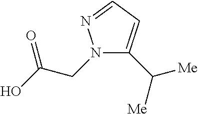 [SMILES: CC(C)c1ccnn1CC(=O)O]

Prepared according to the method described for Preparation 126 using (5-isopropyl-1H-pyrazol-1-yl)acetic acid ethyl ester (Preparation 119) in IMS.

MS m/z 169 [M+H]⁺

### Preparation 119

### (5-isopropyl-1H-pyrazol-1-yl)acetic acid ethyl ester

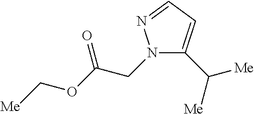 [SMILES: CCOC(=O)Cn1nccc1C(C)C]

Prepared according to the method described for Preparation 121 using 5-isopropyl-1H-pyrazole and ethyl bromoacetate. The residue was purified using silica gel column chromatography eluting with 4:1 hexane:EtOAc. The title compound was isolated as the lower running minor product in 6% yield, and taken directly on to the next step.

### Preparation 120

### Ethyl (4-cyclopropyl-1H-1,2,3-triazol-1-yl)acetate

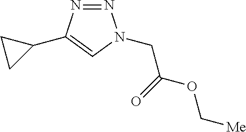 [SMILES: CCOC(=O)Cn1cc(C2CC2)nn1]

Cyclopropylacetylene (15 g, 0.116 mol), ethyl azidoacetate (11.5 g, 0.174 mol), triethylamine (0.32 mL, 2.33 mmol) and copper iodide (442 mg, 2.33 mmol) in acetonitrile (100 mL) were stirred at 25° C. for 18 hours. The mixture was evaporated in vacuo and the residue was partitioned between water (100 mL) and ethyl acetate (100 mL). The organic phase was dried over sodium sulfate, evaporated in vacuo and purified by silica gel column chromatography EtOAc:Hexane 40:60 to afford the title compound as a colorless liquid in 95% yield, 21.6 g.

¹H NMR (400 MHz, DMSO-d₆): δ ppm 0.68 (m, 2H), 0.90 (m, 2H), 1.21 (t, 3H), 1.95 (m, 1H), 4.17 (q, 2H), 5.29 (s, 2H), 7.81 (s, 1H).

### Preparation 121

### (4-Cyclopropyl-1H-1,2,3-triazol-1-yl)acetic acid

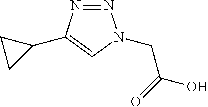 [SMILES: O=C(O)Cn1cc(C2CC2)nn1]

The title compound was prepared according to the method described for Preparation 126 using ethyl (4-cyclopropyl-1H-1,2,3-triazol-1-yl)acetate (Preparation 120) to afford the title compound as a yellow solid in 63% yield, 13.0 g.

LCMS (system 4): Rt=1.86 minutes MS m/z 186 [M+NH₄]⁺

### Preparation 122

### 1-oxa-spiro[2.2]pentane

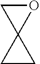

To a solution of methylenecyclopropane (1.32 g, 24.4 mmol) in DCM (20 mL) at 0° C. was added mcpba (5.33 g, 23.2 mmol) and the reaction stirred for 2 hours. The reaction was filtered and the filtrate collected, washed with water, saturated aqueous NaHCO₃ solution, dried over MgSO₄ and cooled to 5° C. for 18 hours. The resulting precipitate was filtered, the filtrate collected and concentrated in vacuo to afford the title compound.

¹H NMR (400 MHz, CDCl₃): δ ppm 1.00 (t, 2H), 1.15 (t, 2H), 3.29 (s, 2H).

### Preparation 123

### 2-{[tert-butyl(dimethyl)silyl]oxy}-1-({[tert-butyl(dimethyl)silyl]oxy}methyl)ethyl trifluoromethanesulfonate

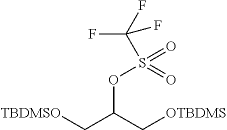 [SMILES: CC(C)(C)[Si](C)(C)OCC(CO[Si](C)(C)C(C)(C)C)OS(=O)(=O)C(F)(F)F]

To a stirred solution of 2,2,3,3,9,9,10,10-octamethyl-4,8-dioxa-3,9-disilaundecan-6-ol (Preparation 124, 6410 mg, 20 mmol) and pyridine (2.42 mL, 30 mmol) in DCM (40 mL) at −50° C. was added trifluoromethanesulfonic anhydride (5.05 mL, 30 mmol) and the reaction stirred at −30° C. for 2 hours. Aqueous 1N HCl (40 mL) was added to the reaction and the mixture was extracted with DCM (40 mL×3). The combined organic layers were concentrated under reduced pressure to obtain a colourless oil which was used in the next step without further purification.

### Preparation 124

### 2,2,3,3,9,9,10,10-octamethyl-4,8-dioxa-3,9-disilaundecan-6-ol

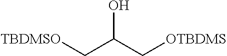 [SMILES: CC(C)(C)[Si](C)(C)OCC(O)CO[Si](C)(C)C(C)(C)C]

To a stirred solution of glycerol (4.01 mL, 55 mmol) and imidazole (18.7 g, 275 mmol) in DMF (150 mL) at 0° C. was added tert-butyldimethylsilyl chloride (17.2 g, 113 mmol) in DMF (33 mL). The reaction mixture was allowed to warm to room temperature and stirred for 16 hours. Water (500 mL) was added to the reaction mixture and the resulting mixture extracted with heptane (500 mL×3). The combined organic layers were washed with water (300 mL), dried over MgSO₄ and concentrated under reduced pressure. The crude material was purified by silica gel column chromatography eluting with a gradient of heptane:EtOAc 100:0 to 80:20 to afford the title compound as a colourless oil in 68% yield, 11.9 g.

1H NMR (400 MHz, DMSO-d₆) δ: 0.03 (s, 12H), 0.86 (s, 18H), 3.40-3.59 (m, 5H), 4.58 (d, 1H).

### Preparation 125

### {[3-(Bromomethyl)oxetan-3-yl]methoxy}(tert-butyl)dimethylsilane

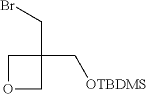 [SMILES: CC(C)(C)[Si](C)(C)OCC1(CBr)COC1]

The title compound was prepared according to the method described for Preparation 124 using [3-(bromomethyl)oxetan-3-yl]methanol to afford the title compound as a colourless oil in 100% yield, 2.45 g.

¹H NMR (400 MHz, CDCl₃): δ ppm 0.09 (s, 6H), 0.90 (s, 9H), 3.74 (s, 2H), 3.90 (s, 2H), 4.41 (br s, 4H).

### Preparation 126

### [3-(trifluoromethyl)-1H-pyrazol-1-yl]acetic acid

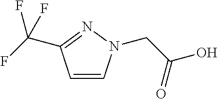 [SMILES: O=C(O)Cn1ccc(C(F)(F)F)n1]

Lithium hydroxide monohydrate (127 mg, 3.03 mmol) in water (0.5 mL) was added to (3-(trifluoromethyl)-1H-pyrazol-1-yl)acetic acid ethyl ester (250 mg, 1.125 mmol) in THF (5 mL). The mixture was stirred at room temperature for 5 hours then the reaction mixture volume was reduced to one third by evaporation in vacuo. The aqueous residue was acidified using aqueous HCl (2M) to pH5. The resulting off white solid was filtered, collected and dried, washed with ether to afford the title compound as a white solid (42 mg, 19%).

¹HNMR (400 MHz, DMSO-d₆): δ ppm 5.07 (s, 2H), 6.73 (s, 1H), 7.95 (s, 1H).

LCMS Rt=2.93 minutes MS m/z 195 [M+H]⁺

### Preparation 127

### Ethyl (3-cyclopropyl-1H-pyrazol-1-yl)acetate

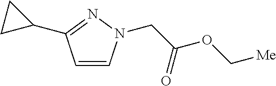 [SMILES: CCOC(=O)Cn1ccc(C2CC2)n1]

Potassium carbonate (7.67 g, 55.56 mmol) was added to 3-cyclopropyl-1H-pyrazole (2.0 g, 18.52 mmol) in dry DMF (20 mL) at 25° C. and the mixture was stirred for 20 minutes. Ethyl bromoacetate (2.06 mL, 18.52 mmol) was added then the mixture was stirred for 2 days at room temperature. The reaction mixture was neutralized with aqueous 1M HCl, extracted with ether (40 mL) and the organic extract was washed with brine (30 mL), dried over sodium sulfate then evaporated in vacuo. The residue was purified by silica gel column chromatography eluting with hexane:EtOAc 88:12 to afford the title compound as a yellow oil (42%, 1.50 g).

¹H NMR (400 MHz, DMSO-d₆): δ ppm 0.59 (d, 2H), 0.83 (d, 2H), 1.19 (t, 3H), 1.83 (m, 1H), 4.13 (q, 2H), 4.91 (s, 2H), 5.94 (d, 1H), 7.54 (d, 1H).

### Preparation 128

### (3-Cyclopropyl-1H-pyrazol-1-yl)acetic acid

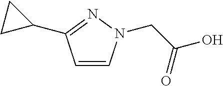 [SMILES: O=C(O)Cn1ccc(C2CC2)n1]

Prepared according to the method described for Preparation 126 using ethyl (3-cyclopropyl-1H-pyrazol-1-yl)acetate (Preparation 127). After acidifying to pH=4 with cHCl, EtOAc followed by water was added. The organic layer was collected and concentrated in vacuo to afford the title compound as a white solid (83%, 4.06 g).

LCMS Rt=1.16 minutes MS m/z 167 [M+H]⁺

### Preparation 129

### Ethyl (5-chloropyridin-2-yl)acetate

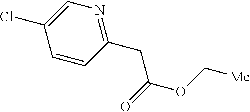 [SMILES: CCOC(=O)Cc1ccc(Cl)cn1]

Cesium carbonate (71 g, 218 mmol) was added to 2-bromo-5-chloropyridine (14 g, 73 mmol) and diethyl malonate (22 mL, 145 mmol) in dry 1,4-dioxane (280 mL) and the solution was degassed with argon for 30 minutes. Then copper (I) oxide (2.8 g, 14.55 mmol) and picolinic acid (3.6 g, 29 mmol) were added and the mixture was stirred in a sealed vessel at 130° C. for 24 hours. The mixture was cooled to room temperature, quenched with water (100 mL) and extracted with EtOAc (3×100 mL). The organic extracts were washed with water (200 mL), brine (200 mL), dried over sodium sulfate and evaporated in vacuo. The residue was purified by silica gel column chromatography eluting with EtOAc:Hexane 92:8 to afford the title compound as a yellow oil (54%, 8.0 M.

¹H NMR (400 MHz, DMSO-d₆): δ ppm 1.17 (t, 3H), 3.85 (s, 2H), 4.08 (q, 2H), 7.42 (d, 1H), 7.90 (dd, 1H), 8.54 (d, 1H).

### Preparation 130

### (5-Chloropyridin-2-yl)acetic acid

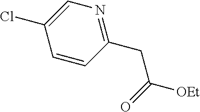 [SMILES: CCOC(=O)Cc1ccc(Cl)cn1]

The title compound was prepared according to the method described for Preparation 126 using ethyl (5-chloropyridin-2-yl)acetate (Preparation 129) to afford the title compound as a brown solid (51%, 3.5 g).

LCMS Rt=1.00 minutes MS m/z 172 [M+H]⁺

### Preparation 131

### Ethyl (5-fluoropyridin-2-yl)acetate

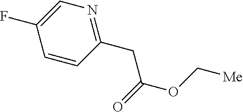 [SMILES: CCOC(=O)Cc1ccc(F)cn1]

The title compound was prepared according to the method described for Preparation 129 using 2-bromo-5-fluoropyridine to afford the title compound as a yellow oil (20%, 5 M.

¹H NMR (400 MHz, DMSO-d₆): δ ppm 1.17 (t, 3H), 3.84 (s, 2H), 4.08 (q, 2H), 7.42-7.45 (m, 1H), 7.67-7.72 (m, 1H), 8.48 (d, 1H).

### Preparation 132

### (5-Fluoropyridin-2-yl)acetic acid

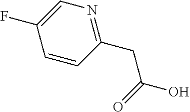 [SMILES: O=C(O)Cc1ccc(F)cn1]

The title compound was prepared according to the method described for Preparation 126 using ethyl (5-fluoropyridin-2-yl)acetate (Preparation 131) to afford the title compound as a brown solid (57%, 2.4 g).

¹H NMR (400 MHz, DMSO-d₆): δ ppm 3.75 (s, 2H), 7.41-7.44 (m, 1H), 7.65-7.70 (m, 1H), 8.47 (d, 1H), 12.50 (br s, 1H).

### Preparation 133

### (5-Bromopyridin-2-yl)acetic acid

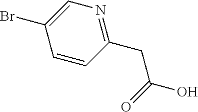 [SMILES: O=C(O)Cc1ccc(Br)cn1]

To a solution of diethyl(5-bromopyridin-2-yl)malonate (Preparation 134, 5.28 g, 16.70 mmol) in THF (50 mL) was added a solution of LiOH (2.10 g, 50.13 mmol) in water (12.5 mL) and the reaction was heated to 60° C. for 3 hours. The reaction was cooled and acidified to pH 3-4 with 2N HCl and diluted with 20% IPA in DCM. The organic layer was collected, dried over sodium sulphate and concentrated in vacuo. The crude residue was triturated with hexane to afford the title compound.

¹H NMR (400 MHz, DMSO-d₆): δ ppm 3.73 (s, 2H), 7.36 (d, 1H), 8.01 (m, 1H), 8.61 (d, 1H), 12.5 (s, 1H).

### Preparation 134

### Diethyl(5-bromopyridin-2-yl)malonate

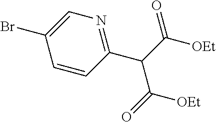 [SMILES: CCOC(=O)C(C(=O)OCC)c1ccc(Br)cn1]

To a suspension of 2-iodo-5-bromopyridine (2.0 g, 7.06 mmol), diethylmalonate (2.12 mL, 14.12 mmol) and cesium carbonate (6.88 g, 21.18 mmol) in dioxane (20 mL) was added copper iodide (268 mg, 1.41 mmol) followed by picolinic acid (346 mg, 2.82 mmol) and the reaction was heated to 80° C. for 16 hours. The reaction was cooled, filtered and the filtrate was concentrated in vacuo. The residue was diluted with EtOAc, washed with water, brine, dried over sodium sulphate and concentrated in vacuo. The residue was purified using silica gel column chromatography eluting with 2% EtOAc in hexanes to afford the title compound that was used directly in the next reaction.

### Preparation 135

### [4-Cyano-3-(trifluoromethyl)phenyl]acetic acid

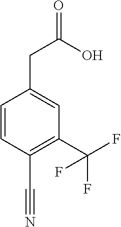 [SMILES: N#Cc1ccc(CC(=O)O)cc1C(F)(F)F]

Lithium diisopropylamide (13.8 mL, 24.8 mmol, 1.8M in THF) was added to 4-methyl-2-(trifluoromethyl)benzonitrile (2.30 g, 12.4 mmol) in THF (20 mL) at −78° C. and stirred for 5 minutes at −78° C. Excess solid carbon dioxide was added then the mixture was stirred at room temperature for 17 hours. Saturated aqueous ammonium chloride (10.5 mL) and EtOAc (20 mL) was added and the aqueous layer was acidified with 1M HCl. The mixture was extracted with EtOAc (3×15 mL) and the combined organic phases were dried over sodium sulphate and evaporated in vacuo to afford the title compound as a brown oil (88%, 2.52 g).

¹H NMR (400 MHz, CDCl₃): δ ppm 3.81 (s, 2H), 7.62 (d, 1H), 7.73 (s, 1H), 7.83 (d, 1H).

### Preparation 136

### [4-(methoxymethyl)-1H-1,2,3-triazol-1-yl]acetic acid

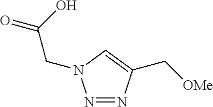 [SMILES: COCc1cn(CC(=O)O)nn1]

The title compound may be prepared according to the methods described for Preparation 117 using methylpropargylether.

### Preparation 137

### (3-isopropyl-1H-pyrazol-1-yl)acetic acid

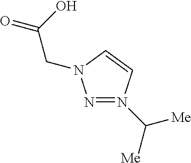

The title compound may be prepared according to the methods described for Preparation 127, isolating the higher running peak as the desired isomer, followed by Preparation and 126 using 3-isopropyl-1H-pyrazole and ethyl bromoacetate.

### Preparation 138

### (1E)-1-(dimethylamino)-2,4-dimethylpent-1-en-3-one

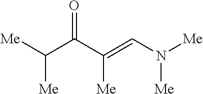 [SMILES: C/C(=C\N(C)C)C(=O)C(C)C]

2-Methyl-3-pentanone (5 g, 49.9 mmol) and DMF-DMA (10 mL, 74.9 mmol) were heated together in a sealed tube for 4 days. The reaction was concentrated in vacuo to afford an orange oil that was used directly in the next reaction (1.64 g, 21%).

### Preparation 139

### 3-isopropyl-5-methyl-1H-pyrazole

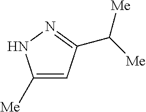

(1E)-1-(dimethylamino)-2,4-dimethylpent-1-en-3-one (Preparation 138, 1.64 g, 10.56 mmol) was heated with hydrazine hydrate (5 mL) at 100° C. for 3 hours. The reaction was allowed to cool and partitioned between EtOAc and water. The organic layer was collected, washed with brine, dried over MgSO₄ and concentrated in vacuo to afford an orange oil that was used directly in the next reaction (1.07 g, 82%).

### Preparation 140

### Ethyl (3-isopropyl-5-methyl-1H-pyrazol-1-yl)acetate and ethyl (5-isopropyl-3-methyl-1H-pyrazol-1-yl)acetate

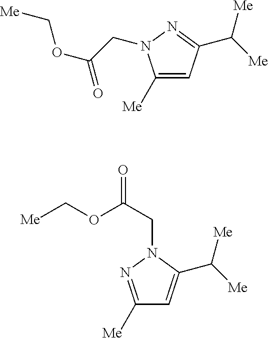 [SMILES: CCOC(=O)Cn1nc(C(C)C)cc1C ; CCOC(=O)Cn1nc(C)cc1C(C)C]

A suspension of 3-isopropyl-5-methyl-1H-pyrazole (Preparation 139, 1.07 g, 8.6 mmol), ethylbromoacetate (1 mL, 9.03 mmol) and potassium carbonate (3.57 g, 25.58 mmol) in DMF (10 mL) was stirred at room temperature for 18 hours. The reaction was diluted with EtOAc and washed with 1N HCl. The organic layer was collected, washed with brine, dried over MgSO₄ and concentrated in vacuo. The residue was purified using silica gel column chromatography eluting with 4:1 Hexane:EtOAc to obtain two regioisomers:

Major higher running peak: ethyl (3-isopropyl-5-methyl-1H-pyrazol-1-yl)acetate (607 mg, 34%).

Minor lower running peak: ethyl (5-isopropyl-3-methyl-1H-pyrazol-1-yl)acetate (102 mg, 6%).

### Preparation 141

### (3-isopropyl-5-methyl-1H-pyrazol-1-yl)acetic acid

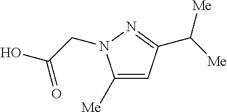 [SMILES: Cc1cc(C(C)C)nn1CC(=O)O]

A mixture of ethyl (3-isopropyl-5-methyl-1H-pyrazol-1-yl)acetate (Preparation 140, 571 mg, 2.72 mmol) and LiOH (342 mg, 8.15 mmol) in IMS (5 mL) and water (4 mL) was stirred at room temperature for 30 minutes. The reaction was acidified with 2M HCl and extracted with EtOAc. The organic layer was collected, washed with brine, dried over MgSO₄ and concentrated in vacuo to afford the title compound as a cream solid (328 mg, 66%).

### Preparation 142

### 4,6-Dichloro-5-(2,2-diethoxyethyl)pyrimidine

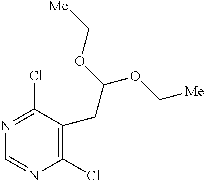 [SMILES: CCOC(Cc1c(Cl)ncnc1Cl)OCC]

Ammonium chloride (800 mg, 15.8 mmol) was added to (4,6-dichloropyrimidin-5-yl)acetaldehyde (2.00 g, 10.40 mmol) in ethanol (40 mL). The mixture was stirred at reflux for 20 hours. The reaction mixture was evaporated in vacuo. Water (20 mL) was added then extracted with ethyl acetate (30 mL). The organic phase was dried over sodium sulphate and evaporated in vacuo to afford the title compound as a colourless oil in 88% yield, 2.45 g.

¹H NMR (300 MHz, CDCl₃): δ ppm 1.1-1.3 (t, 6H), 3.2 (m, 2H), 3.4 (m, 2H), 3.7 (m, 2H), 4.8 (t, 1H), 8.6 (s, 1H).

### Biological Activity

Isolated TRK Enzyme assays use the HTRF KinEASE-TK kit (Cisbio Cat#62TK0PEJ) with recombinant His-tagged cytoplasmic domains of each TRK receptor sourced from Invitrogen (see table below). This activity-assay measures the phosphorylation of tyrosine residues within a substrate from the HTRF kit which has been validated by Cisbio for a variety of tyrosine kinases including the TRK receptors.

### Assay Details:

| | Invitrogen | Amino | FAC | FAC | Assay Reaction |
|---|---|---|---|---|---|
| Target | Cat# | acids | enzyme | ATP | Time |
| TRKA | PV3144 | aa 441-796 | 4 nM | 40 uM | 35 min |
| | (NTRK1) | | | | |
| TRKB | PV3616 | aa 526-838 | 1 nM | 1.4 uM | 40 min |
| | (NTRK2) | | | | |
| TRKC | PV3617 | aa 510-825 | 10 nM | 15 uM | 30 min |
| | (NTRK3) | | | | |

0.5 mM stock solutions of test compounds are prepared and serially diluted in 100% DMSO. A standard curve using the compound of Example 135 disclosed in WO2005/116035 of 150 uM is also prepared on each test plate. High percentage effect (HPE) is defined by 150 uM (using the compound of Example 135 as disclosed in WO2005/116035) and 0% effect (ZPE) is defined by 100% DMSO. Greiner low volume black plates containing 0.2 ul of serially diluted compound, standard and HPE/ZPE are created using the Bravo nanolitre dispenser.

1X enzyme buffer is prepared from 5X Enzymatic Buffer from the Cisbio KinEASE TK kit using MilliQ water. The buffer is then supplemented with 10 mM MgCl and 2 mM DTT (both from Sigma). In the case of TRKB, the buffer is also supplemented with 125 nM Supplement Enzymatic Buffer (SEB) from the Cisbio kit.

2X FAC of enzyme and 2X FAC ATP diluted in 1X complete enzyme buffer is incubated at room temperature for 20 minutes to preactivate the enzyme. Following this preactivation step, 5 ul/well of enzyme+ATP mix is added using a Multidrop Micro to the assay plate, spotted with 0.2 ul 100% DMSO compound. This is left for 20 mins at room temperature before adding 5 ul of 2 uM TK-substrate-Biotin (from the Cisbio kit) diluted in 1X enzyme buffer (1 uM FAC) using the Multidrop Micro. The reaction is incubated at room temperature for the optimized assay reaction time (see table). The reaction is stopped by adding 10 ul/well HTRF Detection Buffer containing 0.25 uM Streptavidin-XL665 (0.125 uM FAC) and 1:200 TK Antibody-Cryptate using a Multidrop.

After the Detection Reagent addition, plates are covered and incubated at room temperature for 60 minutes. HTRF signal is read using an Envision reader, measured as a ratio of emissions at two different wavelengths, 620 nm and 665 nm. Any compound that inhibits the action of the TRK kinase will have a lower fluorescence ratio value 665/620 nM than compounds which do not inhibit the TRK kinase. Test compound data are expressed as percentage inhibition defined by HPE and ZPE values for each plate. Percentage inhibition in the presence of test compound is plotted against compound concentration on a log scale to determine an IC₅₀ from the resultant sigmoid curve.

Cell Based Assays were carried out using Cell lines from DiscoveRx utilising their PathHunter technology and reagents in an antagonist assay:

| Target | DiscoveRx cell line Cat# | Cognate Neurotrophin |
|---|---|---|
| TRKA | 93-0462C3 | NGF |
| TRKA co expressed | 93-0529C3 | NGF |
| with p75 | | |
| TRKB | 93-0463C3 | BDNF |
| TRKB co expressed | 93-0530C3 | BDNF |
| with p75 | | |
| TRKC | 93-0464C3 | NT3 |
| TRKC co expressed | 93-0531C3 | NT3 |
| with p75 | | |

The assays are based upon DiscoveRx's proprietary Enzyme Fragment Complementation (EFC) technology. In the case of the TRK cell lines, the enzyme acceptor (EA) protein is fused to a SH2 protein and the TRK receptor of interest has been tagged with a Prolink tag.

Upon neurotrophin binding, the TRK receptor becomes phosphorylated, and the tagged SH2 protein binds. This results in functional complementation and restored β-Galactosidase activity which is can be measured using the luminescent Galacton Star substrate within the PathHunter reagent kits.

Generally, small molecule inhibitors bind to the kinase domain so are not competing with the neurotrophin (agonist) which binds to an extracellular site. This means that the IC₅₀ is a good measure of affinity and should be unaffected by concentration neurotrophin stimulant.

Cryopreserved PathHunter cells are used from either in-house produced batches or bulk batches bought directly from DiscoveRx. Cryopreserved cells are resuscitated, spun 1000 rpm for 4 min to remove freezing media, and resuspended in MEM+0.5% horse serum (both Invitrogen) to 5e⁵ cells/ml. The cells are then plated using a Multidrop into Greiner white tissue culture treated plates at 20 ul/well and incubated for 24 h at 37° C., 5% CO₂, high humidity. On the day of the assay, the cell plates are allowed to cool to room temperature for 30 min prior to the assay.

4 mM stock solutions of test compounds are prepared and serially diluted in 100% DMSO. A standard curve using the compound of Example 135, WO2005/116035 at a top concentration of 150 uM is also prepared on each test plate. High percentage effect (HPE) is defined by 150 uM of the compound of Example 135, WO2005/116035 and 0% effect (ZPE) is defined by 100% DMSO. Plates containing 1 ul of serially diluted compound, standard and HPE/ZPE are diluted 1/66 in assay buffer (PBS minus Ca²⁺, minus Mg²⁺ with 0.05% pluronic F127) using a Wellmate. Using a Platemate Plus, 5 ul of 1/66 diluted test compounds is then transferred to the cell plate and allowed to reach equilibrium by incubating for 30 min at room temperature before addition of agonist stimulus: 10 ul/well of 2 nM (0.571 nM FAC) of the cognate neurotrophin (Peprotech) diluted in agonist buffer (HBSS with 0.25% BSA). Final assay concentration of the test compounds is 8.66 μM, (the compound of Example 135, WO2005/116035 FAC is 0.325 uM). The plates are left at room temperature for a further 2 hours before addition of 10 ul of the DiscoveRx PathHunter detection reagent (made up by adding 1 part Galacton Star, 5 parts Emerald II and 19 parts Cell Assay Buffer as per the manufacturer's instructions).

After reagent addition, plates are covered and incubated at room temperature for 60 minutes. Luminescence signal is read using an Envision. Test compound data are expressed as percentage inhibition defined by HPE and ZPE values for each plate. Percentage inhibition in the presence of test compound is plotted against compound concentration on a log scale to determine an IC₅₀ from the resultant sigmoid curve.

### Brain Penetration Assays

In Vitro

MDCK-BCRP: MDCK-BCRP data can be collected according to the method described in “A 96-Well Efflux Assay To Identify ABCG2 Substrates Using a Stably Transfected MDCK II Cell Line” http://pubs.acs.org/doi/full/10.1021/mp050088t

Yongling Xiao, Ralph Davidson, Arthur Smith, Dennis Pereira, Sabrina Zhao, John Soglia, David Gebhard, Sonia de Morais, and David B. Duignan, Mol. Pharm., 2006, 3 (1), pp 45-54.

MDCK-MDR1: MDCK-MDR1 data can be collected according to the method described in “Are MDCK Cells Transfected with the Human MDR1 Gene a Good Model of the Human Intestinal Mucosa?” http://www.springerlink.com/content/qfhqlqbr4fnp3khf/fulltext.pdf

Fuxing Tang, Kazutoshi Horie, and Ronald T. Borchardt, Pharmaceutical Research, Vol. 19, No. 6, June 2002.

In Vivo

Brain penetration can be measured according to the method described in “Assessing brain free fraction in early drug discovery”. Read, K; Braggio, S., Expert Opinion Drug Metab Toxicol. (2010) 6 (3) 337-344.

Below are TrkA IC₅₀ data generated using the PV3144 TrkA enzyme assay. Where more than one reading was taken, the arithmetic mean is presented.

| | Trka enzyme | |
|---|---|---|
| Example | (IC50) | |
| 1 | 22.8 | nM |
| 2 | 84 | nM |
| 3 | 183 | nM |
| 4 | 74.3 | nM |
| 5 | 75.6 | nM |
| 6 | 21.3 | nM |
| 7 | 5.93 | nM |
| 8 | 32 | nM |
| 9 | 11.1 | nM |
| 10 | 13.2 | nM |
| 11 | 11.5 | nM |
| 12 | 60.7 | nM |
| 13 | 230 | nM |
| 14 | 131 | nM |
| 15 | 94.8 | nM |
| 16 | 147 | nM |
| 17 | 72.2 | nM |
| 18 | 202 | nM |
| 19 | 97.6 | nM |
| 20 | 120 | nM |
| 21 | 95.8 | nM |
| 22 | 59.5 | nM |
| 23 | 79.1 | nM |
| 24 | 59.8 | nM |
| 25 | 363 | nM |
| 26 | 24.5 | nM |
| 27 | 139 | nM |
| 28 | 111 | nM |
| 29 | 74.2 | nM |
| 30 | 89.7 | nM |
| 31 | 78.1 | nM |
| 32 | 101 | nM |
| 33 | 2560 | nM |
| 34 | 332 | nM |
| 35 | 10.9 | nM |
| 36 | 51.9 | nM |
| 37 | 21 | nM |
| 38 | 9.61 | nM |
| 39 | 5.27 | nM |
| 40 | 8.38 | nM |
| 41 | 10.7 | nM |
| 42 | 11.5 | nM |
| 43 | 15.7 | nM |
| 44 | 10.2 | nM |
| 45 | 36.1 | nM |
| 46 | 4.69 | nM |
| 47 | 5.71 | nM |
| 48 | 7.29 | nM |
| 49 | 9.7 | nM |
| 50 | 9.29 | nM |
| 51 | 78.7 | nM |
| 52 | 73.1 | nM |
| 53 | 216 | nM |
| 54 | 46.2 | nM |
| 55 | 12.9 | nM |
| 56 | 6.56 | nM |
| 57 | 66.1 | nM |
| 58 | 11.6 | nM |
| 59 | 12.3 | nM |
| 60 | 8.3 | nM |
| 61 | 25.4 | nM |
| 62 | 34.3 | nM |
| 63 | 136 | nM |
| 65 | 23.4 | nM |
| 66 | 4.45 | nM |
| 67 | 28.9 | nM |
| 68 | 51 | nM |
| 69 | 5.54 | nM |
| 70 | 144 | nM |
| 71 | 49.2 | nM |
| 72 | 117 | nM |
| 73 | 58.2 | nM |
| 74 | 20.9 | nM |
| 75 | 306 | nM |
| 76 | 315 | nM |
| 77 | 226 | nM |
| 78 | 118 | nM |
| 79 | 131 | nM |
| 80 | 150 | nM |
| 81 | 268 | nM |
| 82 | 76.1 | nM |
| 83 | 10.2 | nM |
| 84 | 27.1 | nM |
| 85 | 54.1 | nM |
| 86 | 35 | nM |
| 87 | 6.43 | nM |
| 88 | 75.9 | nM |
| 89 | 76.3 | nM |
| 90 | 18.5 | nM |
| 91 | 44.5 | nM |
| 92 | 747 | nM |
| 93 | 164 | nM |
| 94 | 922 | nM |
| 95 | 146 | nM |
| 96 | 139 | nM |
| 97 | 876 | nM |
| 98 | 1500 | nM |
| 99 | 430 | nM |
| 100 | 2190 | nM |
| 101 | 247 | nM |
| 102 | 96.6 | nM |
| 103 | 13.6 | nM |
| 104 | 91.1 | nM |
| 105 | 72.7 | nM |
| 106 | 663 | nM |
| 107 | 467 | nM |
| 108 | 369 | nM |
| 109 | 53.6 | nM |
| 111 | 31.8 | nM |
| 112 | 682 | nM |
| 113 | 58.9 | nM |
| 114 | 536 | nM |
| 115 | 126 | nM |
| 116 | 59.7 | nM |
| 117 | 1550 | nM |
| 118 | 8.82 | nM |
| 119 | 71.1 | nM |
| 120 | 18.6 | nM |
| 121 | 328 | nM |
| 122 | 4.24 | nM |
| 123 | 42.1 | nM |
| 124 | 15.1 | nM |
| 125 | 250 | nM |
| 127 | 1110 | nM |
| 128 | 1230 | nM |
| 129 | 201 | nM |
| 130 | 1750 | nM |
| 131 | 1110 | nM |
| 132 | 48.2 | nM |
| 133 | 9380 | nM |
| 134 | 583 | nM |
| 135 | 21.5 | nM |
| 136 | 25.8 | nM |
| 137 | 188 | nM |
| 138 | 31.1 | nM |
| 139 | 760 | nM |
| 140 | 223 | nM |
| 141 | 16.4 | nM |
| 142 | 29.5 | nM |
| 143 | 268 | nM |
| 144 | 111 | nM |
| 145 | 484 | nM |
| 146 | 246 | nM |
| 147 | 16.2 | nM |
| 148 | 185 | nM |
| 149 | 171 | nM |
| 150 | 85.3 | nM |
| 151 | 21.2 | nM |
| 152 | 71.2 | nM |
| 153 | 42 | nM |
| 154 | 12.4 | nM |
| 155 | 14.3 | nM |
| 156 | 1060 | nM |
| 157 | 15.6 | nM |
| 158 | 1350 | nM |
| 159 | 53.6 | nM |
| 160 | 86.3 | nM |
| 161 | 60.8 | nM |
| 162 | 9800 | nM |
| 163 | 3620 | nM |
| 164 | 31.8 | nM |
| 165 | 1040 | nM |
| 166 | 52.9 | nM |
| 167 | 72.9 | nM |
| 168 | 155 | nM |
| 169 | 459 | nM |
| 170 | 99.8 | nM |
| 171 | 255 | nM |
| 172 | 1080 | nM |
| 173 | 25.4 | nM |
| 174 | 27.3 | nM |
| 175 | 407 | nM |
| 176 | 107 | nM |
| 177 | 617 | nM |

All publications cited in this application are each herein incorporated by reference in their entirety.

Although the invention has been described above with reference to the disclosed embodiments, those skilled in the art will readily appreciate that the specific experiments detailed are only illustrative of the invention. It should be understood that various modifications can be made without departing from the spirit of the invention.

Accordingly, the invention is limited only by the following claims.

1. A compound of Formula I: 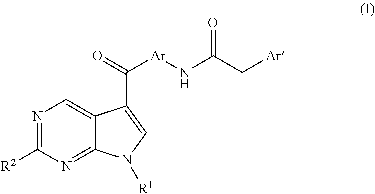
or a pharmaceutically acceptable salt thereof, wherein
R¹ is C₂₋₄ alkyl optionally substituted by 1 or 2 OH, optionally wherein a methylene group is replaced by an oxetane group,
or R¹ is C₃₋₆ cycloalkyl optionally substituted by OH,
or R¹ is (C₃₋₆ cycloalkyl)C₁₋₃ alkyl optionally substituted by 1 or 2 OH;
R² is H, OH or NH₂;
Ar is a ring system selected from 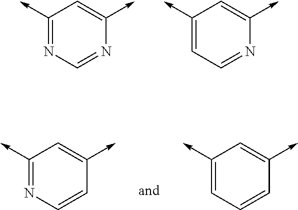
which ring system is optionally substituted on a carbon atom by C₁₋₃ alkyl, CN or C₁₋₃ alkoxy;
and Ar′ is a ring system selected from 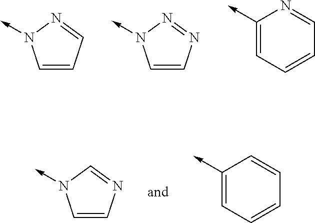
which ring system is optionally substituted on a carbon atom by 1 or 2 substituents independently selected from:
halo, ═O, CN, C₁₋₃ alkyl optionally substituted by one or more F or C₁₋₃ alkoxy, C₁₋₃ alkoxy optionally substituted by one or more F, C₃₋₆ cycloalkyl, C₃₋₆ cycloalkyloxy and SO₂(C₁₋₃ alkyl).

2. The compound according to claim 1 or a pharmaceutically acceptable salt thereof, wherein R¹ is selected from:

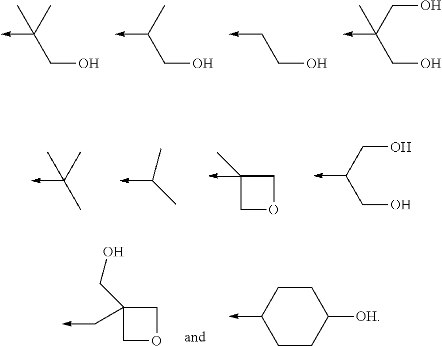

3. The compound according to claim 1 or a pharmaceutically acceptable salt thereof wherein Ar′ is a ring system selected from: 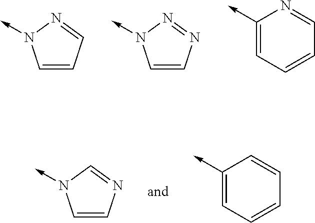
which ring system is substituted on a carbon atom by 1 or 2 substituents independently selected from:
F, Cl, ═O, CN, CH₃, CF₃, OCF₃, cyclopropyl, cyclopropyloxy and SO₂CH₃.

4. The compound according to claim 1 or a pharmaceutically acceptable salt thereof, wherein R₁ is selected from: 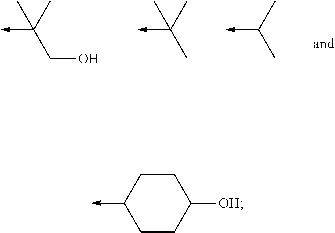
R₂ is H or NH₂;
Ar is selected from 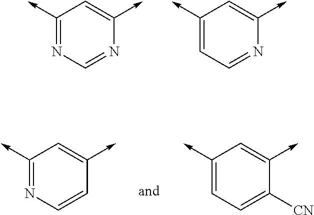
and Ar′ is a ring system selected from

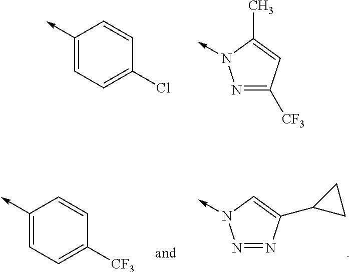

5. A compound selected from:
2-(4-chlorophenyl)-N-(4-{[7-(trans-4-hydroxycyclohexyl)-7H-pyrrolo[2,3-d]pyrimidin-5-yl]carbonyl}pyridin-2-yl)acetamide;
N-{5-[2-Amino-7-(2-hydroxy-1,1-dimethyl-ethyl)-7H-pyrrolo[2,3-d]pyrimidine-5-carbonyl]-2-cyano-phenyl}-2-(4-trifluoromethyl-phenyl)-acetamide;
N-[2-(7-tert-Butyl-7H-pyrrolo[2,3-d]pyrimidine-5-carbonyl)-pyridin-4-yl]-2-(4-cyclopropyl-[1,2,3]triazol-1-yl)-acetamide;
N-[4-(2-Amino-7-isopropyl-7H-pyrrolo[2,3-d]pyrimidine-5-carbonyl)-pyridin-2-yl]-2-(4-chloro-phenyl)-acetamide;
2-(4-Chloro-phenyl)-N-[2-(7-isopropyl-7H-pyrrolo[2,3-d]pyrimidine-5-carbonyl)-pyridin-4-yl]-acetamide;
N-{2-[2-Amino-7-(2-hydroxy-1,1-dimethyl-ethyl)-7H-pyrrolo[2,3-d]pyrimidine-5-carbonyl]-pyridin-4-yl}-2-(4-chloro-phenyl)-acetamide;
2-(4-chlorophenyl)-N-(6-{[7-(propan-2-yl)-7H-pyrrolo[2,3-d]pyrimidin-5-yl]carbonyl}pyrimidin-4-yl)acetamide; and
2-[5-methyl-3-(trifluoromethyl)-1H-pyrazol-1-yl]-N-(4-{[7-(propan-2-yl)-7H-pyrrolo[2,3-d]pyrimidin-5-yl]carbonyl}pyridin-2-yl)acetamide;
or a pharmaceutically acceptable salt thereof.

6. A pharmaceutical composition comprising a compound of the formula (I) or a pharmaceutically acceptable salt thereof, as defined in claim 1, and a pharmaceutically acceptable carrier.

7-11. (canceled)

12. A method of treatment of a mammal, to treat a disease for which an Trk receptor antagonist is indicated, comprising treating said mammal with an effective amount of a compound of the formula (I) or a pharmaceutically acceptable salt thereof, as defined in claim 1.

13. A method of treatment of pain or cancer in a mammal, comprising treating said mammal with an effective amount of a compound of the formula (I) or a pharmaceutically acceptable salt thereof, as defined in claim 1.

14. A method of treatment of a mammal, to treat a disease for which a TrK receptor antagonist is indicated, comprising administering to said mammal a therapeutically effective amount of a compound or salt according to claim 1 in combination with a further drug substance.
